(19) United States
(12) Patent Application Publication
(10) Pub. No.: US 2003/0166601 A1
(43) Pub. Date: Sep. 4, 2003

(54) Novel colloid synthetic vectors for gene therapy

(76) Inventors: Martin C. Woodle, Bethesda, MD (US); Cheng Cheng, Rockville, MD (US); Puthupparampil Scaria, Montgomery Village, MD (US); Kas Subramanian, Edison, NJ (US); Richard Titmas, Boxford, MA (US); Jingping Yang, N. Potomac, MD (US); Joerg Frei, Helstein (CH); Helmut Mett, Neuenburg (DE); Jaroslav Stanek, Arlesheim (CH)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/290,406

(22) Filed: Nov. 6, 2002

Related U.S. Application Data
(63) Continuation of application No. 09/475,305, filed on Dec. 30, 1999, now abandoned.

(51) Int. Cl. A61K 48/00; C12N 15/00

(52) U.S. Cl. 514/44; 435/320.1

(57) ABSTRACT

Non-naturally occurring vector for gene therapy are provided, comprised of chemically defined reagents, where the vector is self-assembling and where the vector comprises (1) a core complex comprising a nucleic acid and (2) at least one complex forming reagent, where the vector has fusogenic activity. The vector optionally may contain reagents permitting fusion with cell membranes and nuclear uptake. The vector also may contain an outer shell moiety that is anchored to the core complex, whereby the outer shell stabilizes the complex, protects it from unwanted interactions and enhances delivery of the nucleic acid into a target tissue or cell. The outer shell optionally may be sheddable, that is, it may be designed such that it dissociates from the vector upon entry into the target cell or tissue.

This is a continuation of U.S. application Ser. No. 09/475,305, filed Dec. 30, 1999, incorporated herein by reference in its entirety.

### BACKGROUND OF THE INVENTION

1. Field of the Invention

This invention provides compositions and methods for ex vivo, local, and systemic nucleic acid delivery.

2. Description of the Related Art

A critical requirement for the success of gene therapy is the ability to deliver the therapeutic nucleic acid of interest to the target tissue and cell types without substantial distribution to non-target tissues. A variety of synthetic molecules have been tested for their ability to deliver nucleic acids into cells, i.e. synthetic vectors. Conventional approaches to delivery of synthetic vectors has tended to concentrate on use of cationic lipid or cationic polymer-based systems. See, for example, Barron, et al., Hum. Gene Ther. 9:315-323 (1998),; Gao et al., Gene Therapy 2:710 (1995); Zelphati et al., J. Controlled Release 41:99 (1996)) or cationic polymers (Boussif et al., Proc. Natl. Acad. Sci. U.S.A. 92:7297 (1995)); Goula et al., Gene Therapy 5:1291 (1995)); Chemin, et al., J Viral Hepat 5:369 (1995)); Kwoh et al., Biochim. Biophys. Acta 1444:171 (1999)); Wagner, J. Controlled Release 53:155 (1998)); and Plank et al., Hum. Gene. Ther. 10:319 (1999)).

Complexes of plasmid DNA encoding proteins with cationic lipids or cationic polymers (respectively referred to as “lipoplexes” and “polyplexes”) transfect cells to give most efficient protein expression usually when the net charge on the complex is positive (charge ratios (+/−) greater than 1). Similarly, antisense or ribozyme oligonucleotides, with a sequence specific for an mRNA encoding a protein, complexed with similar or identical reagents can be delivered to cells in culture to give most effective inhibition of the specified protein usually when the net charge on the complex is positive. Some other preparations for nucleic acids developed include conjugates of polycations such as polylysine with targeting ligands such as FGF2 protein, liposomes encapsulating the nucleic acid in the internal entrapped aqueous phase, enveloped virus fused to liposomes encapsulating the nucleci acid such as the so-called HVJ-liposome, and a variety of emulsion preparations where the nucleic acid is sequestered into the non-aqueous phase of an emulsion or microparticle. Despite a variety of preparations, in most cases, though, the nucleic acid is bound into a colloid complexes by a complexation or encapsulation method. Many efforts have been made to prepare compositions that can provide delivery vectors for plasmids, oligonucleotides, and other forms of nucleic acids for the purpose of attaining a desired pharmacological benefit but to date these preparations still lack in vivo stability, specificity for target tissues and cells, and the capacity to provide adequate level of nucleic acid activity in the target tissues and cells. The mechanism by which these colloidal complexes are internalized is not understood, but is thought to depend on net charge in the complex and it is assumed that the positive surface charge of the complex and the negative surface charge of the cells play a major role in cellular uptake of the complexes as well as many other interactions with biological systems.

A major disadvantage of lipoplexes and polyplexes is their tendency to interact nonspecifically with a wide variety of cells, contributing to several unwanted effects. In addition, the complexes can interact electrostatically with negatively charged proteins and other components in serum, leading to surface modification or destabilization of the complexes and other unfavorable effects or cellular interactions.

A further problem with conventional complexes is their lack of colloidal stability. This instability results in aggregation of the complexes into large particles, especially at or near neutral charge ratios, and causes difficulty with long term storage. A number of approaches have been tried to overcome this problem. For example, one of the simplest approaches is by surface modification with a steric polymer such as poly(ethyleneglycol) (PEG). (Scaria et al., 1999, Program of the American Society of Gene Therapy meeting held at Washington D.C. on June 9-13, p221 a, abs# 878, Meyer et al., 1998, J. Biol. Chem. 273,15621-15627; Choi et al., 1998, Bioconjug. Chem. 9,708-718; Choi et al., 1998, J. Controlled Release 54,39-48; Kwoh et al., 1999, Biochim. Biophys. Acta 1444,171-190; Vinogradov et al., 1998, Bioconjug Chem 9:805-12; Zelphati et al., 1998, Gene. Ther. 5, 1272-1282; Phillips, 1997, International Business Communications meeting held at Annapolis, Md. on Jun. 23-24, 1997; and Woodle et al., 1992, Biophys. J. 61, 902-10; E. Schacht et al., WO 9819710). A steric coating on the surface of the complex can enhance colloidal stability.

Such steric coatings also minimize interactions with target and non-target tissue and cells as well as serum components, an undesired effect in the case of target tissues and cells. Modification of lipoplexes and polyplexes with PEG (PEGylation), however, has a significant deleterious effect on the biological activity of the complex. In addition to the desirable effect of inhibiting non-specific and unwanted binding to the cell surface, use of a steric surface may adversely impact binding to target tissues and cells. Furthermore, it may adversely impact subsequent steps in the DNA delivery process once binding to target cells has occurred. For example, PEGylation leads to poor overall levels of expression of the protein encoded by the DNA component of the complex (Scaria and Philips supra).

Schacht et al. (WO 9819710) state that a particularly advantageous construction method involves stepwise construction first of nucleic acid complexes with cationic polymer molecules followed by a second step where the cationic polymer molecules are covaently coupled to a hydrophilic polymer block or to one or more targeting moieties and/or other bioactive molecules. A self-assembled hydrophilic polymer coating is constructed using A-B type linear block copolymers and such coatings can provide stabilization, though the complexes thus formed often still are destabilized quite quickly. Accordingly, Schacht describes a 2-stage procedure for assembly of the complexes where hydrophilic polymer and targeting moieties or other bioactive molecules are covalently attached to a preexisting colloid, i.e. particle. In addition, the covalent attachment of the hydrophilic polymer uses a polymer having multivalent covalent attachments so that cross-linking occurs in the surface coating of the complex. Such complexes have a number of limitations. Importantly, this kind of construction inevitably results in many different chemical structures which have significant differences in their activities including both desired and undesired ones. Furthermore, control of the amounts of each structure produced is difficult if not impossible. Importantly, the first hydrophilic polymer coupling events form a rudimentary steric coat that reduces the further occurance of coupling reactions so that the process becomes self-limiting. When greater amounts of surface bound polymer are needed than the self-limiting coupling permits then the resulting coat is inadequate. Furthermore, a complete control over the coupling reaction in terms of which chemical species are formed is very difficult, if not impossible, when the conjugates are formed on the surface of a preexisting particle. Yet further difficulties are a need to protect from unwanted reactions or conjugations to the nucleic acid component but which is not easily fulfilled. Still other difficulties with a complex prepared with a 2-step method is a requirement that the core complex be prepared at a positive surface charge.

Finally, when a complex successfully reaches a target tissues and cell, it must be able to bind efficiently with the target tissues and cell membrane and deliver efficiently the nucleic acid contents to the intracellular compartment where its activity can be exerted. Conventional complexes tend to perform these steps only poorly, leading to inefficient and/or inadequate levels of gene expression.

It is apparent, therefore, that gene delivery vectors having improved target specificity and in vivo stability and which are relatively homogenous while being comprised of chemically defined species are greatly to be desired. In particular, it is desirable that the stable gene delivery vectors have an improved outer steric layer that provides enhanced target specificity, in vivo and colloidal stability, and enhanced target specificity. Furthermore, it is desirable that the vectors demonstrate improved cell entry and intracellular trafficking permiting enhanced nucleic acid therapeutic activity such as gene expression.

### SUMMARY OF THE INVENTION

It is therefore an object of the invention to provide a non-naturally occurring vector for gene therapy comprised of chemically defined reagents, where the vector is self-assembling and where the vector comprises (1) a core complex comprising a nucleic acid and (2) at least one complex forming reagent, where the vector has fusogenic activity. The vector optionally may contain reagents permitting fusion with cell membranes and nuclear uptake. The vector also may contain an outer shell moiety that is anchored to the core complex, whereby the outer shell stabilizes the complex, protects it from unwanted interactions and enhances delivery of the nucleic acid into a target tissue or cell. The outer shell optionally may be sheddable, that is, it may be designed such that it dissociates from the vector upon entry into the target cell or tissue.

It is a further object of the invention to provide methods of making these vectors, pharmaceutical compositions comprising the vectors, and methods of using the vectors and pharmaceutical compositions to treat patients.

In accordance with these objects there has been provided a non-naturally occurring gene therapy vector comprising an inner shell comprising (1) a core complex comprising a nucleic acid and at least one complex forming reagent where the vector has fusogenic activity. The vector may further comprise a fusogenic moiety. The fusogenic moiety may comprise a shell that is anchored to the core complex, or the fusogenic moiety may be incorporated directly in the core complex.

In another embodiment, the vector comprises an outer shell moiety that stabilizes the vector and reduces nonspecific binding to proteins and cells. The outer shell moiety may comprise a hydrophilic polymer.

In another embodiment, the vector comprises a fusogenic moiety. The outer shell moiety may be anchored to the fusogenic moiety, or may be anchored to the core complex.

In yet another embodiment, the vector may comprise a mixture of at least two outershell reagents. The outershell reagents may each comprise a hydrophilic polymer that reduces nonspecific binding to proteins and cells, and wherein the polymers have substantially different sizes.

In still another embodiment, the vector may contain a targeting moiety that enhances binding of the vector to a target tissue and cell population. The targeting moiety may be contained in the outer shell moiety.

In yet another embodiment, the complex-forming reagent is selected from the group consisting of a lipid, a polymer, and a spermine analogue complex. The complex-forming reagent may be a lipid selected from the group consisting of the lipids shown in FIGS. 2.1 and 2.2. In particular, the complex-forming lipid agent may be is selected from the group consisting of phosphatidylcholine (PC), phosphatidylethanolamine (PE), dioleoylphosphatidylethanolamine (DOPE), dioleoylphosphatidylcholine (DOPC), cholesterol and other sterols, N-1-(2,3-dioleyloxy)propyl-N,N,N-trimethylammonium chloride (DOTMA), 1,2-bis (oleoyloxy)-3-(trimethylammonia) propane (DOTAP), phosphatidic acid, phosphatidylglycerol, phosphatidylinositol, glycolipids comprising two optionally unsaturated hydrocarbon chains containing about 14-22 carbon atoms, sphingomyelin, sphingosine, ceramide, terpenes, cholesterol hemisuccinate, cholesterol sulfate, diacylglycerol, 1,2-dioleoyl-3-dimethylammonium propanediol (DODAP), dioctadecyldimethylammonium bromide (DODAB), dioctadecyldimethylammonium chloride (DODAC), dioctadecylamidoglycylspermine (DOGS), 1,3-dioleoyloxy-2-(6-carboxyspermyl)propylamide (DOSPER), 2,3-dioleyloxy-N-[2-(sperminecarboxamido)ethyl]-N,N-dimethyl-1-propanaminium trifluoroacetate (DOSPA or Lipofectamine 7), hexadecyltrimethyl-ammonium bromide (CTAB), dimethyl-dioctadecylammonium bromide (DDAB), 1,2-dimyristyloxypropyl-3-dimethyl-hydroxy ethyl ammonium bromide (DMRIE), dipalmitoylphosphatidylethanolamylspermine (DPPES), dioctylamineglycine-spermine (C8Gly-Sper), dihexadecylamine-spermine (C18-2-Sper), aminocholesterol-spermine (Sper-Chol), 1-[2-(9(Z)-octadecenoyloxy)ethyl]-2-(8(Z)-heptadecenyl)-3-(2-hydroxyethyl)imidazolinium chloride (DOTIM), dimyristoyl-3-trimethylammonium-propane (DMTAP), 1.2-dimyristoyl-sn-glycero-3-ethylphosphatidylcholine (EDMPC or DMEPC), lysylphosphatidylethanolamine (Lys-PE), cholestryl-4-aminoproprionate (AE-Chol), spermadine cholestryl carbamate (Genzyme-67), 2-(dipalmitoyl-1,2-propandiol)-4-methylimidazole (DPIm), 2-(dioleoyl-1,2-propandiol)-4-methylimidazole (DOIm), 2-(cholestryl-1-propylamine carbamate)imidazole (ChIm), N-(4-pyridyl)-dipalmitoyl-1,2-propandiol-3-amine (DPAPy), 3β-[N—(N′,N′-dimethylaminoethane)carbamoyl]cholesterol (DC-Chol), 3β-[N—(N′,N′,N′-trimethylaminoethane)carbamoyl] cholesterol (TC-CHOL-gamma-d3), 1,2-dioleoyl-sn-glycero-3-succinate, 1,2-dioleoyl-sn-glycero-3-succinyl-2-hydroxethyl disulfide ornithine conjugate (DOGSDSO), 1,2-dioleoyl-sn-glycero-3-succinyl-2-hydroxethyl hexyl orithine conjugate (DOGSHDO), N,N^{I},N^{II},N^{III}-tetramethyl-N,N^{I},N^{II},N^{III}-tetrapalmityolspermine (TM-TPS), 3-tetradecylamino-N-tert-butyl-N′-tetradecylpropionamidine (vectamidine or diC14-amidine), N-[3-[2-(1,3-dioleoyloxy)propoxy-carbonyl]propyl]-N,N,N-trimethylammonium iodide (YKS-220), and O,O′-Ditetradecanoyl-N-(alpha-trimethylammonioacetyl)diethan olamine chloride (DC-6-14).

The complex forming reagent also may be a compound of formula I

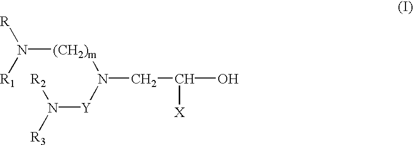

where m is 3 or 4;

Y signifies a group —(CH₂)ₙ—, in which n is 3 or 4, or may also signify a group —(CH₂)ₙ—, in which n is an integer from 5 to 16, or may also signify a group —CH₂—CH═CH—CH₂—, if R₂ is a group —(CH₂)₃—NR₄R₅ and m is 3;

R₂ is hydrogen or lower alkyl or may also signify a group —(CH₂)₃—NR₄R₅ if m is 3;

R₃ is hydrogen or alkyl or may also signify a group —CH₂—CH(—X′)—OH, if R₂ is a group —(CH₂)₃—NR₄R₅ and m is 3;

X and X′, independently of one another, signify hydrogen or alkyl;

the radicals R, R₁, R₄ and R₅, independently of one another, are hydrogen or lower alkyl; with the proviso that the radicals R, R₁, R₂, R₃ and X cannot all together signify hydrogen or methyl, if m is 3 and Y signifies a group —(CH₂)₃—; and their pharmaceutically acceptable salts.

In a further embodiment, the complex forming reagent comprises a mixture of at least two complex forming reagents.

In a still further embodiment, the complex forming reagent possesses one or more additional activities selected from the group consisting of cell binding, biological membrane fusion, endosome disruption, and nuclear targeting.

In other embodiments, the nucleic acid is selected from the group consisting of a recombinant plasmid, a replication-deficient plasmid, a mini-plasmid, a recombinant viral genome, a linear nucleic acid fragment, an antisense agent, a linear polynucleotide, a circular polynucleotide, a ribozyme, a cellular promoter, and a viral genome.

The core complex also may further comprises a nuclear targeting moiety that enhances nuclear binding and/or uptake. The nuclear targeting moiety may be selected from the group consisting of a nuclear localization signal peptide, a nuclear membrane transport peptide, and a steroid receptor binding moiety. The nuclear targeting moiety may be anchored to the nucleic acid in the core complex.

In still further embodiments, the fusogenic moiety comprises at least one moiety selected from the group consisting of a viral peptide, an amphiphilic peptide, a fusogenic polymer, a fusogenic polymer-lipid conjugate, a biodegradable fusogenic polymer, and a biodegradable fusogenic polymer-lipid conjugate. The fusogenic moiety mauy be a viral peptide selected from the group consisting of MLV env peptide, HA env peptide, a viral envelope protein ectodomain, a membrane-destabilizing peptide of a viral envelope protein membrane-proximal domain, a hydrophobic domain peptide segment of a viral fusion protein, and an amphiphilic-region containing peptide, wherein the amphiphilic-region containing peptide is selected from the group consisting of melittin, the magainins, fusion segments from H. influenza hemagglutinin (HA) protein, HIV segment I from the cytoplasmic tail of HIV1 gp41, and amphiphilic segments from viral env membrane proteins.

In yet further embodiments, wherein the complex forming reagent is a polymer having the structure:

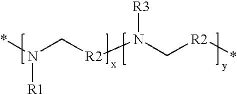

wherein R1 and R3 independently are a hydrocarbon or a hydrocarbon substituted with an amine, guanidinium, or imidazole moiety, wherein R1 and R3 can be identical or different; and

R2 is a lower alkyl group. The complex forming reagent also may be a polymer having the structure:

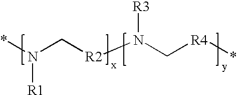

wherein R1 and R3 independently are a hydrocarbon or a hydrocarbon substituted with an amine, guanidinium, or imidazole moiety, wherein R1 and R3 can be identical or different; and

R2 and R4 independently are lower alkyl groups.

In other embodiments, the fusogenic moiety is a polymer having the structure:

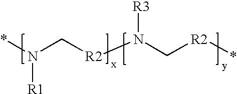

wherein R1 is a hydrocarbon or a hydrocarbon substututed with an amine, guanidinium, or imidazole moiety;

R2 is a lower alkyl group;

and R3 is a hydrocarbon or a hydrocarbon substututed with a carboxyl, hydroxyl, sulfate, or phosphate moiety. The fusogenic moiety also may be a polymer having the structure:

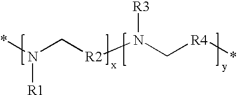

wherein R1 is a hydrocarbon or a hydrocarbon substututed with an amine, guanidinium, or imidazole moiety;

R2 and R4 independently are lower alkyl groups, and R3 is a hydrocarbon or a hydrocarbon substituted with a carboxyl, hydroxyl, sulfate, or phosphate moiety. The fusogenic moiety also may be a membrane surfactant polymer-lipid conjugate. The surfactant polymer-lipid conjugate may be selected from the group consisting of Thesit™, Brij 58™, Brij 78™, Tween 80™, Tween 20™, C₁₂E₈, C₁₄E₈, C₁₆E₈ (CₙEₙ=hydrocarbon poly(ethylene glycol) ether where C represents hydrocarbon of carbon length N and E represents poly(ethylene glycol) of degree of polymerization N), Chol-PEG 900, analogues containing polyoxazoline or other hydrophilic polymers substituted for the PEG, and analogues having fluorocarbons substituted for the hydrocarbon.

In still further embodiments, the inner shell is anchored to the outer shell moiety via a covalent linkage that is degradable by chemical reduction or sulfhydryl treatment. The inner shell may be anchored to the outer shell moiety via a covalent linkage that is degradable at a pH of 6.5 or below. The covalent linkage may be selected from the group consisting of

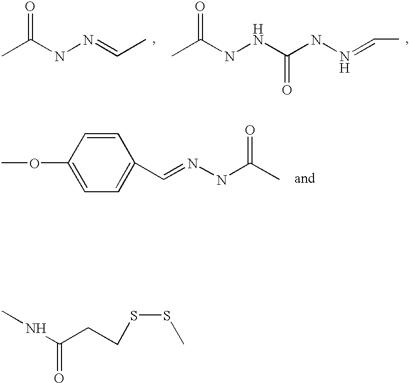

In other embodiments, the outer shell comprises a protective polymer conjugate where the polymer exhibits solubility in both polar and non-polar solvents. The polymer in the protective steric polymer conjugate may be selected from the group consisting of PEG, a polyacetal polymer, a polyoxazoline, a polyoxazoline polymer block with end-group conjugation, a hydrolyzed dextran polyacetal polymer, a polyoxazoline, a polyethylene glycol, a polyvinylpyrrolidone, polylactic acid, polyglycolic acid, polymethacrylamide, polyethyloxazoline, polymethyloxazoline, polydimethylacrylamide, polyvinylmethylether, polyhydroxypropyl methacrylate, polyhydroxypropylmethacrylamide, polyhydroxyethyl acrylate, polyhydroxyethyloxazoline, polyhydroxypropyloxazoline and polyaspartamide, and a polyvinyl alcohol.

In still further embodiments, the vector contains a targeting element selected from the group consisting of a receptor ligand, an antibody or antibody fragment, a targeting peptide, a targeting carbohydrate molecule or a lectin. The targeting element may be selected from the group consisting of vascular endothelial cell growth factor, FGF2, somatostatin and somatostatin analogs, transferrin, melanotropin, ApoE and ApoE peptides, von Willebrand's Factor and von Willebrand's Factor peptides; adenoviral fiber protein and adenoviral fiber protein peptides; PD1 and PD1 peptides, EGF and EGF peptides, RGD peptides, folate, pyridoxyl, and sialyl-Lewis^{x} and chemical analogues.

In accordance with another object of the invention, there has been provided compounds having the formula I

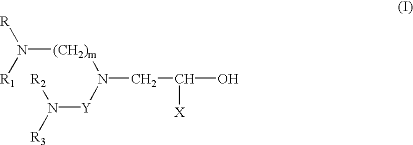

wherein m is 3 or 4; Y signifies a group —(CH₂)ₙ—, in which n is 3 or 4, or may also signify a group —(CH₂)ₙ—, in which n is an integer from 5 to 16, or may also signify a group —CH₂—CH═CH—CH₂—, if R₂ is a group —(CH₂)₃—NR₄R₅ and m is 3; R₂ is hydrogen or lower alkyl or may also signify a group —(CH₂)₃—NR₄R₅ if m is 3; R₃ is hydrogen or alkyl or may also signify a group —CH₂—CH(—X′)—OH, if R₂ is a group —(CH₂)₃—NR₄R₅ and m is 3; X and X′, independently of one another, signify hydrogen or alkyl; and the radicals R, R₁, R₄ and R₅, independently of one another, are hydrogen or lower alkyl; with the proviso that the radicals R, R₁, R₂, R₃ and X cannot all together signify hydrogen or methyl, if m is 3 and Y signifies a group —(CH₂)₃—; and their pharmaceutically acceptable salts.

In another aspect of the invention there has been provided a pharmaceutical composition comprising the vector described above, together with a pharmaceutically acceptable diluent or excipient.

In accordance with another aspect of the invention there has been provided a method for forming a self-assembling core complex of the type described above, where the method comprises the step of feeding a stream of a solution of a nucleic acid and a stream of a solution of a core complex-forming moiety into a static mixer, wherein the streams are split into inner and outer helical streams that intersect at several different points causing turbulence and thereby promoting mixing that results in a physicochemical assembly interaction.

In accordance with still another aspect of the invention, there has been provided methods of treating a disease in a patient, comprising administering to the patient a therapeutically effective amount of a vector as described above.

In accordance with yet another aspect of the invention there has been provided a non-naturally occurring gene therapy vector comprising an inner shell comprising: (1) a core complex comprising a nucleic acid and at least one complex forming reagent; (2) a nuclear targeting moiety; (3) a fusogenic moiety; and (4) an outer shell comprising (i) a hydrophilic polymer that stabilizes the vector and reduces nonspecific binding to proteins and cells and (ii) a tageting moiety that provides binding to target tissues and cells, where the outer shell is linked via a cleavable linkage that enables the outer shell to be shed.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 show a diagram of non-naturally occurring vectors comprising (1) a core complex comprising a nucleic acid and at least one complex forming reagent and optionally reagents providing fusion with cell membranes and nuclear uptake, and (2) an optional outer shell anchored to the core complex optionally with a cleavable segment, and (3) an optional exposed ligand anchored either to the core complex or the outer shell (structure E).

FIGS. 2.1-2.2 shows the chemical structures of cationic lipids.

FIGS. 3.1-3.5 shows diagrams of structures formed by substituted aminoethanols and nucleic acids.

FIG. 4 shows small particle size distribution and homogeneity of complexes formed by substituted aminoethanols and nucleic acids.

FIG. 5 shows luciferase expression resulting from transfection of in vivo tissues following intravenous administration to mice of core complexes formed from commercially obtained cationic lipids, formed from substituted aminoethanols, and from commercially obtained (ExGen) or synthesized (Lp500) linear PEI cationic polymers.

FIG. 6 shows GM-CSF expression resulting from transfection of in vivo tissues following intravenous administration to mice of core complexes formed from commercially obtained cationic lipids.

FIG. 7 shows luciferase expression resulting from transfection of in vivo tissues following intravenous administration to mice of core complexes formed from commercially obtained cationic lipids with a shell formed by inclusion of fusogenic surfactants (containing hydrophilic PEG polymer with a low molecular weight—less than 2000 daltons) or steric surfactants (containing hydrophilic PEG polymer with a high molecular weight—equal to or greater than 2000 daltons).

FIG. 8 shows increased expression by addition of a fusogenic peptide (K14-Fuso) derived from HA protein to polylysine core complexes.

FIG. 9 shows cleavage of hydrazone linkages at acidic pH.

FIG. 10A shows diagrams of some methods for incorporation of NLS into the payload nucleic acid and FIG. 10B shows increased expression by linear DNA with PNA linked NLS bound to it versus linear DNA alone.

FIG. 11 shows dependence of particle size distribution on charge ratio of PEI/DNA and PEI-PEG5000/DNA complexes. Error bars represent the standard deviation of the particle size distribution. DNA (Salmon sperm) concentration: 100 μg/ml; Mol % PEG in the complex: 5.0 FIG. 12 shows particle size stability of a PEI-PEG5000/DNA complex containing 100 μg/ml salmon sperm DNA; Charge ratio 1 (+/−), 5 Mol % PEG in the complex: 5.0. Error bars represent the standard deviation of the particle size distribution

FIG. 13 shows the effect of PEG on the aggregation of PEI/DNA complex in presence of serum. Particle size of PEI or PEI-PEG/DNA complexes containing varying mole % PEG before and after incubation with 10% serum. Samples incubated with serum at 37° C. for 30 min were dialyzed extensively against a dialysis bag with a 1,000,000 MW cut off, before measuring the particle size. Error bars are standard deviations of the distribution.

FIG. 14 shows a schematic representation of the effect of PEG of different molecular weight, on protein mediated aggregation of positively charged PEI/DNA complexes.

FIG. 15A shows prolonged blood clearance of I¹²⁵-DNA complexes with anchored PEG or Polyoxazoline polymers in mice and FIG. 15B shows reduced lung uptake of I¹²⁵-DNA complexes with anchored PEG or Polyoxazoline polymers in mice.

FIG. 16 shows the particle size of a PEI-ss-PEG500O/DNA complex. Bar 1 shows the average size of the particles made by complexing 250 μg/ml DNA(Salmon Sperm) with PEI-ss-PEG5000 (PEI-ss-PEG5000 containing 11 mol % PEG) at 1:1 charge ratio. Bar 2 shows a sample prepared in the same way except that PEI-ss-PEG5000 was treated with 10 mM DTT before complexation.

FIG. 17 shows the Zeta potential of PEI and PEI-ss-PEG5000 complexed with salmon sperm DNA at a charge ratio of 3 (+/−).

FIG. 18 shows particle size stability of a cleavable PEI-ss-PEG5000/DNA complex containing 250 μg/ml Salmon sperm DNA; Charge ratio 1 (+/−),Mol % PEG in the complex: 10.0 Error bars represent the standard deviation of the particle size distribution

FIG. 19 shows luciferase activity of PEI/DNA and PEI-PEG and PEI-ss-PEG/DNA complexes. Cells (BL6) were transfected in serum free medium for 3 hours with 0.5 μg/well (in 96 well plate) of plasmid DNA complexed with PEI, PEI-PEG and PEI-ss-PEG at a charge ratio of 5. Luciferase activity was assayed 24 hours after transfection.

FIG. 20 shows luciferase activity of PEI/DNA and PEI-PEG/DNA complexes. Cells (BL6) were transfected in serum free medium for 3 hours with 0.5 μg/well (in 96 well plate) of plasmid DNA complexed with PEI or PEI-PEG at a charge ratio of 5. Luciferase activity was assayed 24 hours after transfection.

FIG. 21 shows the effect of PEG on the surface properties of the complex.

FIG. 22 shows the effect of PMOZ on the surface properties of the complex. The complexes were formulated at a charge-ratio of 4:1 and the zeta-potential measured in 10 mM saline.

FIG. 23 shows the effect of PMOZ on serum stability (4:1 charge ratio complexes were prepared with varying amounts of PMOZ from 0 to 3.2% (in steps of 0.8) and investigated for particle-size, before and after a 2 h incubation in PBS containing 10% FBS at 37° C.).

FIG. 24 shows the effect of PMOZ on the expression by PEI core complexes.

FIG. 25 shows increased expression by addition of a peptide ligand (K14RGD) to lipofectin core complexes.

FIG. 26 shows increased expression by addition of a peptide ligand (SMT or Somatostatin) to core complexes.

FIG. 27A shows synthesis of linear PEI conjugated with a hindered disulfide to polyethyloxazoline (PEOZ) at one end and to a peptide ligand, RGD, at the other end.

FIG. 27B shows synthesis of linear PEI conjugated with a hindered disulfide to polyethyloxazoline (PEOZ) at one end and to a peptide ligand, SMT, at the other end

FIG. 28 shows increased cellular uptake of Rh-oligonucleotides complexed with PEI by addition of a peptide ligand (RGD) to the distal end of PEG Conjugated PEI in HELA cells at charge ratio 6.

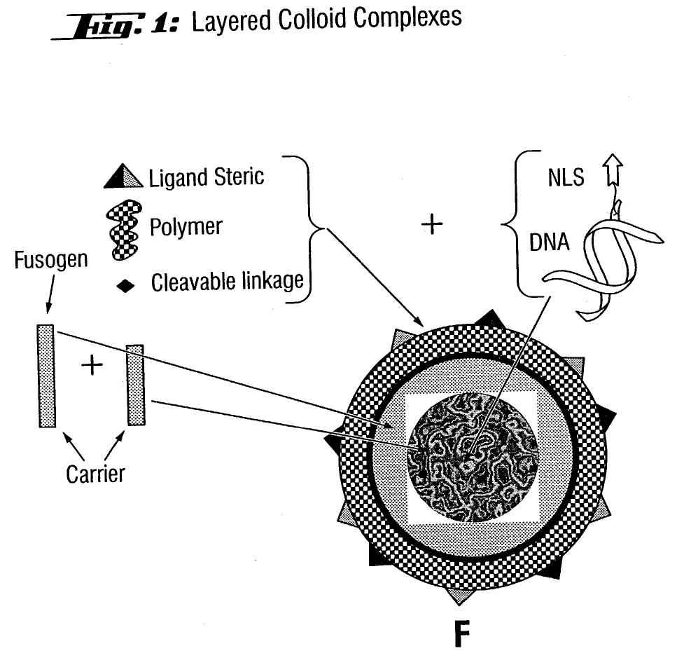

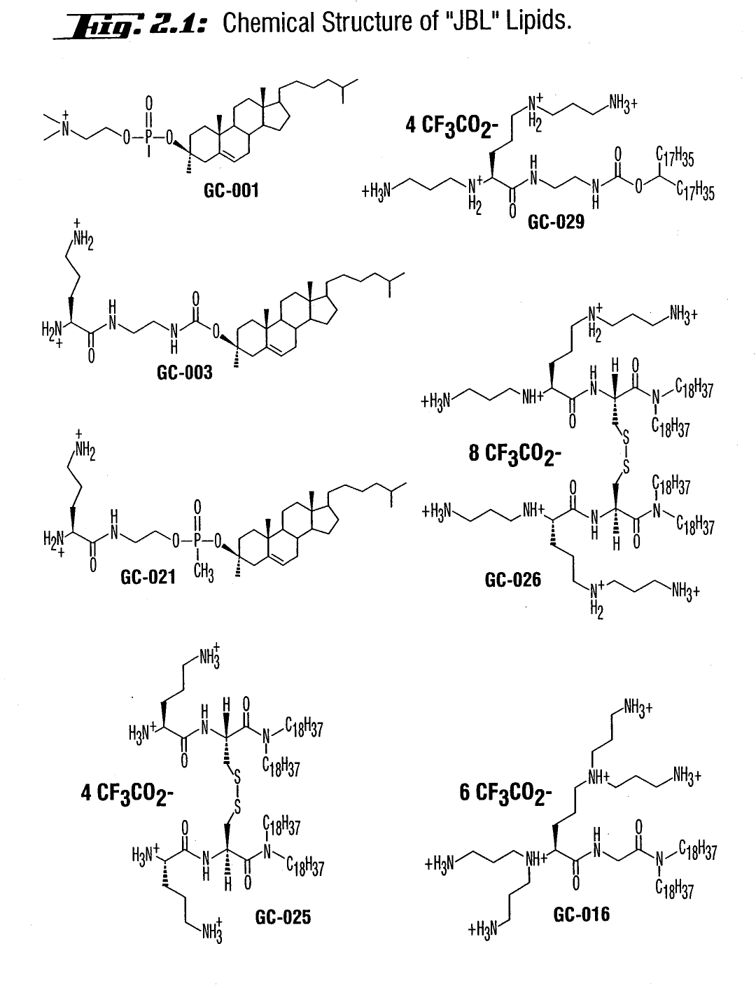

### DETAILED DESCRIPTION

Improved compositions and methods for delivery of therapeutic nucleic acid are provided. The improved complexes comprise a stable gene delivery vector having 1) an inner gene core complex and 2) an outer shell moiety anchored to the inner core complex. The outer shell moiety provides improved delivery of the nucleic acid, target specificity, in vivo biological stability, and colloidal or physical stability. The gene core complex contains a “payload” nucleic acid moiety, at least one core complex forming reagent, and advantageously contains additional functional units that facilitate cell entry, nuclear targeting, and nuclear entry of the nucleic acid moiety following entry into the target tissues and cell. The core complex is one in which the nucleic acid is localized in a compartment largely free of “bulk water”. Thus, the core complex is distinct from compositions such as liposomes that entrap a relatively dilute solution of nucleic acid and where the nucleic acid “floats” around inside. The core complex does contain many water molecules that hydrate the nucleci acid, but there is not a large “entrapped” volume as is found in a liposome.

The gene core complex may include a fusogenic moiety as an integral part of the core complex, or the fusogenic moiety may comprise a separate layer or shell of the vector. In this latter embodiment, the fusogenic moiety is anchored to the core complex, where the anchor comprises a linkage that is covalent, electrostatic, hydrophobic, or a combination of such forces. The nature of the anchoring linkage between the core complex and the fusogenic layer is such that the anchor may be separated from the nucleic acid once the vector enters the cytoplasm of the target cell, thereby enhancing the biological activity of the payload nucleic acid. Likewise the core complex forming reagent is such that the nucleic acid is released and free to exert its biological activity in the nucleus or other compartment of the cell where it exhibits its desired activity.

The nucleic acid moiety payload contains one or more DNA or RNA molecules or chemical analogues. In one embodiment, this moiety encodes a therapeutic peptide, polypeptide, or protein. The payload also may directly or indirectly inhibit expression of an endogenous gene in the target tissue and cell. For example, the payload may be a DNA molecule encoding a therapeutic RNA molecule or an antisense RNA, or may be an antisense oligonucleotide, a ribozyme, a double stranded RNA that inhibits gene expression, a double stranded RNA/DNA hybrid, a viral genome, or other forms of nucleic acids.

The functional unit that facilitates nuclear targeting of the nucleic acid following entry into the target tissue and cell advantageously is a nuclear localization signal. The skilled artisan will recognize, however, that other moieties may be used that enhance delivery of the core complex to the nucleus of the target tissue and cell. For example, the functional unit also may be a viral core peptide, polypeptide, or protein that enhances nuclear delivery, or may be a nuclear membrane transport peptide also known as nuclear localization signal (NLS), or a steroid or steroid analogue moiety (see Ceppi et al., Program of the American Society of Gene Therapy meeting held at Washington D.C. on June 9-13, p217a, abs# 860 (1999)).

In one embodiment, the gene delivery vector has a steric barrier outer layer or shell that provides modified surface characteristics for the complex, thereby diminishing the non-specific interactions that cause significant problems with conventional vector systems. The steric layer also has the advantage of suppressing the host immune response against the vector upon administration to the host. Advantageously, the outer layer protects the complex only prior to attachment and entry into the target tissue and cell. In one embodiment, the outer layer then is shed, allowing optimal biological activity of the payload nucleic acid. To achieve this goal, there is provided a steric coating on the surface of the complex, which minimizes interactions with serum components and non-target tissues and cells. The coating is anchored to the core complex in such a fashion that the steric coating is shed or cleaved from the complex at a point where cellular interactions are beneficial. For example, one such point may occur after attachment of the complex to the target tissue and cell, but prior to release of the core complex into the cell cytoplasm. Another such point is within the extracellular space of a target tissue. Yet another such point is after a predetermined time. Yet aother such point is within a target tissue that is exposed to an external signal or force such as heat or sonic energy. The sequence of events following cell entry ensures that delivery of the payload is not impeded or otherwise inhibited by the steric layer. In another embodiment, the steric layer is designed and anchored such that it inhibits non-specific interactions but permits binding to target tissues and cells, cell entry, and functional delivery of the nucleic acid without cleavage of the anchor.

The outer layer advantageously contains a targeting moiety that enhances the affinity of the interaction between the vector and the target tissue and cell. A targeting moiety is said to enhance the affinity of the vector for a target cell population when the presence of the targeting moiety provides an increase in the vector bound at the surface of target tissues and cells compared to non-target tissues and cells. Examples of targeting moieties include, but are not limited to proteins, peptides, lectins (carbohydrates), and small molecule ligands, where each of the targeting moieties binds to a complementary molecule or structure on the cell, such as a receptor molecule.

Particular features of the invention are described in detail below.

The Payload Nucleic Acid Moiety

The vectors of the present invention may be used to deliver essentially any nucleic acid that is of therapeutic or diagnostic value. The nucleic acid may be a DNA, an RNA, a nucleic acid homolog, such as a triplex forming oligonucleotide or peptide nucleic acid (PNA), or may be combinations of these. Suitable nucleic acids may include, but are not limited to, a recombinant plasmid, a replication-deficient plasmid, a mini-plasmid lacking bacterial sequences, a recombinant viral genome, a linear nucleic acid fragment encoding a therapeutic peptide or protein, a hybrid DNA/RNA double strand, double stranded RNA, an antisense DNA or chemical analogue, an antisense RNA or chemical analogue, a linear polynucleotide that is transcribed as an antisense RNA or a ribozyme, a ribozyme, and a viral genome. It will be understood that, as hereinafter used, the term “therapeutic protein” includes peptides, polypeptides, and proteins, unless otherwise indicated.

When it is desired that the nucleic acid be integrated site-specifically into the genome of the host cell, the nucleic acid sequence encoding the therapeutic protein may be flanked by stretches of sequence that are homologous to sequences in the host genome. These sequences facilitate integration into the host genome by the process of homologous recombination. Vectors for use in achieving homologous recombination are known in the art. When the nucleic acid is integrated in this site specific manner into the host genome, it is possible that expression of the nucleic acid can be under the functional control of endogenous expression control systems. More likely, however, it will be necessary to provide exogenous control elements that drive nucleic acid expression. Advantageously, the control elements will be cell-specific, thereby enhancing the cell-specific nature of the nucleic acid expression, though this is not essential. Suitable expression control elements, such as promoters and enhancer sequences (both cell-specific and non-specific) are well known in the art. See for example, Gazit et al., Can. Res. 59, 3100-3106 (1991), Walton et al., Anticancer Res, 18(3A): 1357-60 (1998); Clary et al., Surg-Oncol-Clin-N-Am. 7:565-74 (1998), Rossi et al./Curr-Opin-Biotechnol. 9: 451-6 (1998), Miller et al., Hum-Gene-Ther. 8:803 (1997); Clackson, Curr. Opin. Chem. Biol. 1:210-218 (1997). Suitable promoters include, but are not limited to, constitutive promotors such as EF-1a, CMV, RSV, and SV40 large T antigen promoters, tissue specific promoters such as albumin, lung surfactant protein, tissue specific growth factor receptors, pathological tissue specific promoters such as alfa fetal protein tumor specific promoters, tumor specific proteins, inflammatory cascade proteins, necrosis response proteins, regulated promoters such as tetracycline activated promoters and steroid receptor activated promoter or engineered promoters, and chromatin elements such as scaffold or matrix attachment regions (SAR or MAR), nucleosome elements, insulators, and enhancers.

Suitable expression plasmids and mini-plasmids for use in the invention are well known in the art (Prazeres et al., Trends-Biotechnol. 17:169 (1999); Kowalczyk et al., Cell-Mol-Life-Sci. 55:751 (1999); Mahfoudi, Gene Ther. Mol. Biol. 2:431 (1998). The plasmid may comprise an open reading frame sequence operationally coupled with promoter elements, intron sequences, and poly adenylation signal sequences. When the nucleic acid moiety is a plasmid, it advantageously will lack the nucleic acid elements that permit replication in bacteria. Thus, for example, the plasmid will lack a bacterial origin of replication. Most advantageously, the plasmid will be relatively free of sequences of bacterial origin. Methods for preparing such plasmids are well known in the art (Prazeres supra).

When the nucleic acid is of viral origin, suitable viral moieties include, but are not limited to, a recombinant adenoviral genome DNA (with and without the terminal protein), and a retroviral core derived from, for example, MLV or HIV env⁻ particles. A recombinant alpha virus RNA for cytoplasmic expression and replication also may be used. Other viral genomes include herpes virus, SV-40, vaccinia virus, and adeno associated virus. Plasmid DNA or PCR generated DNA encoding a viral genome may be used. Other viral sources of nucleic acid may be used.

When the nucleic acid is of synthetic origin, suitable moieties include, but are not limited to, PCR fragment DNA, DNA with terminal group chemical modifications or conjugation, antisense and ribozyme oligonucleotides, linear RNA, linear RNA-DNA hybrids. Other sources of synthetic nucleic acid or nucleic acid analogues may be used.

The Complex Forming Reagent

A complex-forming reagent suitable for use in the present invention must be capable of associating with the core nucleic acid in a manner that allows assembly of the nucleic acid core complex. The complex forming reagent may be, for example, a lipid, a synthetic polymer, a natural polymer, a semi-synthetic polymer, a mixture of lipids, a mixture of polymers, a lipid and polymer combination, or a spermine analogue complex, though the skilled artisan will recognize that other reagents may be used. The complex forming reagent preferably has an affinity sufficient to enable formation of the complex under the conditions present for the preparation and sufficient to maintain the complex during storage and under conditions present following administration but which is insufficient to maintain the complex under conditions in the cytoplasm or nucleus of the target cell. Common examples of complex-forming reagents include cationie lipids and polymers, which permit spontaneous complexation with the core nucleic acid moiety under suitable mixing conditions, although neutral and negatively charged lipids and polymers may be used. Other examples include lipids and polymers in combination where some are cationic in nature and others in the combination are neutral or anionic in nature such that together a complex with a desired stability balance is attained. In yet other examples, lipid and polymers may be used that have non-electrostatic interactions but that still enable complex formation with a desired stability balance. For example, the desired stability balance may be achieved through interactions with nucleic acid bases and back bone moieties like those of triplex oligonucletide or “peptide nucleic acid” binding. In yet further examples conjugated lipids and polymers alone and in combinations may be used.

Suitable cationic lipids for use in the invention are described, for example, in U.S. Pat. Nos. 5,854,224 and 5,877,220, which are hereby incorporated by reference in their entirety. Suitable lipids typically contain at least one hydrophobic moiety and one hydrophilic moiety. Other suitable lipids include a vesicle forming or vesicle compatible lipid, such as a phospholipid, a glycolipid, a sterol, or a fatty acid. Included in this class are phospholipids, such as phosphatidylcholine (PC), phosphatidylethanolamine (PE), phosphatidic acid (PA), phosphatidylglycerol (PG), phosphatidylinositol (PI), and glycolipids, such as sphingomyelin (SM), where these compounds typically contain two hydrocarbon chains that are characteristically between about 14-22 carbon atoms in length, and may contain unsaturated carbon-carbon bonds. One class of preferred hydrophobic moieties includes hydrocarbon chains and sterols. Other classes of hydrophobic moieties include sphingosine, ceramide, and terpenes (poly-isoprenes) such as farnesol, limonene, phytol, squalene, and retinol. Specific examples of lipids suitable for the invention include anionic, neutral, or zwitterionic lipids such as phosphatidylethanolamine, dioleoylphosphatidylethanolamine (DOPE), or cholesterol(Chol), cholesterol hemisuccinate (CHEMS), cholesterol sulfate, and diacylglycerol. Specific examples of cationic lipids include N-1-(2,3-dioleyloxy)propyl-N,N,N-trimethylammonium chloride (DOTMA), 1,2-bis (oleoyloxy)-3-(trimethylammonia) propane (DOTAP), 1, 2-dioleoyl-3-dimethylammonium propanediol (DODAP), dioctadecyldimethylammonium bromide (DODAB), dioctadecyldimethylammonium chloride (DODAC), dioctadecylamidoglycylspermine (DOGS), 1,3-dioleoyloxy-2-(6-carboxyspermyl)propylamide (DOSPER), 2,3-dioleyloxy-N-[2-(sperminecarboxamido)ethyl]-N,N-dimethyl-1-propanaminium trifluoroacetate (DOSPA or Lipfectamine™), hexadecyltrimethyl-ammonium bromide (CTAB), dimethyl-dioctadecylammonium bromide (DDAB), 1,2-dimyristyloxypropyl-3-dimethyl-hydroxy ethyl ammonium bromide (DMRIE), dipalmitoylphosphatidylethanolamylspermine (DPPES), dioctylamineglycine-spermine (C8Gly-Sper), dihexadecylamine-spermine (C18-2-Sper), aminocholesterol-spermine (Sper-Chol), 1-[2-(9(Z)-octadecenoyloxy)ethyl]-2-(8(Z)-heptadecenyl)-3-(2-hydroxyethyl)imidazolinium chloride (DOTIM), dimyristoyl-3-trimethylammonium-propane (DMTAP), 1.2-dimyristoyl-sn-glycero-3-ethylphosphatidylcholine (EDMPC or DMEPC), lysylphosphatidylethanolamine (Lys-PE), cholestryl-4-aminoproprionate (AE-Chol), spermadine cholestryl carbamate (Genzyme-67), 2-(dipalmitoyl-1,2-propandiol)-4-methylimidazole (DPIm), 2-(dioleoyl-1,2-propandiol)-4-methylimidazole (DOIm), 2-(cholestryl-1-propylamine carbamate)imidazole (ChIm), N-(4-pyridyl)-dipalmitoyl-1,2-propandiol-3-amine (DPAPy), 3β-[N—(N′,N′-dimethylaminoethane)carbamoyl]cholesterol (DC-Chol), 3β-[N—(N′,N′,N′-trimethylaminoethane)carbamoyl] cholesterol (TC-CHOL-gamma-d3), 1:1 mixture of DOTMA and DOPE (Lipofectin 7), 1,2-dioleoyl-sn-glycero-3-succinate, 1,2-dioleoyl-sn-glycero-3-succinyl-2-hydroxethyl disulfide ornithine conjugate (DOGSDSO) and 1,2-dioleoyl-sn-glycero-3-succinyl-2-hydroxethyl hexyl orithine conjugate (DOGSHDO), N,N^{I},N^{II},N^{III}-tetramethyl-N,N^{I},N^{II},N^{III}-tetrapalmityolspermine (TM-TPS), 3-tetradecylamino-N-tert-butyl-N′-tetradecylpropionamidine (vectamidine or diC14-amidine), N-[3-[2-(1,3-dioleoyloxy)propoxy-carbonyl]propyl]-N,N,N-trimethylammonium iodide (YKS-220), and O,O′-Ditetradecanoyl-N-(alpha-trimethylammonioacetyl)diethan olamine chloride (DC-6-14) (see Lasic, Liposomes in Gene Delivery, 1997, CRC Press, Boca Raton Fla., Tang et al., Biochem. Biophys. Res. Comm. 242:141 (1998); Obika et al., Biol-Pharm-Bull. 22:187 (1999).

Note that mixtures of a cationic lipid with a neutral lipid can be used, as well as mixtures of cationic lipids plus neutral lipids including 3:1 wt/wt DOSPA:DOPE (Lipofectamine 7), 1:1 wt/wt DOTMA:DOPE (Lipofectin 7), 1:1 Mole/Mole DMRIE:Chol (DMRIE-CTM), 1:1.5 Mole/Mole TM-TPS:DOPE (Cellfectin™), 1:2.5 wt/wt DDAB:DOPE (LipofectACE7), 1:1 wt/wt DOTAP:Chol, and many variants on these.

Also note that such cationic lipid reagents, as well as other cationic reagents that lack the hydrophobic moiety, can bind to the nucleic acid in such a manner that the nucleic acid is incorporated into low polarity environments including oils formed with triglyceride and/or sterols, emulsions formed with oils combined with amphipathic stabilizers such as fatty acids and lysophospholipids, microemulsions, an cubic phase lipid. One specific embodiment utilizes a multivalent cationic lipid such as DOGS in combination with with triglyceride and phosphatidylcholine:lysophosphatidylcholine (2:1 or other ratio as needed to control particle size). Such compositions can be used to form core particles where anchoring occurs via addition of large hydrophobic moieties (having very low water solubility) such as octyldecyl (C₁₈) and longer hydrocarbon, phytanoyl hydrocarbon, or multiple moieties, or other such moieties. Another specific embodiment utilizes a multivalent cationic lipid such as DOGS in combination with hydrocarbon-flurocarbon “dowel” (C₁₆F₁₇H₁₇), fluorocarbon “oil” (e.g. C₁₆F₃₄), and phosphatidylcholine:-lysophosphatidylcholine (2:1 or other ratio as needed to control particle size). Such compositions can be used to form core particles where anchoring is by addition of fluorocarbon or hydrocarbon-fluorocarbon segments which can insert into the fluorcarbon “oil”.

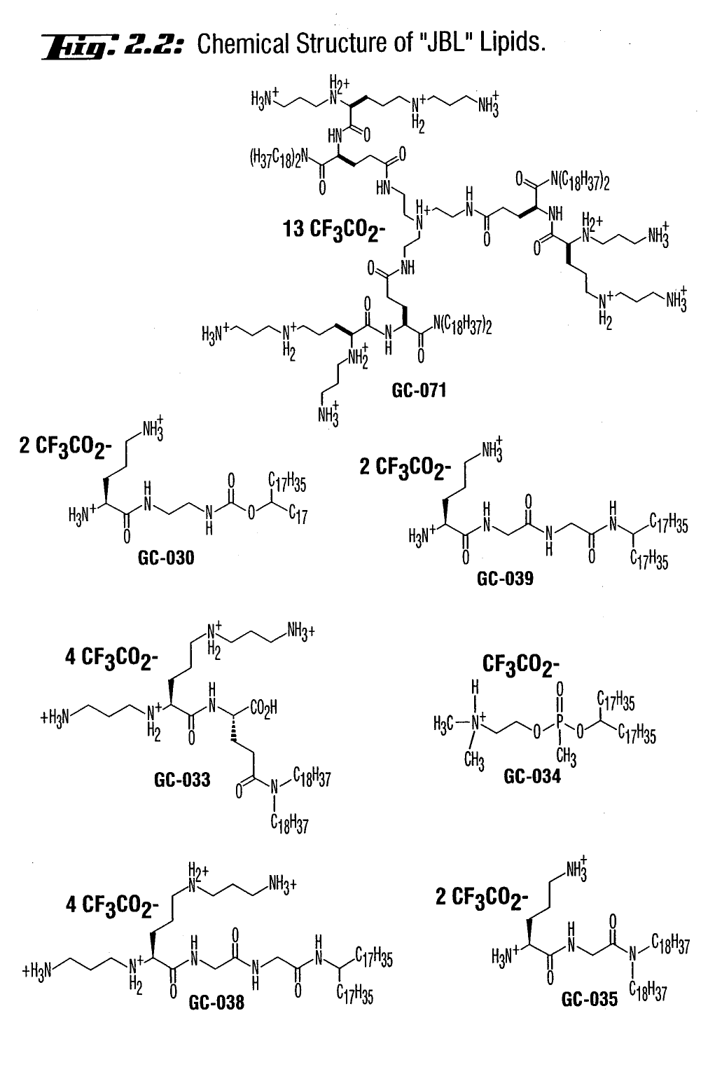

A number of other cationic lipids are suitable for forming the core complex, and are described in the following patents or patent applications: U.S. Pat. Nos. 5,264,618, 5,334,761, 5,459,127, 5,705,693, 5,777,153, 5,830,430, 5,877,220, 5,958,901, 5,980,935, WO 09640725, WO 09640726, WO 09640963, WO 09703939, WO 09731934, WO 09834648, WO 9856423, WO 09934835. For example, fourteen reagents described by patents or patent applications U.S. Pat. Nos. 5,877,220, 5,958,901, WO 96/40725, WO 96/40726, and WO 97/03939 are commercially available from Promega Biosciences [formerly JBL Scientific subsidiary of Genta Inc.] (San Louis Obisbo, Calif.) and their structures are shown in FIGS. 2.1-2.2. The hydrophobic portions range from sterol (cholesterol) to two or four hydrocarbon chains 17 or 18 carbons in length. The positively charged portions (hydrophilic head groups) vary greatly but generally contain ionizable nitrogens (amines). The number of positive charges on each molecule varies from 1 to 13 and the molecular weight varies from 650 to 4212.

Advantageously, the core complex can be prepared with GC-030 or GC-034, either without any accessory components or with accessory components such as cholesterol or surfactants containing hydrophilic polymer moieties. Alternatively, GC-029, GC-039, GC-016, GC-038 can be used, either alone or as mixtures with components such as Chol or surfactants. Numerous other lipid structures are described in U.S. Pat. Nos. 5,877,220, 5,958,901, WO 96/40725, WO 96/40726, and WO 97/03939 and may be used in the invention. The specific lipids having greatest utility can be identified using four kinds of assays: 1) ability to form the nucleic acid into small, colloidally stable, particles, 2) ability to enhance internalization of the nucleic acid into endosomes in cells in tissue culture, 3) ability to enhance cytoplasmic release of the nucleic acid in cells in tissue culture, and 4) ability to elicit plasmid expression by in vivo tissues when administered locally or systemically.

Suitable cationic compounds further include substituted aminoethanols, having the general formula I

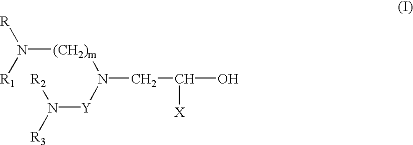

where m is 3 or 4; Y signifies a group —(CH₂)ₙ—, in which n is 3 or 4, or may also signify a group —(CH₂)ₙ—, in which n is an integer from 5 to 16, or may also signify a group —CH₂—CH═CH—CH₂—, if R₂ is a group —(CH₂)₃—NR₄R₅ and m is 3; R₂ is hydrogen or lower alkyl or may also signify a group —(CH₂)₃—NR₄R₅ if m is 3; R₃ is hydrogen or alkyl or may also signify a group —CH₂—CH(—X′)—OH, if R₂ is a group —(CH₂)₃—NR₄R₅ and m is 3; X and X′, independently of one another, signify hydrogen or alkyl; and the radicals R, R₁, R₄ and R₅, independently of one another, are hydrogen or lower alkyl; with the proviso that the radicals R, R₁, R₂, R₃ and X cannot all together signify hydrogen or methyl, if m is 3 and Y signifies a group —(CH₂)₃—; and their salts.

The general terms used hereinbefore and hereinafter have the following significances in the context of the present application:

The prefix “lower” indicates a radical with up to and including 7, and in particular up to and including 3, carbon atoms.

Lower alkyl is, for example, n-propyl, isopropyl, n-butyl, isobutyl, sec.-butyl, tert.-butyl, n-pentyl, neopentyl, n-hexyl or n-heptyl. In one embodiment, lower alkyl is preferably ethyl and in particular methyl. In another embodiment, lower alkyl is fluorocarbon analogues of the hydrocarbon moieties. In yet another embodiment, lower alkyl is a combination of fluorocarbon and hydrocarbon.

Alkyl is, for example, C₁-C₃₀-alkyl, preferably C₁-C₁₆-alkyl; alkyl is preferably linear alkyl, but may also be branched and is, for example, lower alkyl as defined above, n-octyl, n-nonyl, n-decyl, n-dodecyl, n-tetradecyl, n-hexadecyl or 2,7-dimethyloctyl. In another embodiment, alkyl is fluorocarbon analogues of the hydrocarbon moieties. In yet another embodiment, alkyl is a combination of fluorocarbon and hydrocarbon.

Halogen signifies, for example, fluorine or iodine, especially bromine and in particular chlorine.

Salts of compounds according to the invention are primarily pharmaceutically acceptable, non-toxic salts. For example, compounds of formula I that contain either 3 or 4 basic centres may form acid addition salts e.g. with inorganic acids, such as halogen acids like hydrochloric and hydroiodic acid, with sulfuric acid or phosphoric acid, or with appropriate organic carboxylic acids or sulfonic acids, e.g. acetic acid, trifluoroacetic acid, fumaric acid, oxalic acid, methanesulfonic acid or p-toluenesulfonic acid, or e.g. with acidic amino acids, such as aspartic acid or glutamic acid. When associated with compounds of formula I, the term “salts” includes both monosalts and polysalts.

For isolation or purification, pharmaceutically unsuitable salts may also be used, e.g. picrates or perchlorates. For therapeutical usage, only the pharmaceutically acceptable salts may be used, and for this reason these are preferred.

Depending on the structural data, the compounds of the present invention may exist in the form of isomeric mixtures or as pure isomers.

The compounds of formula I may be produced in known manner, whereby e.g.

(a) a compound of formula II

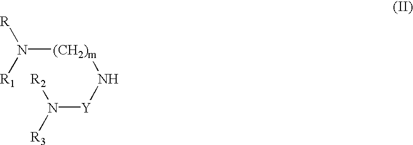

wherein m, Y, R, R₁, R₂ and R₃ are defined as for formula I, in which the amino groups —NRR₁, —NR₂R₃ and optionally —NR₄R₅ in a radical R₂=—(CH₂)₃—NR₄R₅ are optionally protected by appropriate protecting groups, is reacted with a compound of formula III

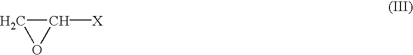

where X is defined as for formula I, and if necessary, the amino protecting group(s) are cleaved again, or

(b) in order to produce compounds of formula I, in which m is 3, R₂ is a group —(CH₂)₃—NR₄R₅ and R₃ is a group —CH₂—CH(—X′)—OH, a compound of formula IV

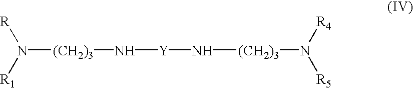

wherein Y, R, R₁, R₄ and R₅ are defined as for formula I, and in which the amino groups —NRR₁ and —NR₄R₅ are optionally protected by appropriate protecting groups, is reacted with a compound of formula III, in which X is defined as for formula I, and if necessary, the amino protecting group(s) are cleaved again, or

(c) in order to produce compounds of formula I, wherein R, R₁, R₂ and R₃ signify hydrogen and Y is a group —(CH₂)ₙ—, in which n is 3 or 4, a compound of formula V

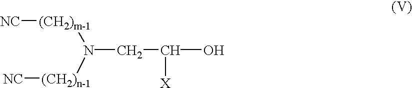

wherein m and X are defined as for formula I and n is 3 or 4, is reduced, or

(d) in order to produce compounds of formula I, in which m is 3, R₂ signifies a group —(CH₂)₃—NH₂ and R and R₁ signify hydrogen, a compound of formula VI

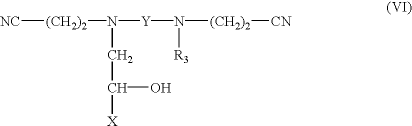

wherein X, Y and R₃ are defined as for formula I, is reduced; and/or if desired, an obtained compound of formula I may be converted into another compound of formula I, and/or, if desired, an obtained salt may be converted into the free compound or into another salt, and/or, if desired, an obtained free compound of formula I with salt-forming properties may be converted into a salt, and/or an obtained mixture of isomeric compounds of formula I may be separated into the individual isomers.

In the more detailed description of processes a) to d) that follows, the symbols m, n, X, X′, Y, R and R₁ to R₅ have the significances given for formula I, unless stated otherwise.

Process (a): The amino groups —NRR₁, —NR₂R₃ and optionally —NR₄R₅ are preferably protected by protecting groups. The way in which protecting groups act, e.g. amino protecting groups, the introduction thereof and cleavage thereof are known per se and are described e.g. in J. F. W. McOmie, “Protecting Groups in Organic Chemistry”, Plenum Press, London and New York 1973, and T. W. Greene, “Protecting Groups in Organic Synthesis”, Wiley, New York 1984. Amino protecting groups that are especially suitable for polyamines such as spermine, spermidine, etc., are described e.g. in Acc. Chem. Res. 19:105 (1986) and Z. Naturforsch. 41b, 122 (1986).

Preferred monovalent amino protecting groups are ester groups, e.g. lower alkyl esters and in particular tert.-butoxycarbonyl (BOC), or phenyl lower alkyl esters, e.g. benzyloxycarbonyl (carbobenzoxy, Cbz), or acyl radicals, e.g. lower alkanoyl or halogen lower alkanoyl, such as especially acetyl, chloroacetyl or trifluoroacetyl, or sulfonyl radicals, e.g. methylsulfonyl, phenylsulfonyl or toluene-4-sulfonyl. Preferred bivalent amino protecting groups are bisacyl radicals, e.g. that of phthalic acid (phthaloyl), which together with the nitrogen atom to be protected forms a phthalimido group.

Cleavage of the amino protecting groups may take place e.g. hydrolytically, perhaps in an acidic medium, e.g. with hydrochloric acid, or in an alkaline manner, e.g. with sodium hydroxide solution, or also by hydrogenation.

Tert.-butoxycarbonyl is particularly preferred as the amino protecting group, and may be introduced e.g. by reacting the free amines with 2-(tert.-butoxycarbonyloxyimino)-2-(phenylacetonitrile [tert.-butyl-O—C(═O)—O—N═C(-phenyl)-CN] or with di-(tert.-butyl)-dicarbonate. Cleavage of tert.-butoxycarbonyl is effected e.g. in an acidic medium, in particular with oxalic acid or oxalic acid dihydrate, hydrochloric acid or toluene-4-sulfonic acid or toluene-4-sulfonic acid monohydrate.

Likewise preferred as the amino protecting group is benzyloxycarbonyl, which may be introduced by reacting the free amines with chloroformic acid benzyl ester. Cleavage of the benzyloxycarbonyl is preferably effected by hydrogenation, e.g. in the presence of palladium on activated carbon.

Also preferred as the amino protecting group is toluene-4-sulfonyl, which may be introduced by reacting the free amines with toluene-4-sulfochloride, optionally employing an auxiliary base such as triethylamine. Cleavage of toluene-4-sulfonyl is preferably effected in an acidic medium, e.g. with concentrated sulfuric acid or 30% hydrobromic acid in glacial acetic acid and phenol, or also under alkaline conditions, e.g. with LiAlH₄.

Also preferred as the protecting group for terminal primary amino groups is phthaloyl, which is preferably introduced by a reaction with N-ethoxycarbonyl phthalimide. Cleavage of this protecting group takes place e.g. by reacting with hydrazine.

The starting compounds of formulae II and III are known or may be produced in analogous manner to known compounds. The compounds of formula II in question are, in particular, spermidine, homospermidine, norspermidine, spermine, dehydrospermine or N,N′-bis(3-aminopropyl)-α,ω-alkylenediamine [see e.g. J. Med. Chem. 7, 710 (1964)], which exist in free form or protected form, and derivatives thereof.

Compounds of formula III, wherein X signifies alkyl; may be present in racemic or optically active form. If they are used as pure enantiomers in the reaction according to process (a) [or (b)], the corresponding optically active compounds of formula I are obtained. Similarly, when reacted with compounds of formula VII or VIII [see below processes (c) and (d)] optically active compounds of formula V or VI are obtained.

The reaction according to process (a) may take place in the presence of a solvent or also without solvents.

Process (b): Process (b) corresponds to process (a), with the difference that here the group —CH₂—CH(—X or —X′)—OH is doubly introduced into the starting compounds of formula IV. Here also, the amino groups —NRR₁ and —NR₄R₅ are preferably protected by protecting groups.

The starting compounds of formula IV are known or may be produced in analogous manner to known compounds. The compounds of formula IV in question are, in particular, spermine, dehydrospermine or N,N′-bis(3-aminopropyl)-α,ω-alkylenediamine, which exist in free form or protected form, and derivatives thereof.

Process (c): The reduction according to process (c) may be effected e.g. with hydrogen in the presence of suitable catalysts, e.g. Raney nickel. In addition, reduction may also be carried out with complex metal hydrides, such as LiAlH₄ or NaBH₄. One preferred system for the reduction of compounds of formula V is H₂/Raney nickel in the presence of ethanol and ammonia or ethanol and sodium hydroxide.

The starting compounds of formula V may be obtained e.g. by reacting a compound of formula VII

NC—(CH₂)ₘ₋₁—NH—(CH₂)ₙ₋₁—CN (VII)

with a compound of formula III.

The compounds of formula VII are in turn obtainable e.g. by reacting ammonia with compounds of formula Hal-(CH₂)₂ or ₃—CN (Hal=halogen) [see C.A. 63, 2642b (1963) or J. Med. Chem. 15, 65 (1972)]. Unsymmetrical compounds of formula VII may be obtained e.g. according to C.A. 63, 2642b (1963) by reacting NC—(CH₂)₃—NH₂ with acrylonitrile.

Process (d): The reduction according to process (d) is carried out in the same way as that of process (c). The same reduction agents as in (c) are used.

The starting compounds of formula VI may be obtained e.g. by reacting a compound of formula VII

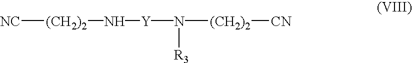

with a compound of formula III.

The compounds of formula VIII are in turn obtainable e.g. by reacting a diamine H₂N—Y—NHR₃ with acrylonitrile.

Compounds of formula I may be converted into other compounds of formula I in known manner. For example, compounds of formula I, wherein R, R₁ and R₂ and R₃ (or R₄ and R₅) signify hydrogen, may be lower alkylated by reacting with aldehydes or ketones, e.g. formaldehyde, under reductive conditions, e.g. with hydrogen in the presence of palladium on carbon, whereby for example compounds of formula I are obtained, wherein R, R₁ and R₂ and R₃ (or R₄ and R₅) signify lower alkyl. Furthermore, e.g. compounds of formula I, wherein m is 3, R₃ signifies hydrogen, R₂ is a group —(CH₂)₃—NR₄R₅ and the amino groups —NRR₁ and —NR₄R₅ are protected by protecting groups, may be reacted to form analogous compounds of formula I, wherein R₃ signifies alkyl, by reacting with alkylation agents, for example alkyl halides or dialkyl sulfates.

Free compounds of formula I having salt-forming properties, which are obtainable according to this process, may be converted in known manner into the salts thereof. Since the free compounds of formula I contain basic groups, they may be converted into the acid addition salts thereof by treating with acids.

Owing to the close relationship between the compounds of formula I in free form and in the form of salts, hereinbefore and hereinafter the free compounds or their salts are accordingly understood to mean also the corresponding salts or free compounds.

The compounds, including their salts, may also be obtained in the form of their hydrates, or their crystals may include e.g. the solvent used for crystallization.

Mixtures of isomers that are obtainable according to the invention can be separated in known manner into the individual isomers, racemates e.g. by forming salts with optically pure salt-forming reagents and separating the diastereoisomeric mixture thus obtainable, for example by fractional crystallization.

The above-mentioned reactions may be carried out under known reaction conditions, in the absence or normally in the presence of solvents or diluents, preferably those which are inert towards the reagents employed-and which dissolve them, in the absence of presence of catalysts, condensation agents or neutralising agents, depending on the type of reaction and/or the reaction components at reduced, normal or elevated temperature, e.g. in a temperature range of ca. −70° C. to 190° C., preferably −20° C. to 150° C., e.g. at boiling point of the solvent employed, under atmospheric pressure or in a closed container, optionally under pressure and/or in an inert atmosphere, e.g. under a nitrogen atmosphere.

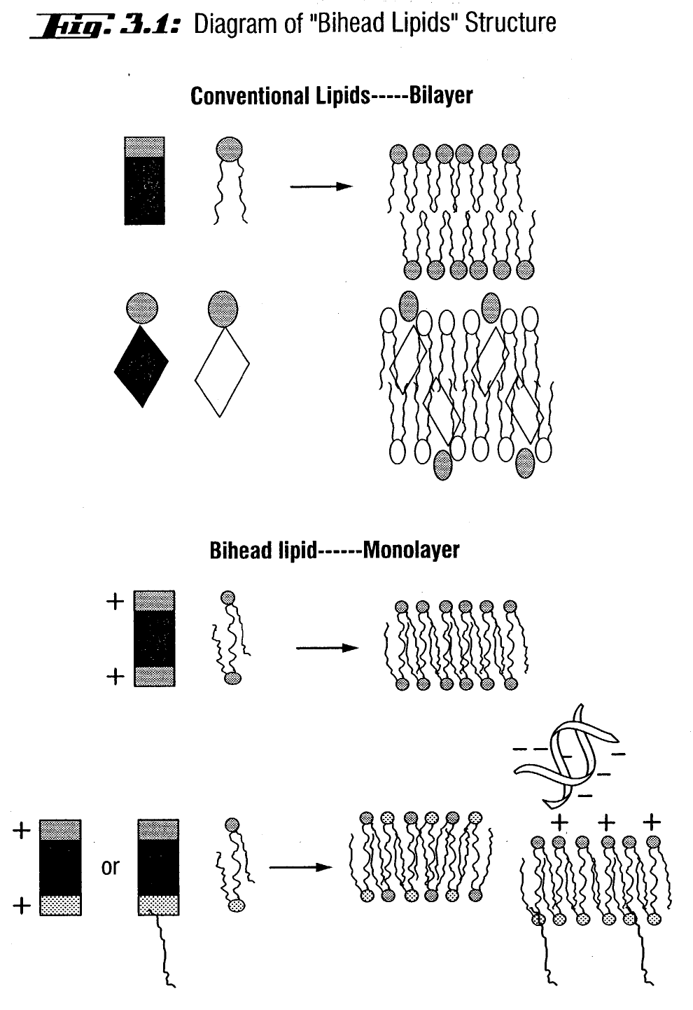

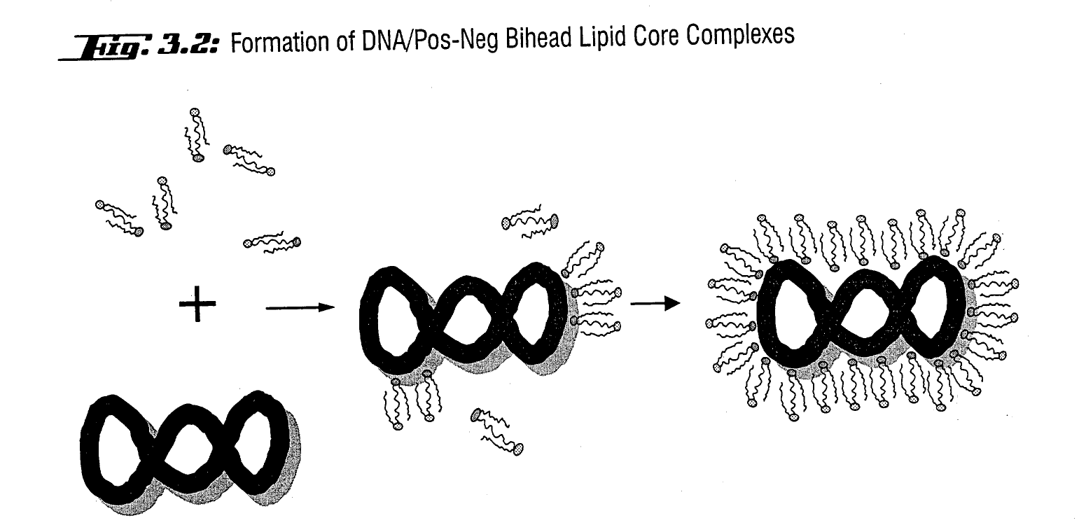

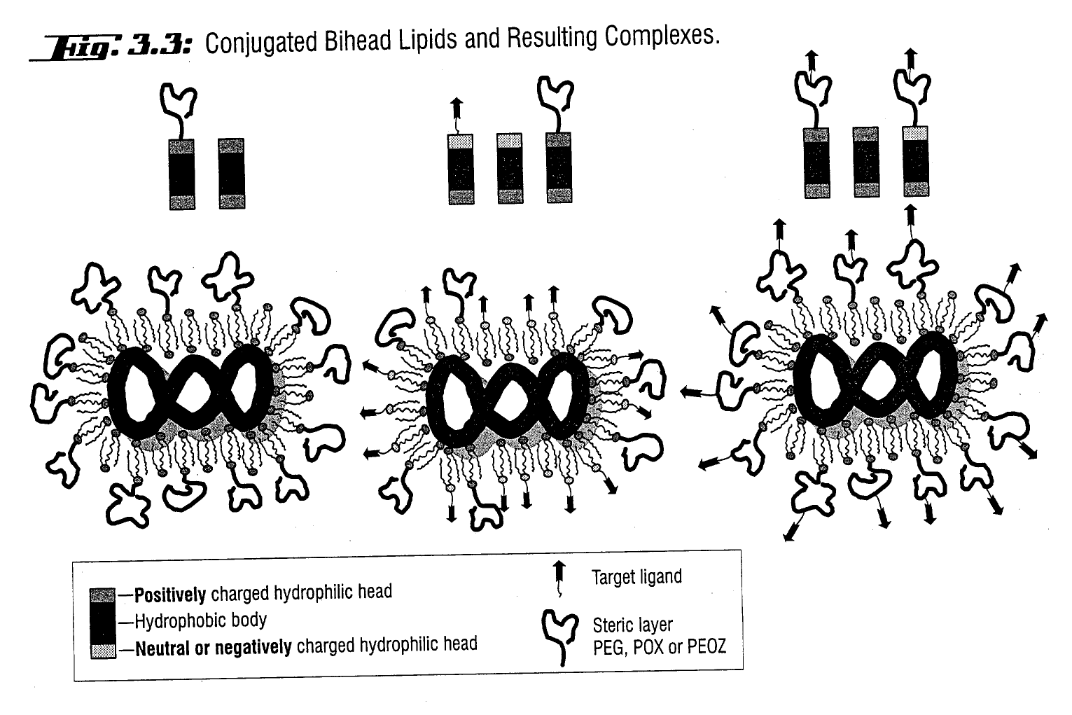

In some instances, substituted aminoethanols appear to have two hydrophilic polar heads connected by one hydrophobic body (FIG. 3) and are referred to as bihead lipids. Since two hydrophilic heads at either side can face an aqueous solution, these compounds can form a monolayer in water instead of a bilayer formed by lipids with one head group (FIG. 3).

In another embodiment of the substituted aminoethanols, bihead lipid forms other than those described above can be used where the substituted aminoethanols have different electrostatically charged polar heads, such as one positive and the other negative or neutral, and they can be used to form core complexes with a net excess of cationic charge in complexation with the nucleic acid but the complex formed has a neutral or negative surface charge. Such different polar bihead lipids can bind DNA with the positive head and form a monolayer coat around DNA with the negative or neutral head outside and thus a preferred negative or neutral surface charge. Further, the negative or neutral head provides a preferred moiety for anchoring other components of the vector. This is shown diagrammatically in FIGS. 3.1-3.4.

The two heads can have either the same or different charge states or forms that have substantially different pK values such as a primary amine and an imidazole. Preparation of bihead lipids with heads that have different charged states have unique properties. Bihead lipids having one positive head and the other negative or neutral permit the positive head to bind to nuclear acid and the negative or neutral head to form an exterior surface of the complex facing the aqueous solution (FIG. 3). Using the nucleic acid as a template for complex formation, the positive head binds and form a monolayer around it resulting in a monolayer liposome/nucleic acid complex an with anionic or neutral surface. Such bihead lipids can highly encapsulate plasmid DNA, other nucleic acids, or any negatively charged substances giving a negative or neutral surface charge which avoids adverse biological interactions such as those leading to toxicity.

The bihead lipids can be modified in other ways to give different properties of each head group. For example, one head can be conjugated with a steric polymer, with a targeting ligand, with a fusogenic moiety, or with combinations of moieties such as a steric polymer with a targeting ligand at the distal end. (FIG. 3).

The third kind of bihead lipids have both heads negative or neutral. These form useful monolayers of lipid around substances for control of pharmacokinetics and biodistribution much like liposomes and emulsions are used.

Suitable cationic compounds also include spermine analogues. The core complex formed with spermine analogues preferably comprises membrane disruption agents. In another embodiment, the core complex formed with spermine analogues comprises anionic agents to convey a negative surface charge to the core complex.

Suitable polymers for use in the invention include polyethyleneimine (PEI), and advantageously PEI that is linear, polylysine, polyamidoamine (PAMAM dendrimer polymers, U.S. Pat. No. 5,661,025), linear polyamidoamine (Hill et al., Linear poly(amidoamine)s: physicochemical interactions with DNA and Biological Properties, in Vector Targeting Strategies for Therapeutic Gene Delivery (Abstracts form Cold Spring Harbor Laboratory 1999 meeting), 1999, p 27), protamine sulfate, polybrine, chitosan (Leong et al. J Controlled Release 1998 Apr; 53(1-3):183-93), polymethacrylate, polyamines (U.S. Pat. No. 5,880,161) and spermine analogues (U.S. Pat. No. 5,783,178), polymethylacrylate and its derivatives such as poly[2-(diethylamino)ethyl methacrylate] (PDEAMA) (Asayama et al., Proc. Int. Symp. Control. Rel. Bioact. Mater. 26, #6236 (1999) and Cherng et al. Eur J Pharm Biopharm 47(3):215-24 (1999)) and poly(2-(dimethylamino)ethyl methacrylate) (PDMAEMA) (van de Wetering et al., J Controlled Release 53:145-53(1998)), poly(organo)phosphazenes (U.S. Pat. No. 5,914,231), which are hereby incorporated by reference in their entirety. Other polymers that may be used in the complex include polylysine, (poly(L), poly(D), and poly(D/L)), synthetic peptides containing amphipathic aminoacid sequences such as the “GALA” and “KALA” peptides (Wyman T B, Nicol F, Zelphati O, Scaria P V, Plank C, Szoka F C Jr, Biochemistry 1997, 36:3008-3017; Subbarao N K, Parente R A, Szoka F C Jr, Nadasdi L, Pongracz K, Biochemistry 1987 26:2964-2972) and forms containing non-natural aminoacids including D aminoacids and chemical analogues such as peptoids, imidazole-containing polymers, and fully synthetic polymers that bind and condense nucleic acid. Assays for polymers that exhibit such properties include measurements of plasmid DNA condensation into small particles using physical measurements such as DLS (dynamic light scattering) and electron microscopy.

Other reagents useful in the invention for a core forming reagent include polymers with the general structure:

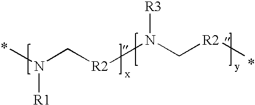

where R1 and R3 independently are a hydrocarbon or a hydrocarbon substituted with an amine, guanidinium, or imidazole moiety, and R2 is a lower alkyl group, or the general structure:

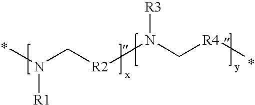

where R1 and R3 independently are a hydrocarbon or a hydrocarbon substituted with an amine, guanidinium, or imidazole moiety, and R2 and R4 independently are lower alkyl groups.

Further reagents useful in the invention for a core forming reagent include those with a mixture of cationic and anionic groups, and in some instances an excess of negative charges, such that the complex formed has a net negative charge. Examples of such reagents are those having the general structure:

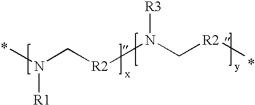

where R1 is a hydrocarbon or a hydrocarbon substituted with an amine, guanidinium, or imidazole moiety, R2 is a lower alkyl group, and R3 is a hydrocarbon or a hydrocarbon substituted with a carboxyl, hydroxyl, sulfate, or phosphate moiety; or reagents having the structure:

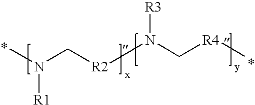

where R1 is a hydrocarbon or a hydrocarbon substituted with an amine, guanidinium, or imidazole moiety, R2 and R4 independently are lower alkyl groups, and R3 is a hydrocarbon or a hydrocarbon substituted with a carboxyl, hydroxyl, sulfate, or phosphate moiety.

Nuclear Targeting Moiety

A major barrier to efficient transcription and consequent expression of an exogenous nucleic acid moiety is the requirement that the nucleic acid enter the nucleus of the target cell.

Advantageously, when the intended biological activity of the nucleic acid payload is the nucleus, the nucleic acid of the invention is “nuclear targeted,” that is, it contains one or more molecules that facilitate entry of the nucleic acid through the nuclear membrane into the nucleus of the host cell, a nuclear localization signal (“NLS”). Such nuclear targeting may be achieved by incorporating a nuclear membrane transport peptide, or nuclear localization signal (“NLS”) peptide, or small molecule that provides the same NLS function, into the core complex. Suitable peptides are described in, for example, U.S. Pat. Nos. 5,795,587 and 5,670,347 and in patent application WO 9858955, which are hereby incorporated by reference in their entirety, and in Aronsohn et al., J. Drug Targeting 1:163 (1997); Zanta et al., Proc. Nat'l Acad. Sci. USA 96:91-96 (1999); Ciolina et al., Targeting of Plasmid DNA to Importin alpha by Chemical coupling with Nuclear Localization Signal Peptides, in Vector Targeting Strategies for Therapeutic Gene Delivery (Abstracts from Cold Spring Harbor Laboratory 1999 meeting), 1999, p 20; Saphire et al., J Biol Chem; 273:29764 (1999). A nuclear targeting peptide may be a nuclear localization signal peptide or nuclear membrane transport peptide and it may be comprised of natural aminoacids or non-natural aminoacids including D aminoacids and chemical analogues such as peptoids. The NLS may be comprised of aminoacids or their analogues in a natural sequence or in reverse sequence. Another embodiment is comprised of a steroid receptor-binding NLS moiety that activates nuclear transport of the receptor from the cytoplasm, where this transport carries the nucleic acid with the receptor into the nucleus (Ceppi supra).

In a further embodiment, the NLS is anchored onto the core complex in such a manner that the core complex is directed to the cell nucleus where it permits entry of the nucleic acid into the nucleus.

In one embodiment, incorporation of the NLS moiety into the vector occurs through association with the nucleic acid, and this association is retained within the cytoplasm. This minimizes loss of the NLS function due to dissociation with the nucleic acid and ensures that a high level of the nucleic acid is delivered to the nucleus. Furthermore, the association with the nucleic acid does not inhibit the intended biological activity within the nucleus once the nucleic acid is delivered.

In yet another embodiment, the intended target of the biological activity of the nucleic acid payload is the cytoplasm or an organelle in the cytoplasm such as ribosomes, the golgi apparatus, or the endoplasmic reticulum. In this embodiment, a localization signal is included in the core complex or anchored to it so that it provides direction of the nucleic acid to the intended site where the nucleic acid exerts its activity. Signal peptides that can achieve such targeting are known in the art.

Fusogenic Moiety

The fusogenic layer promotes fusion of the vector to the cell membrane of the target cell, facilitating entry of the nucleic acid payload into the cell. As described above, the fusogenic moiety may be incorporated directly into the core complex itself, or may be anchored to the core complex. In one embodiment, the fusogenic layer comprises a fusion-promoting element. Such elements interact with cell membranes or endosome membranes in a manner that allows transmembrane movement of large molecules or particles or that disrupts the membranes such that the aqueous phases that are separated by the membranes may freely mix. Examples of suitable fusogenic moieties include membrane surfactant peptides e.g. viral fusion proteins such as hemagglutinin (HA) of influenza virus, or peptides derived from toxins such as PE and ricin. Other examples include sequences that permit cellular trafficking such as HIV TAT protein and antennapedia or those derived from numerous other species, or synthetic polymers that exhibit pH sensitive properties such as poly(ethylacrylic acid) (Lackey et al., Proc. Int. Symp. Control. Rel. Bioact. Mater. 1999, 26, #6245), N-isopropylacrylamide methacrylic acid copolymers (Meyer et al., FEBS Lett. 421:61 (1999)), or poly(amidoamine)s, (Richardson et al., Proc. Int. Symp. Control. Rel. Bioact. Mater. 1999, 26, #251), and lipidic agents that are released into the aqueous phase upon binding to the target cell or endosome. Suitable membrane surfactant peptides include an influenza hemagglutinin or a viral fusogenic peptide such as the Moloney murine leukemia virus (“MoMuLV” or MLV) envelope (env) protein or vesicular stroma virus (VSV) G-protein.

Advantageously, the membrane-proximal cytoplasmic domain of the MoMuLV env protein may be used. This domain is conserved among a variety viruses and contains a membrane-induced α-helix.

Suitable viral fusogenic peptides for the instant invention include a fusion peptide from a viral envelope protein ectodomain, a membrane-destabilizing peptide of a viral envelope protein membrane-proximal domain, hydrophobic domain peptide segments of so called viral “fusion” proteins, and an amphiphilic-region containing peptide. Suitable amphiphilic-region containing peptides include: melittin, the magainins, fusion segments from H. influenza hemagglutinin (HA) protein, HIV segment I from the cytoplasmic tail of HIV1 gp41, and amphiphilic segments from viral env membrane proteins including those from avian leukosis virus (ALV), bovine leukemia virus (BLV), equine infectious anemia (EIA), feline immunodeficiency virus (FIV), hepatitis virus, herpes simplex virus (HSV) glycoprotein H, human respiratory syncytia virus (hRSV), Mason-Pfizer monkey virus (MPMV), Rous sarcoma virus (RSV), parainfluenza virus (PINF), spleen necrosis virus (SNV), and vesicular stomatitis virus (VSV). Other suitable peptides include microbial and reptilian cytotoxic peptides. The specific peptides or other molecules having greatest utility can be identified using four kinds of assays: 1) ability to disrupt and induce leakage of aqueous markers from liposomes composed of cell membrane lipids or fragments of cell membranes, 2) ability to induce fusion of liposomes composed of cell membrane lipids or fragments of cell membranes, 3) ability to induce cytoplasmic release of particles added to cells in tissue culture, and 4) ability to enhance plasmid expression by particles in vivo tissues when administered locally or systemically.

The fusogenic moiety also may be comprised of a polymer, including peptides and synthetic polymers. In one embodiment, the peptide polymer comprises synthetic peptides containing amphipathic aminoacid sequences such as the “GALA” and “KALA” peptides (Wyman T B, Nicol F, Zelphati O, Scaria P V, Plank C, Szoka F C Jr, Biochemistry 1997, 36:3008-3017; Subbarao N K, Parente R A, Szoka F C Jr, Nadasdi L, Pongracz K, Biochemistry 1987 26:2964-2972 or Wyman supra, Subbarao supra). Other peptides include non-natural aminoacids, including D aminoacids and chemical analogues such as peptoids, imidazole-containing polymers. Suitable polymers include molecules containing amino or imidazole moieties with intermittent carboxylic acid functionalities such as ones that form “salt-bridges,” either internally or externally, including forms where the bridging is pH sensitive. Other polymers can be used including ones having disulfide bridges either internally or between polymers such that the disulfide bridges block fusogenicity and then bridges are cleaved within the tissue or intracellular compartment so that the fusogenic properties are expressed at those desired sites. For example a polymer that forms weak electrostatic interactions with a positively charged fusogenic polymer that neutralizes the positive charge could be held in place with disulfide bridges between the two molecules and these disulfides cleaved within an endosome so that the two molecules dissociate releasing the positive charge and fusogenic activity. Another form of this type of fusogenic agent has the two properties localized onto different segments of the same molecule and thus the bridge is intramolecular so that its dissociation results in a structural change in the molecule. Yet another form of this type of fusogenic agent has a pH sensitive bridge.

Other polymers can be used including polymers with amino or imidazole moieties with intermittent carboxylic acid functionalities such as ones that form “salt-bridges” either internally or externally including forms that the bridging is pH sensitive. In one embodiment, the polymer has a chemical structure as shown below.

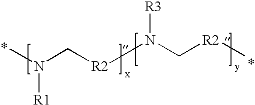

where R1 is a hydrocarbon or a hydrocarbon substituted with an amine, guanidinium, or imidazole moiety, R2 is a lower alkyl group as defined above, and R3 is a hydrocarbon or a hydrocarbon substituted with a carboxyl, hydroxyl, sulfate, or phosphate moiety. In one embodiment the polymer is designed to bear an excess positive charge such as when R1 contains an amine or guanidinium and R3 contains a carboxyl with X about equal with Y or greater than Y or when R1 contains an imidazole and R3 contains a carboxyl with X in excess of Y. In another embodiment the polymer is designed to bear an excess negative charge so typically Y is in excess of X. In yet another embodiment the polymer is designed to have a net charge near neutrality and the X to Y ratio is adjusted accordingly.

In another embodiment, the polymer has a chemical structure as described below.

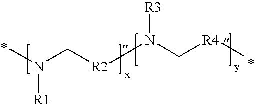

where R1 is a hydrocarbon or a hydrocarbon substituted with an amine, guanidinium, or imidazole moiety, R2 and R4 independently are lower alkyl groups as defined above, and R3 is a hydrocarbon or a hydrocarbon substituted with a carboxyl, hydroxyl, sulfate, or phosphate moiety. In one embodiment the polymer is designed to bear an excess positive charge such as when R1 contains an amine or guanidinium and R3 contains a carboxyl with X about equal with Y or greater than Y or when R1 contains an imidazole and R3 contains a carboxyl with X in excess of Y. In another embodiment the polymer is designed to bear an excess negative charge so typically Y is in excess of X. In yet another embodiment the polymer is designed to have a net charge near neutrality and the X to Y ratio is adjusted accordingly.

The fusogenic moiety also may comprise a membrane surfactant polymer-lipid conjugate. Suitable conjugates include Thesit™, Brij 58™, Brij 78™, Tween 80™, Tween 20™, C₁₂E₈, C₁₄E₈, C₁₆E₈ (CₙFₙ=hydrocarbon poly(ethylene glycol) ether where C represents hydrocarbon of carbon length N and E represents poly(ethylene glycol) of degree of polymerization N), Chol-PEG 900, analogues containing polyoxazoline or other hydrophilic polymers substituted for the PEG, and analogues having fluorocarbons substituted for the hydrocarbon. Advantageously, the polymer will be either biodegradable or of sufficiently small molecular weight that it can be excreted without metabolism. The skilled artisan will recognize that other fusogenic moieties also may be used without departing from the spirit of the invention.

Assembly of the Core Complex

The core complex advantageously will be self-assembling when mixing of the components occurs under appropriate conditions. Suitable conditions for preparing the core complex generally permit the charged component that is present in charge molar excess at the end of the mixing to be in excess throughout the mixing. For example, if the final preparation is a net negative charge excess then the cationic agent is mixed into the anionic agent so that the complexes formed never have a net excess of cationic agent. Another suitable condition for preparing the core complex utilizes a continuous mixing process including mixing of the core components in a static mixer. A static mixer produces turbulent flow and preferably low shear force mixing in two or more fluid streams flowing into and through a stationary device resulting in a mixed fluid that exits the device. For core complexes low shear force mixing is expecially important when the nucleic acid is fragile to shear. Specifically, aqueous solutions of nucleic acid and core complex-forming moieties (such as a cationic lipid) are fed together into a static mixer (available from, for example, American Scientific Instruments, Richmond, Calif.), where the streams are split into inner and outer helical streams that intersect at several different points causing turbulence and thereby promoting mixing. The use of commercially available static mixers ensures that the results obtained are operator-independent, and are scalable, reproducible, and controllable. The core complex particles so produced are homogeneous, stable, and can be sterile filtered. When the core complex is intended to contain a nuclear targeting moiety and/or a fusogenic moiety, these components may be added directly into the streams entering the static mixer so that they are automatically incorporated into the core complex as it is formed.

The component streams intersect in the mixer, whereby shearing and mixing of the DNA and polymer are induced, whereby particles of a complex of DNA and polymer are formed. The resulting preparations may be tested for mean particle size in nanometers and distribution through dynamic light scattering using, for example, a Coulter N4 Plus Submicron Particle Sizer (Coulter Corporation, Miami, Fla.). Mean particle sizes and standard deviaitons can be determined by the unimodal and Size Distribution Processing (SDP), or “intensity” methods.

In the above methods, a laser is directed through a preparation of the particles. Dynamic light scattering is measured as a result of the Brownian motion of the particles. The dynamic light scattering-which is measured then is correlated-to particle size. In the unimodal method, the size distribution is determined by placing the sizes of the particles on a Gaussian curve. In the SDP method, size distribution is determined by a FORTRAN program called CONTIN. Such methods also are described further in the Coulter N4 Plus Submicron Particle Sizer Reference Manual (November 1995).

When the fusogenic moiety is not incorporated directly into the core moiety, it typically is present as a shell surrounding or enveloping the core complex. In this situation the fusogenic shell is anchored to the core complex either electrostatically, covalently, or via hydrophobic interaction, or by a combination of such forces. When the fusogenic moiety is electrostatically anchored it interacts with charged groups of either the nucleic acid, or the complex forming agent, or both, through charge-charge interactions. Presence of multivalent electrostatic interactions allows binding stability but also accomodates appropriate release within the target tissue and cell. One specific form is a fusogenic peptide sequence coupled to a cationic peptide sequence where the cationic sequence insures that the peptide either incorporates into the core complex at the time of its formation or it incorporates onto the surface of a negatively charged core complex after its formation. One example of this type of moiety and its incorporation is the inclusion of a peptide comprised of a linear sequence of 14 lysine residues coupled to a short hydrophobic amino acid sequence from the fusion domain of H. influenze HA protein shown in Example 46. Other examples include use of synthetic cationic polymers such as PEI coupled with fusogenic segment polymers such as poly[2-(diethylamino)ethyl methacrylate] (PDEAMA) or N-isopropylacrylamide methacrylic acid copolymers.

When the fusogenic moiety is anchored with hydrophobic interactions it contains a segment or moiety that associates with the core complex in such a manner that the association reduces contact with the aqueous solution and thereby reduces the energy of the anchored complex. In one embodiment, the anchor hydrophobic interactions are between hydrocarbon moieties of the fusogenic moiety and hydrocarbon moieties of the core complex. One specific form utilizing hydrophobic anchoring are diacyl lipids conjugated with a fusogenic moiety where the lipid portion interacts strongly with core complexes formed with cationic lipids. In another embodiment, the anchor hydrophobic interactions are between fluorocarbon moieties of the fusogenic moiety and fluorocarbon moieties of the core complex. Other forms of hydrophobic interaction forces that enable suitable anchoring are possible.

When the fusogenic moiety is covalently linked to the core complex, covalent coupling occurs: (1) to complex forming reagents; (2) to a compound that becomes incorporated in the complex at its time of formation; (3) to the surface of a preformed complex; or (4) to a compound that associates with the surface of a preformed complex. In one embodiment the linkage preferably is cleaved upon entry of the vector into a target tissue or cell. This cleavage may be achieved by anchoring the fusogenic layer via a cleavable linkage. Examples include: (1) an acid labile linkage, such as a Schiff's base or a hydrazone or vinyl ether; (2) a reducible linkage such as a disulfide linkage; or (3) one of the linkers described below for use in attachment of the outer steric layer. Acid labile linkers are cleaved in the acid conditions that prevail in targeted tissues or in intracellular compartment such as the endosome structure into which the vector first will be transported upon cellular uptake by most mechanisms. In one embodiment, the fusogenic layer has a hydrophobic nature such that it forms a layer in which water is largely excluded. When such a layer is formed on the core complex, it can be generated by numerous possible methods such as addition along with the complex forming agent where the layer forms by self assembly or by addition in a second step once the core complex has been formed. In one embodiment, the layer is formed at the same time as the core complex as illustrated in Examples 38-43. In another embodiment, the layer is formed by a second mixing step where a core complex is formed in the first mixing step and then the layer is added by a subsequent mixing step between the core complex and the reagent that forms the layer on the pre-existing complex.

In one embodiment of the invention, the use of core complexes which are negative or neutral in surface charge is preferred. In this embodiment, the outer shell conveys target tissue and cell binding and uptake properties in contrast to the cationic complex-anionic cell electrostatic binding mechanism that is thought to provide binding and uptake by positively-charged core complexes. By allowing use of neutral or negative surface charge core complexes, numerous benefits can be realized. The reduction or elimination of electrostatic interactions with positive surface charge vector colloids can reduce or eliminate non-specific interactions leading to phagocytic clearance, to toxicity in non-target tissues and organs, and to cell toxicity in target tissues and organs.

It is to be understood that the present invention is not to be limited to the treatment of any particular disease or disorder.

The particles which include a nucleic acid sequence encoding a therapeutic agent, may be administered to an animal in vivo as part of an animal model for the study of the effectiveness of a gene therapy treatment. The particles may be administered in varying doses to different animals of the same species, whereby the particles will transfect cells in the animal. The animals then are evaluated for the expression of the desired therapeutic agent in vivo in the animal. From the data obtained from such evaluations, one may determine the amount of particles to be administered to a human patient.

In another embodiment, the particles may be employed to transfect cells in vitro. The cells, which now include a nucleic acid sequence encoding a therapeutic agent, may be administered to a host such as hereinabove described, in order to express the therapeutic agent and/or provide a therapeutic effect in the host. Cells which may be transfected and methods of administration may be selected from those hereinabove described.

The particles of the present invention also may be employed to transfect cells of an organ in vitro. The organ, which now includes cells which include a nucleic acid sequence encoding a therapeutic agent, may be transplanted into an animal, whereby the transplanted organ expresses the therapeutic agent in the animal and/or provide a therapeutic effect in the animal. The animal may be a mammal, including human and non-human primates.

The particles of the present invention also may be employed in the in vitro transfection of cells, which are contained in a cell culture containing a mixture of cells. Upon transduction of the cells in vitro, the cells produce the therapeutic agent or protein in vitro. The therapeutic agent or protein then may be obtained from the cell culture by means known to those skilled in the art.

The particles also may be employed for the transfection of cells in vitro in order to study the mechanism of the genetic engineering of cells in vitro.

Outer Shell Moiety

It is known that polyethylene glycol (PEG), an uncharged hydrophilic polymer, can provide a steric barrier for oligonucleotide/cationic lipid complexes (Meyer et al., J. Biol. Chem. 273:15621 (1998); Scaria supra, Philips supra). The present invention improves upon conventional uses of steric barriers by providing a barrier that is anchored to the core complex. The barrier also may optionally contain targeting moieties that enhance binding of the vectors to the target tissue and cell and also that may optionally be anchored via an attachment that is cleaved at target tissues or in intracellular compartments into which the vector typically first will be transported upon cellular uptake.

In embodiments where the core complex is anchored to a fusogenic shell moiety, the outer steric layer is in turn anchored, as described below, to the core complex, the fusogenic shell, or to both. In embodiments where the fusogenic moiety is incorporated directly into the core complex, the steric layer is anchored directly to the core complex.

The outer steric layer preferably comprises a hydrophilic, biodegradable polymer. If the polymer is not biodegradable then a relatively low molecular weight (<30 kDaltons) polymer is used. The polymer may also exhibit solubility in both polar and non-polar solvents. Suitable polymers include PEG (of various molecular weights), polyvinylpyrrolidone (PVP), and polyvinylalcohol, polyvinylmethylether, polyhydroxypropyl methacrylate, polyhydroxypropylmethacrylamide, polyhydroxyethyl acrylate, polymethacrylamide, polydimethylacrylamide, polylactic acid, polyglycolic acid, polymethyloxazoline, polyethyloxazoline, polyhydroxyethyloxazoline, polyhydroxypropyloxazoline, or polyaspartamide which are well known in the art (U.S. Pat. No. 5,631,018).

Other suitable polymers include those that will form a steric barrier on colloidal particulates of at least 5 nm “thickness” or greater as determined by reduction in zeta potential (Woodle et al., Biophys. J. 61:902 (1992)) or other such assays. Further suitable polymers include those that contain branches. In one embodiment, the hydroxyl functions of a glucose moiety are used to conjugate multiple steric polymers, one of which is anchored to the core complex. In another embodiment, the amine functions of a lysine are used to conjugate two steric polymers and the carboxyl function is used with a steric polymer linker to conjugate onto the core complex.

When PEG is used as the hydrophilic polymer conjugate, the PEG preferably has a molecular weight of between about 1,000 to about 50,000 daltons. Typically, the PEG chain has a molecular weight of about 2,000 to about 20,000 daltons. Mixtures of molecular weight can also be used which can have particular advantages for combining steric properties best found in a large polymer, e.g. blocking cellular interactions, with those best found in a small polymer, e.g. blocking small protein interactions. When used without a ligand at the end distal to coupling, the PEG contains an unreactive methoxy group at its free end, and is coupled to the linking segment through a reactive chemical group. Methods of preparing such linking is well known in the art as summarized in a recent text book on conjugation (Greg T. Hermanson, Biconjugate techniques, Academic Press Inc., San Diego, 1996).

Alternative polymers include, but are not limited to, polylactic acid, polyglycolic acid, polyvinylpyrrolidone, polymethacrylamide, polyethyloxazoline, polymethyloxazoline, polydimethylacrylamide, polyvinylmethylether, polyhydroxypropyl methacrylate, polyhydroxypropylmethacrylamide, polyhydroxyethyl acrylate, polyhydroxyethyloxazoline, polyhydroxypropyloxazoline, or polyaspartamide. As described above for PEG, when used without a ligand at the end distal to coupling, each of these hydrophilic polymers preferably has an unreactive group or a hydroxyl at its free end, and is coupled to the linking segment through a reactive chemical group.

Anchoring is provided either by electrostatic, covalent, or hydrophobic interaction, or by a combination of such forces. When the outer shell is electrostatically anchored it interacts with charged groups located on the nucleic acid or on the complex forming agent, or both through charge-charge interations. The presence of multivalent electrostatic interactions not allows binding stability but also accomodates appropriate release within the target tissue and cell. When the outer shell is anchored with hydrophobic interactions it contains a segment or moiety that associates with the core complex in such a manner that the association reduces contact with the aqueous solution and thereby reduces the energy of the anchored complex. In one embodiment, the anchor hydrophobic interactions are between hydrocarbon moieties of the outer shell and hydrocarbon moieties of the core complex. In another embodiment, the anchor hydrophobic interactions are between fluorocarbon moieties of the outer shell and fluorocarbon moieties of the core complex. Other forms of hydrophobic interaction forces that enable suitable anchoring are possible. In one embodiment, such hydrophobic achors are comprised of peptide sequences that associate and intercalate with lipid bilayers such as membrane anchor domains including sequences from membrane proteins such as cytochrome b5 (Thr-Asn-Trp-Val-Ile-Pro-Ala-Ile-Ser-Ala-Val-Val-Val-Ala-Leu-Met-Tyr-Arg-Ile-Tyr-Thr-Ala) (SEQ ID NO:1) or membrane spanning sequences.

When the outer shell is covalently linked to the core complex, covalent coupling is provided to complex forming reagents, or alternatively through covalent coupling to a compound that becomes incorporated in the complex at its time of formation, or alternatively through covalent coupling to the surface of a preformed complex, or alternatively through covalent coupling to a compound that associates with the surface of a preformed complex. In one embodiment the linkage preferably is cleaved upon entry of the vector into a target tissue or cell. This cleavage may be achieved by anchoring the outer shell via cleavable linkage such as an acid labile linkage, such as a Schiff's base or a hydrazone, vinyl ether, or as a reducible linkage such as a disulfide linkage, or one of the linkers described below for use in attachment of the outer steric layer. Acid labile linkers are cleaved in the acid conditions that prevail in targeted tissues or in intracellular compartment such as the endosome structure into which the vector first will be transported upon cellular uptake by most mechanisms. In one embodiment, the fusogenic layer has a hydrophobic nature such that it forms a layer in which water is largely excluded. When such a layer is formed on the core complex, it can be generated by numerous possible methods such as addition along with the complex forming agent and the layer forms by self assembly or by addition in a second step once the core complex has been formed.

In another embodiment, the polymer is used with a ligand. The ligand is comprised of a molecule that provides for binding to target tissues and cells such that the nucleic acid payload exerts its biological activity. Suitable ligands include proteins, peptides, and their chemical analogues, carbohydrates, and small molecules. In one embodiment, the ligand is attached to the core complex in a manner similar to that of the fusogenic moiety or of the steric polymer. In another embodiment, the ligand is attached to the steric polymer at the end distal to its coupling to the core complex. Suitable attachment of the ligand include stable covalent linkage, cleavable linkage, and non-covalent attachment that retains the ligand until the desired binding event can occur.

The Targeting Moiety

To enhance binding of the vector to target tissue or cells, the outer shell layer advantageously will include at least one targeting moiety that permits highly specific interaction of the vector with the target tissue or cell. More specifically, in one embodiment, the vector preferably will include an unshielded ligand attached to the outer layer, effective for ligand-specific binding to a receptor molecule on a target tissue and cell surface (Woodle et al., Small molecule ligands for targeting long circulating liposomes, in Long Circulating Liposomes: Old drugs, new therapeutics, Woodle and Storm eds., Springer, 1998, p 287-295). In another embodiment, the vector preferably will include a shielded ligand attached within the outer layer or at the surface of the core complex where the outer layer is lost under defined tissue or target conditions, revealing the ligand so that it can bind to the target tissue or cell. The vector may include two or more targeting moieties, depending on the cell type that is to be targeted. Use of multiple (two or more) targeting moieties can provide additional selectivity in cell targeting, and also can contribute to higher affinity and/or avidity of binding of the vector to the target cell. When more than one targeting moiety is present on the vector, the relative molar ratio of the targeting moieties may be varied to provide optimal targeting efficiency. Methods for optimizing cell binding and selectivity in this fashion are known in the art. The skilled artisan also will recognize that assays for measuring cell selectivity and affinity and efficiency of binding are known in the art and can be used to optimize the nature and quantity of the targeting ligand(s).

Suitable ligands include, but are not limited to: vascular endothelial cell growth factor for targeting endothelial cells: FGF2 for targeting vascular lesions and tumors; somatostatin peptides for targeting tumors; transferrin for targeting tumors; melanotropin (alpha MSH) peptides for tumor targeting; ApoE and peptides for LDL receptor targeting; von Willebrand's Factor and peptides for targeting exposed collagend; Adenoviral fiber protein and peptides for targeting Coxsackie-adenoviral receptor (CAR) expressing cells; PD 1 and peptides for targeting Neuropilin 1; EGF and peptides for targeting EGF receptor expressing cells; and RGD peptides for targeting integrin expressing cells.

Other examples include (i) folate, where the composition is intended for treating tumor cells having cell-surface folate receptors, (ii) pyridoxyl, where the composition is intended for treating virus-infected CD4+ lymphocytes, or (iii) sialyl-Lewis^{o}, where the composition is intended for treating a region of inflammation. Other peptide ligands may be identified using methods such as phage display (F. Bartoli et al., Isolation of peptide ligands for tissue-specific cell surface receptors, in Vector Targeting Strategies for Therapeutic Gene Delivery (Abstracts form Cold Spring Harbor Laboratory 1999 meeting), 1999, p4) and microbial display (Georgiou et al., Ultra-High Affinity Antibodies from Libraries Displayed on the Surface of Microorganisms and Screened by FACS, in Vector Targeting Strategies for Therapeutic Gene Delivery (Abstracts form Cold Spring Harbor Laboratory 1999 meeting), 1999, p 3.). Ligands identified in this manner are suitable for use in the present invention.

In a particular embodiment, the targeting ligand may be somatostatin or a somatostatin analog. Somatostatin has the sequence AGCLNFFWKTFTSC (SEQ ID NO:2), and contains a disulfide bridge between the cysteine residues. Many somatostatin analogs that bind to the somatostatin receptor are known in the art and are suitable for use in the present invention. See for example, U.S. Pat. No. 5,776,894, which is incorporated herein by reference in its entirety. Particular somatostatin analogs that are useful in the present invention are analogs having the general structure F*CY-(DW)KTCT, where DW is D-tryptophan and F* indicates that the phenylalanine residue may have either the D- or L-absolute configuration. As in somatostatin itself, these compounds are cyclic due to a disulfide bond between the cysteine residues. Advantageously, these analogs may be derivatized at the free amino group of the phenylalanine residue, for example with a polycationic moiety such as a chain of lysine residues. The skilled artisan will recognize that other somatostatin analogs that are known in the art may advantageously be used in the invention.

Furthermore, methods have been developed to create novel peptide sequences that elicit strong and selective binding for target tissues and cells such as “DNA Shuffling” (W. P. C. Stremmer, Directed Evolution of Enzymes and Pathways by DNA Shuffling, in Vector Targeting Strategies for Therapeutic Gene Delivery (Abstracts form Cold Spring Harbor Laboratory 1999 meeting), 1999, p.5.) and these novel sequence peptides are suitable ligands for the invention. Other chemical forms for ligands are suitable for the invention such as natural carbohydrates which exist in numerous forms and are a commonly used ligand by cells (Kraling et al., Am. J. Path. 150:1307 (1997) as well as novel chemical species, some of which may be analogues of natural ligands such as D-amino acids and peptidomimetics and others which are identifed through medicinal chemistry techniques such as combinatorial chemistry (P. D. Kassner et al., Ligand Identification via Expression (LIVEθ): Direct selection of Targeting Ligands from Combinatorial Libraries, in Vector Targeting Strategies for Therapeutic Gene Delivery (Abstracts form Cold Spring Harbor Laboratory 1999 meeting), 1999, p8.).

The targeting layer is composed of ligands that provide the desired tissue and cell specific binding exposed at the surface of the complex, either that of the core complex, the surface of the fusogenic layer, or the surface of the protective, steric, layer. The ligands are covalently attached to the colloid such that their exposure is adequate for tissue and cell binding. Anchoring is provided by covalent coupling to complex forming reagents, or alternatively through covalent coupling to a compound that becomes incorporated in the complex at its time of formation, or alternatively through covalent coupling to the surface of a preformed complex, or alternatively through covalent coupling to a compound that associates with the surface of a preformed complex.

For example a peptide ligand can be covalently coupled to a steric polymer such as polyoxazoline which is covalently coupled at its distal end to a polycation such as linear PEI. The PEI will form a layered colloid complex with the nucleic acid payload forming a surface shell of steric polymer with peptide ligands exposed on the surface. Alternatively, this same peptide conjugate can be combined with a polycation such as linear PEI or a cationic lipid in an aqueous solution that is then used to condense a nucleic acid payload into a layered colloid with the ligand exposed above a surface steric polymer shell.

Alternatively this same peptide conjugate can be complexed with a negatively charged complex of nucleic acid payload at least partially condensed with a polycation or cationic lipid resulting in a layered colloid with the ligand exposed above a surface steric polymer shell. Similarly, a peptide ligand can be covalently coupled to a steric polymer such as polyoxazoline which is covalently coupled at its distal end with a lipid and this conjugate used as above with polycations and/or cationic lipids and/or neutral or negative lipid colloids containing a nucleic acid payload.

The number of targeting molecules present on the outer layer will vary, depending on factors such as the avidity of the ligand-receptor interaction, the relative abundance of the receptor on the target tissue and cell surface, and the relative abundance of the target tissue and cell. Nevertheless, 25-100 targeting molecules on the surface of each vector usually provides suitable enhancement of cell targeting.

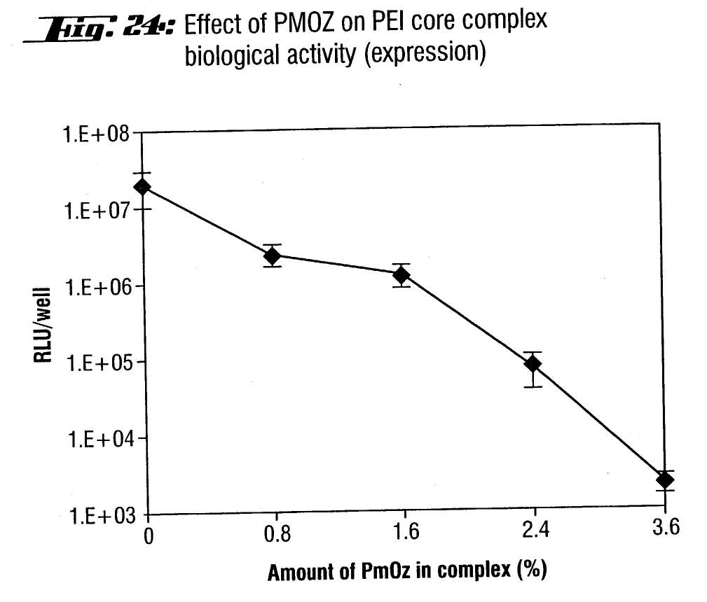

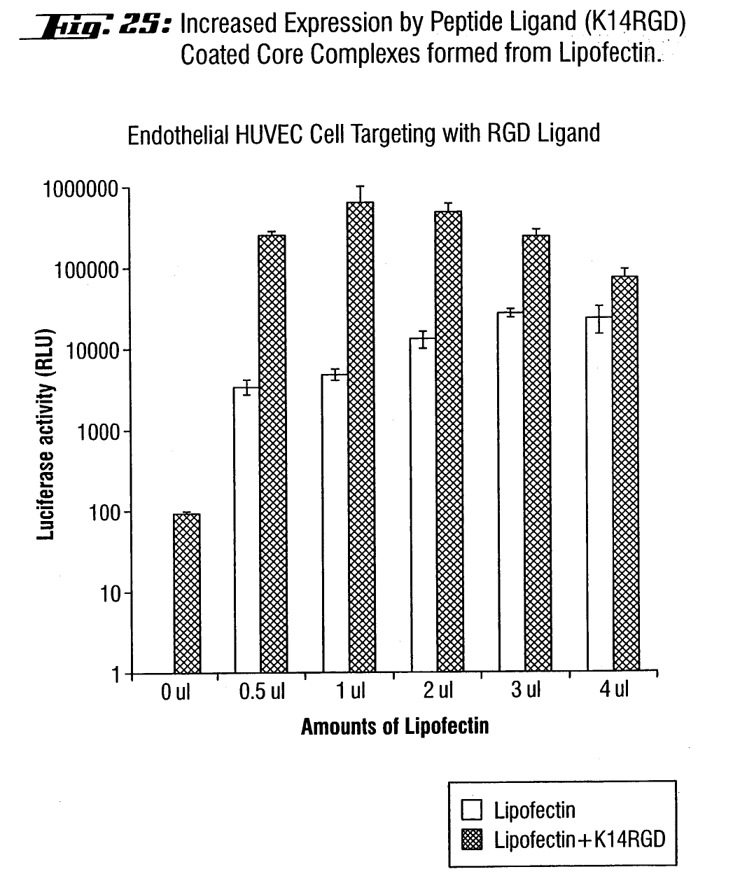

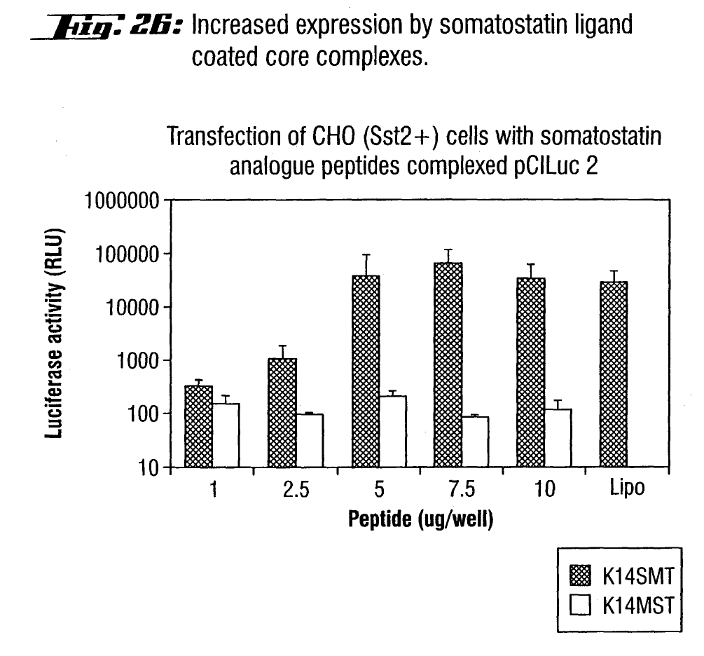

The presence of the targeting moiety leads to the desired enhancement of binding to target tissue and cells. An appropriate assay for such binding may be ELISA plate assays, cell culture expression assays, or any other binding assays. One example of binding is shown in Example 48 and FIGS. 25 and 26.

Anchoring of the Outer Shell Moiety

As described above, the outer steric layer of the outer shell moiety is anchored to the inner fusogenic layer, to the core complex, or both. This anchoring may be either electrostatically, covalently, or with hydrophobic interaction, or a combination of such forces. When the outer shell is electrostatically anchored it interacts with charged groups of either the nucleic acid, or the complex forming agent, or both, through charge-charge interactions. Presence of multivalent electrostatic interactions allows binding stability but also accomodates appropriate release within the target tissue and cell. When the outer shell is anchored with hydrophobic interactions it contains a segment or moiety that associates with the core complex in such a manner that the association reduces contact with the aqueous solution and thereby reduces the energy of the anchored complex.

In one embodiment, such achors are comprised of peptide sequences that associate and intercalate with lipid bilayers such as membrane anchor domains including sequences from membrane proteins such as cytochrome b5 (Thr-Asn-Trp-Val-Ile-Pro-Ala-Ile-Ser-Ala-Val-Val-Val-Ala-Leu-Met-Tyr-Arg-Ile-Tyr-Thr-Ala) (SEQ ID NO: 1) or membrane spanning sequences. In one embodiment, the anchor hydrophobic interactions are between hydrocarbon moieties of the outer shell and hydrocarbon moieties of the core complex. In another embodiment, the anchor hydrophobic interactions are between fluorocarbon moieties of the outer shell and fluorocarbon moieties of the core complex. Other forms of hydrophobic interaction forces that enable suitable anchoring are possible.

When the outer shell is covalently linked to the core complex, covalent coupling occurs: (1) to complex forming reagents; (2) to a compound that becomes incorporated in the complex at its time of formation; (3) to the surface of a preformed complex; or (4) to a compound that associates with the surface of a preformed complex.

When the outer shell is anchored to the fusogenic layer via a covalent bond, the linkage may be stable, and in this embodiment, the outer layer will be shed along with the fusogenic layer upon cell entry. One example of a stable linkage is a carbamate linkage. In another embodiment, the linkage preferably is cleaved upon entry of the vector into a target tissue or cell. In one embodiment, the fusogenic layer has a hydrophobic nature such that it forms a layer in which water is largely excluded. When such a layer is formed on the core complex, it can be generated by numerous possible methods such as addition along with the complex forming agent where the layer forms by self assembly or by addition in a second step once the core complex has been formed.

When the outer layer is anchored directly to the core complex, it preferably is cleavable under the conditions prevailing in the endosome. This cleavage may be achieved by anchoring the outer shell via cleavable linkage such as an acid labile linkage or as a reducible linkage such as a disulfide linkage. Acid labile linkers are cleaved in the acid conditions that prevail in targeted tissues or in intracellular compartment such as the endosome structure into which the vector typically is first transported upon cellular uptake. Suitable cleavable linkages include a disulfide bond, and an acid labile linkage such as a Schiff's base, or a hydrazone, or a vinyl ether. For example, the core complex may contain free amine groups, and the steric layer may contain pendent aldehyde groups. Mixing of the core complex with the steric layer component will result in formation of a Schiff's base between the core complex and the steric layer. Alternatively, for example, a disulfide bond can be formed between free sulfhydryl groups present on the core complex and the steric layer, respectively. In a preferred embodiment, the cleavable linkage layer comprises a pH sensitive covalent bond. More preferably, the pH-sensitive covalent bond is selected from the group consisting of:

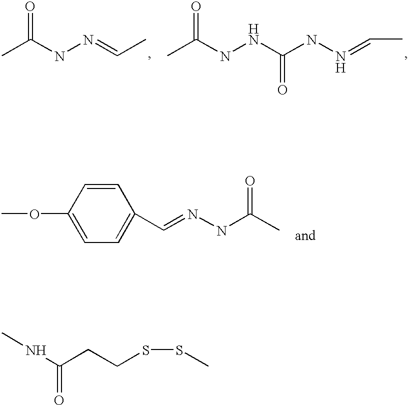

Method of Administration of the Vectors

The vectors are administered parenterally through systemic and local injection routes and they also may be administered ex-vivo.

In vitro Testing of the Vectors

Methods of in vitro testing of the vectors of the invention are well known in the art. For example, they can be tested for the ability to provide delivery to cells and tissues in culture as described in Examples 40 and 42 or they can be tested for colloidal and physicochemical properties as described in Examples 35 and 44.

In vivo Efficacy Testing of the Vectors

Methods of measuring the in vitro efficacy of the vectors of the invention are well known in the art. For example, when the vectors are used for the treatment of a disease in a mammal, efficacy of the vector can be determined by study of the amelioration of one or more symptoms of the disease. Advantageously, the in vivo efficacy can use measurement of defined clinical end points that are characteristic of the progress or extent of a disease.

These and other features and advantages of the invention will be more fully appreciated with the following examples, which are provided for illustrative purposes only, and are not intended to be limiting of the scope of the invention.

The following examples illustrate the present invention; the temperatures are given in degrees Celsius. The following abbreviations are used:

BOC=tert.-butyloxycarbonyl;

THF=tetrahydrofuran;

hexane=n-hexane;

ether=diethyl ether.

Concerning nomenclature: when numbering the different nitrogen atoms, the terminal amino nitrogens are treated as substituents of the terminal carbon atoms, while the non-terminal nitrogen atoms are interpreted as aza substitutions of CH₂ groups and are numbered accordingly. Therefore e.g. the 4 nitrogen atoms in spermine are designated N¹, N⁴, N⁹ and N¹²:

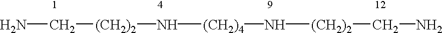

(1,12-diamino-4,9-diazadodecane)

### EXAMPLE 1

### N⁴-[(2-hydroxy)-n-tetradecyl]-spermidine Trihydrochloride

A solution of 6 g (0.1646 moles) of hydrogen chloride in 50 ml of ethyl acetate was added whilst stirring, at room temperature, to a solution of 8.8 g (0.0158 moles) of N¹,N⁸-di-BOC-N⁴-[(2-hydroxy)-n-tetradecyl]-spermidine in 50 ml of ethyl acetate. After stirring for 1.25 hours, the crystals that had precipitated from the reaction mixture were filtered. The hygroscopic crude product was dissolved in water and chromatographed on a column charged with Amberlite XAD 1180 adsorber resin (in water), whereby elution took place first of all with water and then with a mixture of water and isopropanol (9:1 or 3:1). The fractions containing the product were combined, concentrated in a water jet vacuum, and lyophilized under a high vacuum. The title compound was obtained as a lyophilizate with a water content of 4.25%, R_{f}: 0.25 [thin-layer chromatography plates silica gel 60 F₂₅₄; solvent: methylene chloride/methanol/30% aqueous ammonia solution (10:3.5:1)].

The starting compounds were produced as follows:

a) N¹,N⁸-di-BOC-N⁴-[(2-hydroxy)-n-tetradecyl]-spermidine

12.49 g (0.05 moles) of 1,2-tetradecene oxide (85%) were added to a solution of 17.27 g (0.05 moles) of N¹,N⁸-di-BOC-spermidine in 200 ml of ethanol. The reaction mixture was heated for 2 hours under reflux and then a further 3.44 g (0.01377 moles) of 1,2-tetradecene oxide were added. After heating for 16.5 hours under reflux, the reaction mixture was concentrated by evaporation. Purification of the oily crude product was effected by flash chromatography on silica gel of grain size 0.04-0.063 mm. The product-containing fractions which have been eluted with a methylene chloride/methanol mixture (19:1) were combined and concentrated by evaporation under vacuum. The title compound was obtained in the form of an oil, R_{f}: 0.80

[solvent: methylene chloride/methanol/30% aqueous ammonia solution (40:10:1)].

b) N¹,N⁸-di-BOC-spermidine

A solution of 221.67 g (0.90 moles) of 2-(BOC-oxyimino)-2-phenylacetonitrile in 630 ml of THF was added dropwise at 0-5° whilst stirring, under a nitrogen atmosphere, over the course of 2 hours, to a solution of 65.34 g (0.45 moles) of spermidine in 630 ml of THF. The reaction mixture was stirred for 16 hours at room temperature, then concentrated by evaporation under vacuum, and the oily residue was partitioned between ether and diluted hydrochloric acid (pH 3). The phase containing hydrochloric acid was rendered basic with 30% sodium hydroxide solution (pH 10), the desired product was extracted with ether, the ether extract washed with saturated sodium chloride solution, the organic phase dried over sodium sulfate and concentrated by evaporation under vacuum. After recrystallization of the residue from ether-hexane, the title compound was obtained, m.p. 85-86°. By concentrating the mother liquor, a second batch of the title compound was obtained, m.p. 78-82°.

### EXAMPLE 2

### N⁴-[(2-hydroxy)-n-tetradecyl]-spermidine Trioxalate

A solution of 10.17 g (0.08067 moles) of oxalic acid dihydrate in 90 ml of water was added whilst stirring to a solution of 15 g (0.02689 moles) of N¹,N⁸-di-BOC-N⁴-[(2-hydroxy)-n-tetradecyl)]-spermidine (example 1a) in 30 ml of ethanol. The reaction mixture was stirred for 5 hours at 90° and subsequently concentrated under vacuum. After cooling to 0°, the title compound precipitated in crystalline form from the concentrate which had been mixed with ethanol, m.p. 180° (decomp.).

### EXAMPLE 3

### N⁵-[(2-hydroxy)-n-decyl]-homospermidine Trihydrochloride

A solution of 1.276 g (0.035 moles) of hydrogen chloride in 10 ml of ethyl acetate was added whilst stirring, at room temperature, to a solution of 2.89 g (0.0056 moles) of N¹,N⁹-di-BOC-N⁵-[(2-hydroxy)-n-decyl]-homospermidine in 10 ml of ethyl acetate. Stirring was effected for 20 minutes at room temperature and for 20 minutes at 0°. The precipitated product was filtered, washed with cold ethyl acetate, dissolved in water and chromatographed with water on a column charged with Amberlite XAD 1180 adsorber resin. After lyophilization of the combined product-containing fractions, the title compound was obtained with a water content of 4.5%, R_{f}: 0.28 (solvent as for example 1).

The starting compounds were produced as follows:

a) N¹,N⁹-di-BOC-N⁵-[(2-hydroxy)-n-decyl]-homospermidine

2.63 g (0.0168 moles) of 1,2-decene oxide were added to a solution of 5.03 g (0.014 moles) of N¹,N⁹-di-BOC-homospermidine in 50 ml of ethanol. The reaction mixture was boiled under reflux for 22 hours, then a further 0.52 g (0.00333 moles) of 1,2-decene oxide were added, heating continued for 18 hours under reflux and then the mixture was concentrated by evaporation under vacuum. Purification of the oily crude product was effected by flash chromatography on silica gel. Elution was carried out with methylene chloride and methylene chloride/methanol mixtures with a methanol content of 1%, or 2.5%, or 5%, or 10%. The title compound was obtained in the form of an oil,

R_{f}: 0.39 [solvent: methylene chloride/methanol (9:1)].

b) N¹,N⁹-di-BOC-homospermidine

17 g of palladium on activated carbon (10% Pd) were added to a solution of 167.7 g (0.373 moles) of N⁵-benzyl-N¹,N⁹-di-BOC-homospermidine (Bergerone et al., Synthesis 1982:689) in a mixture of 1200 ml of methanol and 31.9 ml of conc. hydrochloric acid, and hydrogenation was carried out at 30° until the hydrogen uptake has ended. After filtration and evaporation of the filtrate to dryness, the crystalline residue (hydrochloride of the title compound) was dissolved in 2 litres of water and the aqueous solution (pH 4) was adjusted to pH 3 by adding 4N hydrochloric acid. The product was washed with ether, the aqueous phase adjusted to pH 10 by adding 30% sodium hydroxide solution, and the oiled product was extracted with three portions of ether, each of 500 ml. After washing the combined ether phases with conc. aqueous sodium chloride solution, drying over sodium sulfate and evaporating under vacuum, the title compound was obtained in the form of an oil which gradually crystallized, m.p. 42-46°.

### EXAMPLE 4

### N⁵-[(2-hydroxy)-n-decyl]-homospermidine-trioxalate

A solution of 4.54 g (0.036 moles) of oxalic acid dihydrate in 30 ml of water was added whilst stirring to a solution of 6.19 g (0.012 moles) of N¹,N⁹-di-BOC-N⁵-[(2-hydroxy)-n-decyl]-homospermidine (example 3a) in 30 ml of ethanol. The reaction mixture was heated under reflux for 23 hours, and then concentrated by evaporation under vacuum. Purification of the crude product takes place analogously to example 1 on Amberlite XAD 1180 adsorber resin [eluant: H₂O and H₂O/isopropanol (19:1 or 4:1)]. After lyophilization, the title compound was obtained with a water content of 3.8%, R_{f}: 0.28 (solvent as for example 1).

### EXAMPLE 5

### N⁵-[(2-hydroxy)-n-hexadecyl]-homospermidine-tri-(toluene-4-sulfonate)

A mixture of 21.48 g (0.0358 moles) of N¹,N⁹-di-BOC-N⁵-[(2-hydroxy)-n-hexadecyl]-homospermidine and 20.43 g (0.1074 moles) of toluene-4-sulfonic acid monohydrate in 120 ml of water was heated for 11.5 hours at 70° whilst stirring, and subsequently concentrated to a volume of ca. 30 ml. Purification of the concentrate was effected analogously to example 1 on Amberlite XAD 1180 adsorber resin [eluant: H₂O and H₂O/isopropanol (4:1 or 3:2)]. The title compound was obtained as a lyophilizate with a water content of 2.8%,

R_{f}: 0.32 (solvent as in example 1).

The starting compound was produced as follows:

a) N¹,N⁹-di-BOC-N⁵-[(2-hydroxy)-n-hexadecyl]-homospermidine

15.91 g (0.0562 moles) of 1,2-hexadecene oxide (85%) were added to a solution of 13.48 g (0.0375 moles) of N¹,N⁹-di-BOC-homospermidine (example 3b) in 150 ml of ethanol, the reaction mixture was boiled under reflux for 20 hours and then concentrated by evaporation under vacuum. Purification of the oily crude product was effected by-flash chromatography on silica gel, whereby the eluants used were ethyl acetate/hexane mixtures (1:3 or 1:2 or 1:1) and ethyl acetate. The title compound was obtained in the form of an oil, R_{f}: 0.45 (solvent as in example 3a).

### EXAMPLE 6

### N⁵-[(2-hydroxy)-n-hexyl]-homospermidine Trioxalate

A solution of 4.6 g (0.0365 moles) of oxalic acid dihydrate in 40 ml of water was added to a solution of 5.6 g (0.01218 moles) of N¹,N⁹-di-BOC-N⁵-[(2-hydroxy)-n-hexyl]-homo-spermidine in 20 ml of ethanol, the reaction mixture was heated under reflux for 4.5 hours, and then concentrated by evaporation under vacuum. The crude product obtained was dissolved in methanol and precipitated by the dropwise addition of ether. Filtration was carried out and the title compound was recrystallized from ethanol/water, m.p. 85-90°.

The starting compound was produced as follows:

a) N¹,N⁹-di-BOC-N⁵-[(2-hydroxy)-n-hexyl]-homospermidine

1.80 g (0.018 moles) of 1,2-hexene oxide were added to a solution of 4.31 g (0.012 moles) of N¹,N⁹-di-BOC-homospermidine (example 3b) in 40 ml of ethanol, the reaction mixture was boiled under reflux for 22 hours and then concentrated by evaporation under vacuum. The oily residue was purified by flash chromatography on silica gel, using methylene chloride/methanol mixtures (99:1 or 19:1 or 9:1). The title compound was obtained in the form of an oil,

R_{f}: 0.32 (solvent as in example 3a).

### EXAMPLE 7

### N⁵-[(2-hydroxy)-n-butyl]-homospermidine-tri-(toluene-4-sulfonate)

A mixture of 6.39 g (0.0148 moles) of N¹,N⁹-di-BOC-N⁵-[(2-hydroxy)-n-butyl] homospermidine and 8.45 g (0.0444 moles) of toluene-4-sulfonic acid monohydrate in 30 ml of water was heated at 75° for 3.5 hours whilst stirring, then after cooling it was adjusted to pH 6 with 1N sodium hydroxide solution, and concentrated under vacuum. Purification of the concentrate was effected analogously to example 1 on Amberlite XAD 1180 adsorber resin [eluant: water and water/isopropanol (9:1)]. The title compound was obtained as a lyophilizate with a water content of 1.4%; R_{f}: 0.14 (solvent as in example 1).

The starting compound was produced as follows:

a) N¹,N⁹-di-BOC-N⁵-[(2-hydroxy)-n-butyl]-homospermidine

1.51 g (0.021 moles) of 1,2-butene oxide were added to a solution of 5.39 g (0.015 moles) of N¹,N⁹-di-BOC-homospermidine (example 3b) in 50 ml of ethanol. The reaction mixture was heated at 80° for 5 hours, then a further 0.36 g (0.005 moles) of 1,2-butene oxide were added, heating continued for 15 hours at 80°, and the mixture was concentrated by evaporation under vacuum. Purification of the crude product was effected by flash chromatography on silica gel, using methylene chloride/methanol mixtures (50:1 or 20:1 or 10:1). The title compound was obtained in the form of an oil, R_{f}: 0.20 (solvent as in example 3a).

### EXAMPLE 8

### N⁵-[(2-hydroxy)-n-octyl]-homospermidine Trioxalate

A solution of 3.64 g (0.0289 moles) of oxalic acid dihydrate in 36 ml of water was added whilst stirring to a solution of 4.7 g (0.00963 moles) of N¹,N⁹-di-BOC-N⁵-[(2-hydroxy)-n-octyl]-homospermidine in 12 ml of ethanol, the reaction mixture was heated at 90° for 4.5 hours, and then concentrated by evaporation under vacuum. After recrystallisation of the residue from ethanol, the title compound was obtained with a water content of 2.2%, m.p. 83-85°.

The starting compound was produced as follows:

a) N¹,N⁹-di-BOC-N⁵-[(2-hydroxy)-n-octyl]-homospermidine

2.31 g (0.018 moles) of 1,2-octene oxide were added to a solution of 5.39 g (0.015 moles) of N¹,N⁹-di-BOC-homospermidine (example 3b) in 50 ml of ethanol. The reaction mixture was heated at 80° for 15 hours, then a further 0.39 g (0.00304 moles) of 1,2-octene oxide were added, heating continued for 8 hours at 80°, and the mixture was concentrated by evaporation under vacuum. Purification of the crude product was effected analogously to example 7a. The title compound was obtained in the form of an oil, R_{f}: 0.35 (solvent as in example 3a).

### EXAMPLE 9

### N⁵-[(2-hydroxy)-n-hexadecyl]-N¹,N¹,N⁹,N⁹-tetramethyl-homospermidine-tri-(toluene-4-sulfonate)

11.8 ml (0.15 moles) of a 35% solution of formaldehyde in water and 0.75 g of palladium on activated carbon (10% Pd) were added to a solution of 2.83 g (0.003 moles) of N⁵-[(2-hydroxy)-n-hexadecyl]-homospermidine-tri-(toluene-4-sulfonate) (example 5) in 20 ml of water. Hydrogenation was carried out at room temperature until the hydrogen uptake has ended. Filtration was effected, the filtrate was concentrated by evaporation under vacuum, and the residue was partitioned between 2N sodium hydroxide solution and ethyl acetate. The organic phase which was washed with concentrated aqueous sodium chloride solution and dried over sodium sulfate was concentrated by evaporation, the residue dissolved in methanol and the methanolic solution adjusted to a pH value of 3 by adding 2N hydrochloric acid. After evaporation under vacuum and recrystallization of the residue from methanol/ether, the title compound was obtained, m.p. 236-239°.

### EXAMPLE 10

### N⁴-[(2-hydroxy)-n-decyl]-N¹,N¹,N⁸,N⁸-tetramethyl-spermidine Trioxalate

1.6 g (0.002745 moles) of N⁴-[(2-hydroxy)-n-decyl]-spermidine trioxalate (example 27) were reacted analogously to example 9 with 11.8 ml (0.15 moles) of a 35% solution of formaldehyde in water. After concentrating by evaporation, the residue was crystallized from acetonitrile. After recrystallization from methanol/acetonitrile, the title compound was obtained with a water content of 1.69%, m.p. 118-121°.

### EXAMPLE 11

### N¹,N⁴-bis-(3-aminopropyl)-N¹,N⁴-bis[(2-hydroxy)-n-hexadecyl]-1,4-diamino-trans-2-butene-trioxalate

A mixture of 2.7 g (0.00306 moles) of N¹,N⁴-bis[3-BOC-aminopropyl]-N¹,N⁴-bis[(2-hydroxy)-n-hexadecyl]-1,4-diamino-trans-2-butene, 1.16 g (0.0092 moles) of oxalic acid dihydrate and 30 ml of water was reacted analogously to example 13 (duration of reaction: 18 hours). The title compound, which was recrystallized a second time from water/acetonitrile, contains 2.3% water, m.p. 165° (decomp.).

The starting compounds were produced as follows:

a) N¹,N⁴-bis[3-BOC-aminopropyl]-N¹,N⁴-bis[(2-hydroxy)-n-hexadecyl]-1,4-diamino-trans-2-butene

A mixture of 2 g (0.005 moles) of N¹,N⁴-bis[3-BOC-aminopropyl]-1,4-diamino-trans-2-butene, 3.54 g (0.0125 moles) of 1,2-hexadecene oxide (85%) and 40 ml of ethanol was boiled under reflux for 24 hours and subsequently concentrated by evaporation under vacuum. After purification of the residue by flash chromatography on silica gel, using methylene chloride/methanol mixtures (100:1 or 25:1), the title compound was obtained in the form of an oil, which solidified into crystalline form after a short time, m.p. 85-87°.

b) N¹,N⁴-bis[3-BOC-aminopropyl]-N¹-BOC-1,4-diamino-trans-2-butene and N¹,N⁴-bis[3-BOC-aminopropyl]-1,4-diamino-trans-2-butene

A solution of 46.18 g (0.1875 moles) of 2-(BOC-oxyimino)-2-phenylacetonitrile in 150 ml of THF was added dropwise whilst stirring, over the course of 3 hours, and in a nitrogen atmosphere, to a solution, cooled to 0-5°, of 15.02 g (0.075 moles) of N¹,N⁴-bis(3-aminopropyl)-1,4-diamino-trans-2-butene in 100 ml of THF. The reaction mixture was stirred for a further 3.5 days at room temperature, then concentrated by evaporation under vacuum and the residue was separated by flash chromatography on silica gel, using methylene chloride/methanol mixtures (39:1 or 9:1) and mixtures of methylene chloride/methanol/30% aqueous ammonia solution (90:10:0.25 or 10:5:1). The first title compound, N¹,N⁴-bis[3-BOC-aminopropyl]-N′-BOC-1,4-diamino-trans-2-butene, was thereby obtained in the form of an oil, R_{f}: 0.87 (solvent as in example 1a), and the second title compound, N¹,N⁴-bis[3-BOC-aminopropyl]-1,4-diamino-trans-2-butene, was also obtained in the form of an oil, R_{f}: 0.26 (solvent as in example 1a).

### EXAMPLE 12

### N¹,N⁴-bis(3-aminopropyl)-N′-[(2-hydroxy)-n-hexadecyl]-1,4-diamino-trans-2-butene-tetraoxalate

The title compound was obtained analogously to example 11, from 1.58 g (0.00213 moles) of N¹,N⁴-bis[3-BOC-aminopropyl]-N′-BOC-N⁴-[(2-hydroxy)-n-hexadecyl]-1,4-diamino-trans-2-butene, 1.075 g (0.00853 moles) of oxalic acid dihydrate and 20 ml of water. M.p. 185° (decomp.).

The starting compound was produced as follows:

a) N¹,N⁴-bis[3-BOC-aminopropyl]-N′-BOC-N⁴-[(2-hydroxy-n-hexadecyl]-14-diamino-trans-2-butene

The title compound was obtained in the form of an oil, analogously to example 11a, from 2.5 g (0.005 moles) of N¹,N⁴-bis[3-BOC-aminopropyl]-N′-BOC-1,4-diamino-trans-2-butene and 1.77 g (0.00626 moles) of 1,2-hexadecene oxide (85%). Rf: 0.59 (solvent as in example 3a).

### EXAMPLE 13

### N⁴-[(2-hydroxy)-n-hexadecyl]-N⁹-octyl-spermine Tetraoxalate

A mixture of 1.08 g (0.00143 moles) of N¹,N¹²-di-BOC-N⁴-[(2-hydroxy)-n-hexadecyl]-N⁹-n-octyl-spermine, 0.721 g (0.00577 moles) of oxalic acid dihydrate and 20 ml of water was heated under reflux for 16 hours and subsequently mixed with acetonitrile (until slight turbidity occurs). The product which precipitated upon cooling was filtered, washed with acetonitrile and dried under a high vacuum at 100°. The title compound obtained contained 1.6% water, m.p. 170-180° (decomp.).

The starting compounds were produced as follows:

a) N¹,N¹²-di-BOC-N⁴-[(2-hydroxy-n-hexadecyl]-N-9-n-octyl-spermine

A mixture of 1.3 g (0.00202 moles) of N¹,N¹²-di-BOC-N⁴-[(2-hydroxy)-n-hexadecyl]-spermine, 0.444 g (0.0023 moles) of 1-bromoctane, 1.1 g (0.00796 moles) of potassium carbonate and 20 ml of acetonitrile was heated under reflux for 16 hours. A further 0.089 g (0.00046 moles) of 1-bromoctane were added to the reaction mixture, and heating continued under reflux for a further 6 hours. After the further addition of 0.089 g (0.00046 moles) of 1-bromoctane and heating under reflux for 14 hours, the reaction mixture was concentrated by evaporation under vacuum. Purification of the residue was carried out using flash chromatography on silica gel, using methylene chloride/methanol mixtures (50:1 or 9:1) and a mixture of methylene chloride/methanol/30% aqueous ammonia solution (90:10:0.25). The title compound was obtained in the form of an oil,

R_{f}: 0.76 [solvent: toluene/isopropanol/30% aqueous ammonia solution (70:29:1)].

b) N¹,N¹²-di-BOC-N⁴-[(2-hydroxy)-n-hexadecyl]-spermine and N¹,N¹²-di-BOC-N⁴,N⁹-bis-[(2-hydroxy)-n-hexadecyl]-spermine

3.21 g (0.01136 moles) of 1,2-hexadecene oxide (85%) were added to a solution of 3.98 g (0.00989 moles) of N¹,N¹²-di-BOC-spermine in 40 ml of ethanol, the reaction mixture was boiled under reflux for 20 hours and then concentrated by evaporation under vacuum. Upon chromatography of the crude mixture on silica gel, using methylene chloride/methanol mixtures (100:1 or 9:1), first of all N¹,N²-di-BOC-N⁴,N⁹-bis-[(2-hydroxy)-n-hexadecyl]-spermine elutes, R_{f}: 0.31 (solvent as in example 3a), and then using mixtures of methylene chloride/methanol/30% aqueous ammonia solution (90:10:0.25 or 40:10:0.5), N¹,N¹²-di-BOC-N⁴-[(2-hydroxy)-n-hexadecyl]-spermine elutes, R_{f}: 0.07 (solvent as in example 13a).

c) N¹,N⁹,N¹²-tri-BOC-spermine and N¹,N¹²-di-BOC-spermine

50 g (0.2471 moles) of spermine were dissolved in 300 ml of THF under a nitrogen atmosphere, whilst stirring, and then at 0-5°, a solution of 134 g (0.544 moles) of 2-(BOC-oxyimino)-2-phenylacetonitrile in 500 ml of THF was added dropwise over the course of one hour. The reaction mixture was stirred for a further 16 hours at room temperature and then concentrated by evaporation under vacuum. Upon separation of the reaction mixture by flash chromatography on silica gel, using methylene chloride, methylene chloride/methanol mixtures (97.5:2.5 or 9:1) and mixtures of methylene chloride/methanol/30% aqueous ammonia solution (90:10:0.5 or 20:10:1), the following were obtained: oily N¹,N⁹,N¹²-tri-BOC-spermine [see J. Org. Chem. 50, 5735 (1985)], Rf: 0.78 (solvent as in example 1a), slightly impure N¹,N¹²-di-BOC-spermine, m.p. 86-88° and pure N¹,N¹²-di-BOC-spermine, m.p. 91-92°.

d) N¹,N¹²-di-BOC-spermine may also be produced in the following manner:

18.4 g (0.0196 moles) of N¹,N⁴,N⁹,N¹²-tetrakis(benzyloxycarbonyl)-N¹,N¹²-di-BOC-spermine were dissolved in 200 ml of methanol. After adding 1.8 g of palladium on activated carbon (10% Pd), hydrogenation was carried out at room temperature until the hydrogen uptake had ended. The solution was filtered and the filtrate was concentrated by evaporation under vacuum. The oily title compound, R_{f}: 0.09 (solvent as in example 1a), which gradually changed into a crystalline state, was identical to the N¹,N¹²-di-BOC-spermine obtained according to example 13c.

e) N¹,N⁴,N⁹,N¹²-tetrakis(benzyloxycarbonyl)-N¹,N¹²-di-BOC-spermine

0.57 g (0.00466 moles) of 4-dimethylaminopyridine and a solution of 11.24 g (0.0515 moles) of di-(tert.-butyl)-dicarbonate in 25 ml of acetonitrile were added whilst stirring to a solution of 17.3 g (0.0234 moles) of N¹,N⁴,N⁹,N¹²-tetrakis(benzyloxycarbonyl)-spermine in 40 ml of acetonitrile. The reaction mixture was stirred at room temperature for 18 hours, subsequently concentrated by evaporation, and the residue was purified by flash chromatography on silica gel, using hexane/ethyl acetate mixtures (4:1 or 3:1 or 2:1 or 1:1). The title compound was obtained in the form of an oil, Rf: 0.38 [solvent: ethyl acetate/hexane (1:1)].

f) N¹,N⁴,N⁹,N¹²-tetrakis(benzyloxycarbonyl-spermine

82.82 ml (0.25 moles) of chloroformic acid benzyl ester (50% in toluene) were added dropwise at room temperature, over the course of one hour, to a well stirred solution of 10.12 g (0.05 moles) of spermine and 39.75 g (0.375 moles) of sodium carbonate in 200 ml of water. The reaction mixture was stirred for 4 hours, filtered and the organic phase separated. This phase was washed with water and with concentrated aqueous sodium chloride solution, dried over sodium sulfate, and concentrated by evaporation under vacuum. Purification of the residue was effected by flash chromatography on silica gel, using ethyl acetate/hexane mixtures (1:3 or 1:2 or 1:1). The title compound was obtained in the form of an oil, R_{f}: 0.37 [solvent: ethyl acetate/hexane 2:1)].

### EXAMPLE 14

### N⁵-(2-hydroxyethyl)-homospermidine Trioxalate

The title compound was obtained analogously to example 8, from 2.6 g (0.00644 moles) of N¹,N⁹-di-BOC-N⁵-(2-hydroxyethyl)-homospermidine and 2.435 g (0.0193 moles) of oxalic acid dihydrate. M.p. 127-130°.

The starting compound was produced as follows:

a) N¹,N⁹-di-BOC-N⁵-(2-hydroxyethyl)-homospermidine

3.2 g (0.0726 moles) of ethylene oxide were passed into a solution, cooled to 5°, of 7.19 g (0.02 moles) of N¹,N⁹-di-BOC-homospermidine in 25 ml of methanol over the course of ca. 20 minutes. The reaction mixture was stirred for 21 hours at room temperature and subsequently concentrated by evaporation under vacuum. Purification of the residue was effected by flash chromatography on silica gel, using methylene chloride/methanol mixtures (30:1 or 10:1 or 5:1). The title compound was obtained in the form of an oil, R_{f}: 0.07 (solvent as in example 3a).

### EXAMPLE 15

### N⁴,N⁹-bis[(2-hydroxy)-n-octyl]-spermine Tetraoxalate

A solution of 1.26 g (0.01 moles) of oxalic acid dihydrate in 10 ml of water was added to a solution of 1.65 g (0.0025 moles) of N¹,N¹²-di-BOC-N⁴,N⁹-bis[(2-hydroxy)-n-octyl]-spermine in 5 ml of ethanol. The reaction mixture was stirred for 9 hours at 90°, then concentrated by evaporation under vacuum, and the residue was crystallized from methanol/ether. The title compound obtained melted at 126-129°.

The starting compound was produced as follows:

a) N¹,N¹²-di-BOC-N⁴,N⁹-bis[(2-hydroxy)-n-octyl]-spermine

A mixture of 1.01 g (0.0025 moles) of N¹,N¹²-di-BOC-spermine, 0.96 g (0.0075 moles) of 1,2-octene oxide and 15 ml of ethanol was stirred for 21 hours at 85° and subsequently concentrated by evaporation under vacuum. Purification of the residue was effected by flash chromatography on silica gel, using methylene chloride/methanol mixtures (19:1 or 9:1). The title compound was obtained in the form of an oil, R_{f}: 0.23 (solvent as in example 3a).

### EXAMPLE 16

### N⁴,N⁹-bis[(2-hydroxy)-n-decyl]-spermine Tetraoxalate

A solution of 3.73 g (0.0296 moles) of oxalic acid dihydrate in 10 ml of water was added to a solution of 5.3 g (0.00741 moles) of N¹,N¹²-di-BOC-N⁴,N⁹-bis[(2-hydroxy)-n-decyl]-spermine in 10 ml of ethanol. The reaction mixture was stirred for 10 hours at 90°, then concentrated by evaporation, and the residue crystallized from methanol/ether. The title compound obtained melts at 175-177°.

The starting compound was produced as follows:

a) N¹,N¹²-di-BOC-N⁴ N⁹-bis[(2-hydroxy)-n-decyl]-spermine

A mixture of 3.22 g (0.008 moles) of N¹,N¹²-di-BOC-spermine, 3.75 g (0.024 moles) of 1,2-decene oxide and 32 ml of ethanol was stirred for 19 hours at 80° and subsequently concentrated by evaporation under vacuum. Purification of the residue was effected by flash chromatography on silica gel, using methylene chloride and methylene chloride/methanol mixtures (50:1 or 19:1 or 9:1). The title compound was obtained in the form of an oil, R_{f}: 0.25 (solvent as in example 3a).

### EXAMPLE 17

### N⁴,N⁹-bis[(2-hydroxy)-n-dodecyl]-spermine-tetraoxalate

The title compound was obtained analogously to example 15, but maintaining the reaction for 10 hours, from 1.7 g (0.0022 moles) of N¹,N¹²-di-BOC-N⁴,N⁹-bis[(2-hydroxy)-n-dodecyl]-spermine and 1.11 g (0.0088 moles) of oxalic acid dihydrate. M.p. 187° (decomp.).

The starting compound was produced as follows:

a) N¹,N¹²-di-BOC-N⁴,N⁹-bis[(2-hydroxy)-n-dodecyl]-spermine

1.01 g (0.0025 moles) of N¹,N¹²-di-BOC-spermine and 1.38 g (0.0075 moles) of 1,2-dodecene oxide were reacted analogously to example 15a (duration of reaction: 22 hours). The title compound which was purified by flash chromatography on silica gel [eluant: methylene chloride/methanol (99:1 or 19:1)] was obtained in the form of an oil, R_{f}: 0.27 (solvent as in example 3a).

### EXAMPLE 18

### N⁴,N⁹-bis[(2-hydroxy)-n-tetradecyl]-spermine Tetraoxalate

1.82 g (0.0022 moles) of N¹,N¹²-di-BOC-N⁴,N⁹-bis[(2-hydroxy)-n-tetradecyl]-spermine and 1.11 g (0.0088 moles) of oxalic acid dihydrate were reacted analogously to example 15, but maintaining the reaction for 11.5 hours. After crystallization from methanol/water, the title compound decomposed at 170°.

The starting compound was produced as follows:

a) N¹,N¹²-di-BOC-N⁴,N⁹-bis[(2-hydroxy)-n-tetradecyl]-spermine

1.01 g (0.0025 moles) of N¹,N¹²-di-BOC-spermine and 1.874 g (0.0075 moles) of 1,2-tetradecene oxide (85%) were reacted analogously to example 15a (duration of reaction: 18.5 hours). The title compound which was purified by flash chromatography on silica gel [eluant: methylene chloride/methanol (99:1 or 49:1 or 19:1 or 9:1)] was obtained in the form of an oil, R_{f}: 0.30 (solvent as in example 3a).

### EXAMPLE 19

### N⁴,N⁹-bis[(2-hydroxy)-n-hexadecyl]-spermine Tetraoxalate

A mixture of 3.53 g (0.004 moles) of N¹,N¹²-di-BOC-N⁴,N⁹-bis[(2-hydroxy)-n-hexadecyl]-spermine, 3.04 g (0.016 moles) of toluene-4-sulfonic acid monohydrate and 20 ml of water was stirred for 19 hours at 70°, subsequently concentrated by evaporation under vacuum, and the residue partitioned between 2N sodium hydroxide solution and chloroform. After washing the organic phase with concentrated sodium chloride solution, drying over sodium sulfate and concentrating by evaporation under vacuum, crude N⁴,N⁹-bis[(2-hydroxy)-n-hexadecyl]-spermine was obtained, which was dissolved in 32 ml of ethanol and mixed, whilst stirring, with a solution of 2.0 g (0.016 moles) of oxalic acid dihydrate in 32 ml of ethanol, whereby the title compound precipitated in crystalline form. The crystallizate which was washed with ethanol and dried under a high vacuum melted at 140° under decomposition.

The starting compound was produced as follows:

a) N¹,N¹²-di-BOC-N⁴,N⁹-bis[(2-hydroxy)-n-hexadecyl]-spermine

2.02 g (0.005 moles) of N¹,N¹²-di-BOC-spermine and 4.24 g (0.015 moles) of 1,2-hexadecene oxide (85%) were reacted analogously to example 15a (duration of reaction: 8 hours). The title compound which was purified by flash chromatography on silica gel, using ethyl acetate/hexane mixtures (1:3 or 1:2 or 1:1), using ethyl acetate and using an ethyl acetate/methanol mixture (19:1), was obtained in the form of an oil, R_{f}: 0.31 (solvent as in example 3a).

### EXAMPLE 20

### N⁴-[(2-hydroxy)-n-hexadecyl]-spermine-tetra(toluene-4-sulfonate)

A mixture of 5.94 g (0.008 moles) of N¹,N⁹,N¹²-tri-BOC-N⁴-[(2-hydroxy)-n-hexadecyl]-spermine, 6.09 g (0.032 moles) of toluene-4-sulfonic acid monohydrate and 35 ml of water was reacted analogously to example 5 (duration of reaction: 2.5 hours). After purification on Amberlite XAD 1180 adsorber resin, using water and water/isopropanol mixtures (9:1 or 4:1 or 3:2), the title compound was obtained as a lyophilizate with a water content of 2.24%,

R_{f}: 0.07 (solvent as in example 1).

The starting compound was produced as follows:

a) N¹,N⁹,N¹²-tri-BOC-N-4-[(2-hydroxy)-n-hexadecyl]-spermine N⁴-[(2-hydroxy)-n-decyl]-spermine Tetraoxalate

5.02 g (0.01 moles) of N¹,N⁹,N¹²-tri-BOC-spermine and 4.24 g (0.015 moles) of 1,2-hexadecene oxide (85%) were reacted analogously to example 15a (duration of reaction: 10.5 hours). The title compound which was purified by flash chromatography on silica gel, using ethyl acetate/hexane mixtures (1:3 or 1:1) and using ethyl acetate, was obtained in the form of an oil, R_{f}: 0.52 (solvent as in example 3a).

A mixture of 4.05 g (0.00615 moles) of N¹,N⁹,N¹²-tri-BOC-N⁴-[(2-hydroxy)-n-decyl]-spermine, 3.1 g (0.0246 moles) of oxalic acid dihydrate, 8 ml of ethanol and 8 ml of water was reacted analogously to example 15 (duration of reaction: 12.5 hours). After crystallisation from ethanol/ether, the title compound decomposed at 135-155°.

The starting compound was produced as follows:

a) N¹,N⁹,N¹²-tri-BOC-N⁴-[(2-hydroxy)-n-decyl]-spermine

A mixture of 4.02 g (0.008 moles) of N¹,N⁹,N¹²-tri-BOC-spermine, 1.875 g (0.012 moles) of 1,2-decene oxide and 40 ml of ethanol was stirred for 20 hours at 80° and subsequently concentrated by evaporation under vacuum. After purifying the residue by flash chromatography on silica gel, using methylene chloride and methylene chloride/methanol mixtures (50:1 or 19:1 or 9:1), the title compound was obtained in the form of an oil,

R_{f}: 0.40 (solvent as in example 3a).

### EXAMPLE 22

### N⁴-[(R)-(2-hydroxy)-n-hexadecyl]-spermine Tetraoxalate

A mixture of 5.6 g (0.00754 moles) of N¹,N⁹,N¹²-tri-BOC-N⁴-[(R)-(2-hydroxy)-n-hexadecyl]-spermine, 3.8 g (0.03016 moles) of oxalic acid dihydrate and 50 ml of water was reacted analogously to Example 13 (duration of reaction: 18 hours). The title compound obtained decomposes at 200-205°, [α]_{D}²⁰=−7.4±1.7° (c=0.5% in H₂O).

The starting compound was produced as follows:

a) N¹,N⁹,N¹²-tri-BOC-N⁴-[(R)-(2-hydroxy)-n-hexadecyl]-spermine

A mixture of 7.04 g (0.014 moles) of N¹,N⁹,N¹²-tri-BOC-spermine, 4.06 g (0.0169 moles) of (R)-1,2-hexadecene oxide (Nippon Mining Company, Ltd.) and 30 ml of ethanol was stirred for 15 hours at 80° and subsequently concentrated by evaporation under vacuum. After purifying the residue by flash chromatography on silica gel, using methylene chloride and a methylene chloride/methanol mixture (19:1), the title compound was obtained in the form of an oil, R_{f}: 0.52 (solvent as in example 3a).

### EXAMPLE 23

### N⁴-(2-hydroxyethyl)-spermidine Trioxalate

A mixture of 2.73 g (0.007 moles) of N¹,N⁸-di-BOC-N⁴-(2-hydroxyethyl)-spermidine, 2.65 g (0.021 moles) of oxalic acid dihydrate, 10 ml of ethanol and 30 ml of water was stirred for 4.5 hours at 90°. The reaction mixture which was still warm was mixed with ethanol (until slight turbidity occurs) and was then cooled to 0°, whereby the title compound results in crystalline form, m.p. 153-156° (decomp.).

The starting compound was produced as follows:

a) N¹,N⁸-di-BOC-N⁴-(2-hydroxyethyl)-spermidine

The title compound was obtained in the form of an oil analogously to example 14a, from 6.91 g (0.02 moles) of N¹,N⁸-di-BOC-spermidine and 3.2 g (0.0726 moles) of ethylene oxide, after purifying the crude product on silica gel using methylene chloride/methanol mixtures (19:1 or 9:1 or 4:1). R_{f}: 0.76 (solvent as in example 1a).

### EXAMPLE 24

### N⁴-(2-hydroxy)-n-hexadecyl]-spermidine-tri(toluene-4-sulfonate)

A mixture of 6.21 g (0.0106 moles) of N¹,N⁸-di-BOC-N⁴-[(2-hydroxy)-n-hexadecyl]-spermidine, 6.05 g (0.0318 moles) of toluene-4-sulfonic acid monohydrate and 30 ml of water was stirred for 2 hours at 75°. After purification of the reaction mixture by chromatography on Amberlite XAD 1180 absorber resin [eluant: water and water/isopropanol (4:1 or 3:2)] and subsequent lyophilization of the product-containing fractions, the title compound was obtained as the lyophilizate with a water content of 2.2%, R_{f}: 0.26 (solvent as in example 1).

The starting compound was produced as follows:

a) N¹,N⁸-di-BOC-N⁴-[(2-hydroxy)-n-hexadecyl]-spermidine

10.61 g (0.0375 moles) of 1,2-hexadecene oxide (85%) were added to a solution of 8.64 g (0.025 moles) of N¹,N⁸-di-BOC-spermidine in 100 ml of ethanol. The reaction mixture was boiled under reflux for 15 hours, a further 1.7 g (0.006 moles) of 1,2-hexadecene oxide were added, the mixture was boiled under reflux for a further 7 hours and then concentrated by evaporation under vacuum. Purification of the crude product was effected by flash chromatography on silica gel, using ethyl acetate/hexane mixtures (1:2 or 1:1) and using ethyl acetate. The title compound was obtained in the form of an oil, R_{f}: 0.85 (solvent as in example 1a).

### EXAMPLE 25

### N⁴-[(2-hydroxy)-n-hexadecyl]-norspermidine-tri(toluene-4-sulfonate)

The title compound was obtained as the lyophilizate with a water content of 1.4%, analogously to example 24, from 5.72 g (0.01 moles) of N¹,N⁷-di-BOC-N⁴-[(2-hydroxy)-n-hexadecyl]-norspermidine and 5.71 g (0.03 moles) of toluene-4-sulfonic acid monohydrate, R_{f}: 0.24 (eluant as in example 1).

The starting compound was produced as follows:

a) N¹,N⁷-di-BOC-N⁴-[(2-hydroxy)-n-hexadecyl]-norspermidine

6.56 g (0.0232 moles) of 1,2-hexadecene oxide (85%) were added to a solution of 6.4 g (0.0193 moles) of N¹,N⁷-di-BOC-norspermidine (Hansen et al., Synthesis 1982:404) in 75 ml of ethanol, and the reaction mixture was boiled under reflux for 17.5 hours. After adding a further 2.55 g (0.009 moles) of 1,2-hexadecene oxide (85%), the reaction mixture was again boiled under reflux for 22 hours and then worked up analogously to example 24a. The title compound was obtained in the form of an oil, R_{f}: 0.79 (solvent as in example 1a).

### Example 26

### N⁴-[(2-hydroxy)-n-decyl]-norspermidine Trioxalate

The title compound was obtained analogously to example 13, from 3.2 g (0.00656 moles) of N¹,N⁷-di-BOC-N⁴-[(2-hydroxy)-n-decyl]-norspermidine, 2.48 g (0.0197 moles) of oxalic acid dihydrate and 25 ml of water. M.p. 174-179° (decomp.).

The starting compound was produced as follows:

a) N¹,N⁷-di-BOC-N⁴-[(2-hydroxy)-n-decyl]-norspermidine

The title compound was obtained in the form of an oil analogously to example 22a, from 2.49 g (0.0075 moles) of N¹,N⁷-di-BOC-norspermidine, 1.47 g (0.0094 moles) of 1,2-decene oxide and 25 ml of ethanol. After a short time, the compound solidifies into crystalline form, m.p. 52-54°.

### EXAMPLE 27

### N⁴-[(2-hydroxy)-n-decyl]-spermidine-trioxalate

A mixture of 3.19 g (0.00636 moles) of N¹,N⁸-di-BOC-N⁴-[(2-hydroxy)-n-decyl]-spermidine, 2.405 g (0.01908 moles) of oxalic acid dihydrate and 25 ml of water was boiled under reflux for 15 hours and subsequently concentrated by evaporation under vacuum. After crystallisation of the residue from acetone, the title compound was obtained with a-water content of 1.9%.

M.p. 170-173° (decomp.).

The starting compound was produced as follows:

a) N¹,N⁸-di-BOC-N⁴-[(2-hydroxy)-n-decyl]-spermidine

The title compound was obtained in the form of an oil, analogously to example 22a, from 2.59 g (0.0075 moles) of N¹,N⁸-di-BOC-spermidine, 1.47 g (0.0094 moles) of 1,2-decene oxide and 25 ml of ethanol. R_{f}: 0.50 (solvent as in example 3a).

### Example 28

### N⁴,N⁹-bis[(S)-(2-hydroxy)-n-decyl]-spermine Tetraoxalate

A mixture of 2.72 g (0.0038 moles) of N¹,N¹²-di-BOC-N⁴,N⁹-bis[(S)-(2-hydroxy)-n-decyl]-spermine, 1.916 g (0.0152 moles) of oxalic acid dihydrate and 30 ml of water was boiled under reflux for 15 hours. Acetone was added to the reaction mixture whilst it was still hot (and until slight turbidity occured), and the mixture was then slowly cooled to 0°, whereby the title compound precipitated in crystalline form. After filtration, washing the crystallizate with acetone and drying under a high vacuum, the title compound was obtained, m.p. 175-177° (decomp.), [α]_{D}²⁰=+13.1°±0.7° (c=1.47%, H₂O).

The starting compound was produced as follows:

a) N¹,N¹²-di-BOC-N¹,N⁹-bis[(S)-(2-hydroxy)-n-decyl]-spermine

A mixture of 2.013 g (0.005 moles) of N¹,N¹²-di-BOC-spermine, 2.34 g (0.015 moles) of (S)-1,2-decene oxide and 20 ml of ethanol was boiled under reflux for 15 hours and subsequently concentrated by evaporation under vacuum. After purification of the residue by flash chromatography on silica gel, using methylene chloride/methanol mixtures (99:1 or 49:1 or 19:1 or 9:1), the title compound was obtained in the form of an oil,

R_{f}: 0.25 (solvent as in example 3a), [α]_{D}²⁰=+52.84 (c=1.552%, hexane).

### EXAMPLE 29

### N⁴,N⁹-bis[(R)-(2-hydroxy)-n-decyl]-spermine Tetraoxalate

The title compound was obtained analogously to example 28, from 2.72 g (0.0038 moles) of N¹,N¹²-di-BOC-N⁴,N⁹-bis[(R)-(2-hydroxy)-n-decyl]-spermine and 1.916 g (0.0152 moles) of oxalic acid dihydrate. M.p. 175-177° (decomp.), [α]_{D}²⁰=−14.1°±0.7° (c=1.43%, H₂O).

The starting compound was produced as follows:

a) N¹,N¹²-di-BOC-N⁴,N⁹-bis[(R)-(2-hydroxy)-n-decyl]-spermine

The title compound was obtained in the form of an oil, analogously to example 28a, from 2.013 g (0.005 moles) of N¹,N¹²-di-BOC-spermine and 2.34 g (0.015 moles) of (R)-1,2-decene oxide, R_{f}: 0.25 (solvent as in example 3a), [α]_{D}²⁰=−52.84° (c=1.268%, hexane).

### EXAMPLE 30

### N¹,N⁸-bis(3-aminopropyl)-N¹-[(2-hydroxy)-n-hexadecyl]-1,8-diamino-octane Tetraoxalate

The title compound was obtained analogously to example 13, but with a reaction time of 20 hours, from 2.84 g (0.00355 moles) of N¹,N⁸-bis(3-BOC-aminopropyl)-N′-BOC-N⁸-[(2-hydroxy)-n-hexadecyl]-1,8-diamino-octane, 1.79 g (0.0142 moles) of oxalic acid dihydrate and 30 ml of water. M.p. 165-170° (decomp.).

The starting compound was produced as follows:

a) N¹,N⁸-bis(3-BOC-aminopropyl)-N¹-BOC-N⁸-[(2-hydroxy)-n-hexadecyl]-1,8-diamino-octane

4.8 g (0.00859 moles) of N¹,N⁸-bis(3-BOC-aminopropyl)-N′-BOC-1,8-diamino-octane and 2.91 g (0.0103 moles) of 1,2-hexadecene oxide (85%) in 30 ml of ethanol were reacted analogously to example 21a (duration of reaction 16 hours). The title compound which was purified by flash chromatography on silica gel, using methylene chloride and a methylene chloride/methanol mixture (20:1) was obtained in the form of an oil,

R_{f}: 0.60 (solvent as in example 3a).

b) N¹,N⁸-bis(3-BOC-aminopropyl)-N¹-BOC-18-diamino-octane and N¹,N⁸-bis(3-BOC-aminopropyl)-1,8-diamino-octane

A solution of 36.94 g (0.15 moles) of 2-(BOC-oxyimino)-2-phenylacetonitrile in 120 ml of THF was added dropwise whilst stirring, over the course of 1.5 hours, and under a nitrogen atmosphere, to a solution, cooled to 0-5°, of 15.51 g (0.06 moles) of N¹,N⁸-bis(3-aminopropyl)-1,8-diamino-octane [see Pestic. Sci., 485-490 (1973)] in 100 ml of THF. The reaction mixture was stirred for a further 16 hours at room temperature, then concentrated by evaporation under vacuum, and the residue was separated by flash chromatography on silica gel, using methylene chloride/methanol mixtures (100:1 or 50:1 or 20:1 or 10:1) and mixtures of methylene chloride/methanol/30% aqueous ammonia solution (90:10:0.5 or 90:15:0.5 or 40:10:1). The following were thereby obtained: the first title compound, N¹,N⁸-bis(3-BOC-aminopropyl)-N¹-BOC-1,8-diamino-octane, in the form of an oil, R_{f}: 0.81 (solvent as in example 1a), as well as the second compound, N¹,N⁸-bis(3-BOC-aminopropyl)-1,8-diamino-octane, m.p. 67-700, R_{f}: 0.26 (solvent as in example 1a).

### EXAMPLE 31

### N¹,N⁸-bis(3-aminopropyl)-N′-[(R)-(2-hydroxy)-n-hexadecyl]-1,8-diamino-octane Tetraoxalate

The title compound was obtained analogously to example 13, but maintaining the reaction for 21 hours, from 3.71 g (0.00464 moles) of N¹,N⁸-bis(3-BOC-aminopropyl)-N¹-BOC-N⁸-[(R)-(2-hydroxy)-n-hexadecyl]-1,8-diamino-octane, 2.34 g (0.01856 moles) of oxalic acid dihydrate and 35 ml of water. M.p. 165-170° (decomp.), [α]_{D}²⁰=−7.2°±1.6° (c=0.5%, H₂O).

The starting compound was produced as follows:

a) N¹,N⁸-bis(3-BOC-aminopropyl)-N¹-BOC-N⁸-[(R)-(2-hydroxy)-n-hexadecyl]-1,8-diamino-octane

The title compound was obtained in the form of an oil, analogously to example 30a, from 5 g (0.00895 moles) of N¹,N⁸-bis(3-BOC-aminopropyl)-N′-BOC-1,8-diamino-octane (example 30b), 2.58 g (0.01073 moles) of (R)-1,2-hexadecene oxide and 30 ml of ethanol. R_{f}: 0.60 (solvent as in example 3a).

### EXAMPLE 32

### N¹,N¹²-bis(3-aminopropyl)-N¹,N¹²-bis[(2-hydroxy)-n-hexadecyl]-1,12-diamino-dodecane Tetraoxalate

The title compound was obtained analogously to example 13, but maintaining the reaction for 40 hours, from 1.3 g (0.001305 moles) of N¹,N¹²-bis(3-BOC-aminopropyl)-N¹,N¹²-bis[(2-hydroxy)-n-hexadecyl]-1,12-diamino-dodecane, 0.66 g (0.00523 moles) of oxalic acid dihydrate and 20 ml of water. M.p. 115-118°.

The starting compound was produced as follows:

a) N¹,N¹²-bis(3-BOC-aminopropyl)-N¹,N¹²-bis[(2-hydroxy)-n-hexadecyl]-1,12-diamino-dodecane

1.1 g (0.002137 moles) of N¹,N¹²-bis(3-BOC-aminopropyl)-1,12-diamino-dodecane and 1.45 g (0.00513 moles) of 1,2-hexadecene oxide (85%) in 25 ml of ethanol were reacted analogously to example 15a (duration of reaction: 18 hours). The title compound which was purified by flash chromatography on silica gel, using methylene chloride/methanol mixtures (50:1 or 25:1 or 10:1) was obtained in the form of an oil, R_{f}: 0.91 (solvent as in example 1a).

b) N¹,N¹²-bis(3-BOC-aminopropyl)-N-BOC-1,12-diamino-dodecane and N¹,N¹²-bis(3-BOC-aminopropyl)-1,12-diamino-dodecane

36.1 ml (0.195 moles) of a 5.4 molar methanolic solution of sodium methylate was added whilst stirring at room temperature and under a nitrogen atmosphere to a suspension of 23.9 g (0.0519 moles) of 1,12-bis(3-aminopropyl)-1,12-diamino-dodecane-tetrahydrochloride [J. Med. Chem. 7, 710 (1964)] in 130 ml of THF. After stirring for 20 minutes, the reaction mixture was cooled to 0°, and over the course of 1 hour, was mixed with a solution of 38.39 g (0.1559 moles) of 2-(BOC-oxyimino)-2-phenylacetonitrile in 130 ml of THF. Stirring continued for 15 hours at room temperature, the solution was filtered and the filtrate was concentrated by evaporation under vacuum. The residue was separated by flash chromatography on silica gel, using methylene chloride/methanol mixtures (50:1 or 25:1 or 16:1 or 10:1) and mixtures of methylene chloride/methanol/30% aqueous ammonia solution (90:10:0.5 or 90:15:0.5 or 40:10:1). The following were thereby obtained: the first title compound, N¹,N¹²-bis(3-BOC-aminopropyl)-N¹-BOC-1,12-diamino-dodecane, in the form of an oil, R_{f}: 0.85 (solvent as in example 1a), as well as the second title compound, N¹,N¹²-bis(3-BOC-aminopropyl)-1,12-diamino-dodecane, m.p. 77-80°, R_{f}: 0.48 (solvent as in example 1a).

### EXAMPLE 33

### N¹,N⁴-bis(3-aminopropyl)-N¹,N⁴-bis [(2-hydroxy)-n-decyl]-1,4-diamino-trans-2-butene-trioxalate

A mixture of 1.65 g (0.002314 moles) of N¹,N⁴-bis(3-BOC-aminopropyl)-N¹,N⁴-bis[(2-hydroxy)-n-decyl]-1,4-diamino-trans-2-butene, 0.875 g (0.00694 moles) of oxalic acid dihydrate and 15 ml of water was boiled under reflux for 16 hours and then concentrated by evaporation under vacuum. After crystallisation of the residue from methanol, the title compound was obtained with a water content of 3.5%, m.p. 163-165° (decomp.).

The starting compound was produced as follows:

a) N¹,N⁴-bis(3-BOC-aminopropyl)-N¹,N⁴-bis[(2-hydroxy)-n-decyl]-1,4-diamino-trans-2-butene

The title compound was obtained in the form of an oil, analogously to example 11a, from 2 g (0.005 moles) of N¹,N⁴-bis(3-BOC-aminopropyl)-1,4-diamino-trans-2-butene (example 11b), 2.34 g (0.015 moles) of 1,2-decene oxide and 20 ml of ethanol (duration of reaction: 15 hours), using methylene chloride and a methylene chloride/methanol mixture (19:1) for the flash chromatography. R_{f}: 0.49 (solvent as in example 3a).

### EXAMPLE 34

### N¹,N¹²-bis(3-aminopropyl)-N¹,N¹²-bis[(2-hydroxy)-n-tetradecyl]-1,12-diamino-dodecane Tetraoxalate

A solution of 0.45 g (0.00357 moles) of oxalic acid dihydrate in 20 ml of acetonitrile was added whilst stirring to a solution of 0.66 g (0.000893 moles) of N¹,N¹²-bis(3-aminopropyl)-N¹,N¹²-bis[(2-hydroxy)-n-tetradecyl]-1,12-diamino-dodecane in 20 ml of methanol. The mixture was cooled to 0°, filtered, the residue washed with acetonitrile and dried under a high vacuum. The title compound was thus obtained, m.p. 87-89°.

The starting compound was produced as follows:

a) N¹,N¹²-bis(3-aminopropyl)-N¹,N¹²-bis[(2-hydroxy)-n-tetradecyl]-1,12-diamino-dodecane

0.81 g (0.0011 moles) of N¹,N¹²-bis(2-cyanoethyl)-N¹,N¹²-bis[(2-hydroxy)-n-tetradecyl]-1,12-diamino-dodecane were dissolved in 10 ml of an 11% solution of ammonia in ethanol, mixed with 0.4 g of Raney nickel and hydrogenated until the hydrogen uptake has ended. After filtering, concentrating the filtrate by evaporation under vacuum, and purifying the residue by flash chromatography on silica gel, using methylene chloride/methanol mixtures (40:1 or 10:1) and mixtures of methylene chloride/methanol/30% aqueous ammonia solution (90:10:0.5 or 40:10:1.5), the title compound was obtained in the form of an oil,

R_{f}: 0.34 (solvent as in example 1a), which gradually solidified into crystalline form.

b) N¹,N¹²-bis(2-cyanoethyl)-N¹,N¹²-bis[(2-hydroxy)-n-tetradecyl]-1,12-diamino-dodecane

11.99 g (0.048 moles) of 1,2-tetradecene oxide (85%) were added to a solution of 6.13 g (0.02 moles) of N¹,N¹²-bis(2-cyanoethyl)-1,12-diamino-dodecane [J. Med. Chem. 7, 710 (1964)] in 60 ml of ethanol. The reaction mixture was heated under reflux for 40 hours, a further 2.54 g (0.01016 moles) of tetradecene oxide (85%) were added, the reaction mixture was boiled under reflux for a further 6 hours, and then concentrated by evaporation under vacuum. Purification of the crude product was by flash chromatography on silica gel, using methylene chloride and methylene chloride/methanol mixtures (40:1 or 20:1). After concentrating the product-containing fractions by evaporation under vacuum and crystallizing the residue from acetonitrile, the title compound was obtained, m.p. 37-38°.

### EXAMPLE 35

### Preparation of Core Complexes of Plasmid Nucleic Acid with Substituted Aminoethanols and their Biological Activity

Preparation of core complexes of nucleic acid can be performed using substituted aminoethanols either with or without long chain hydrocarbon (aliphatic) substitutients.

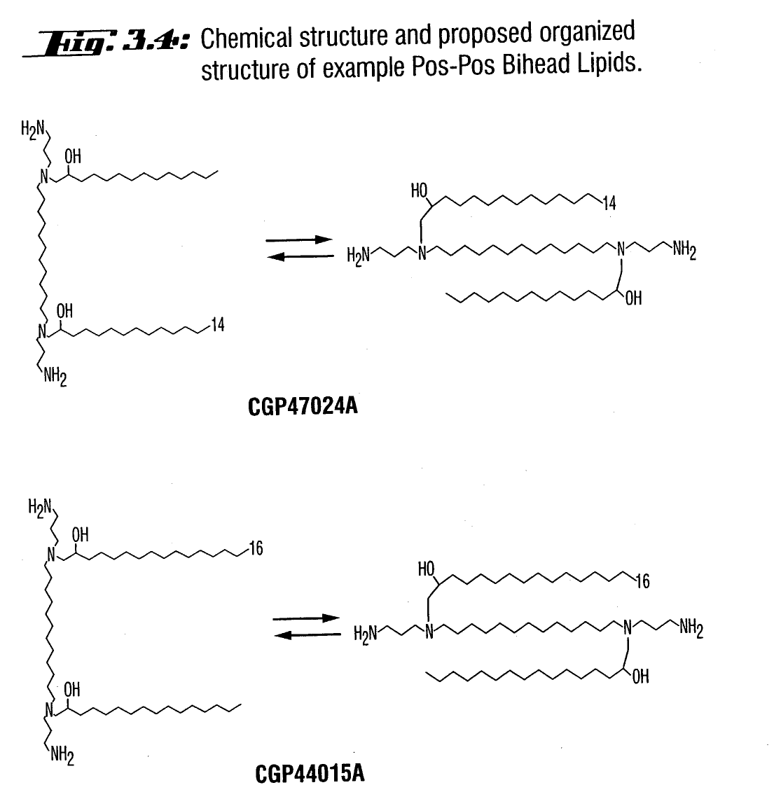

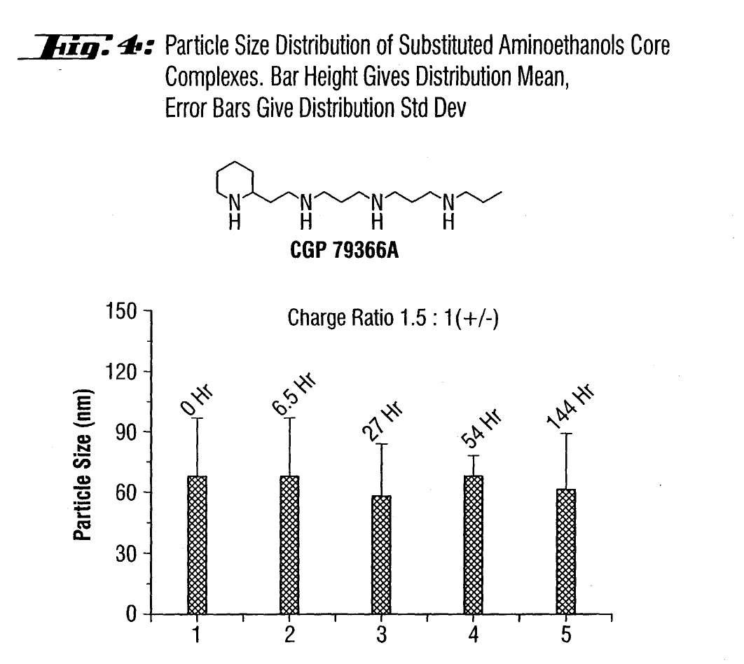

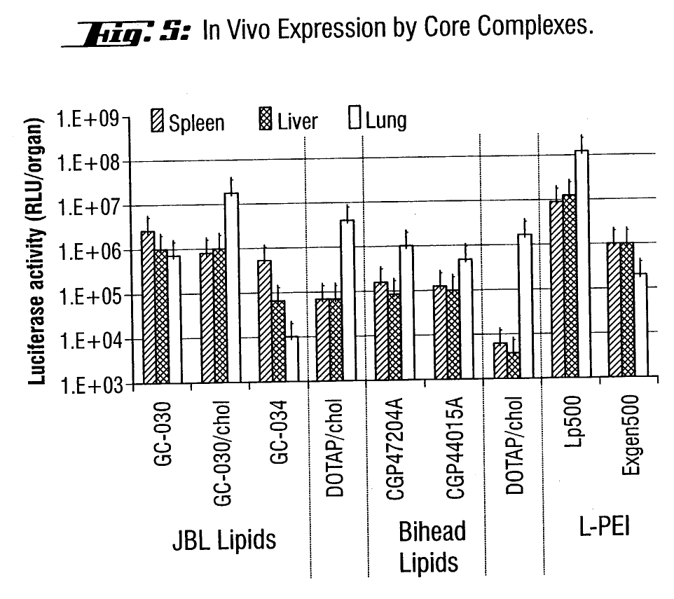

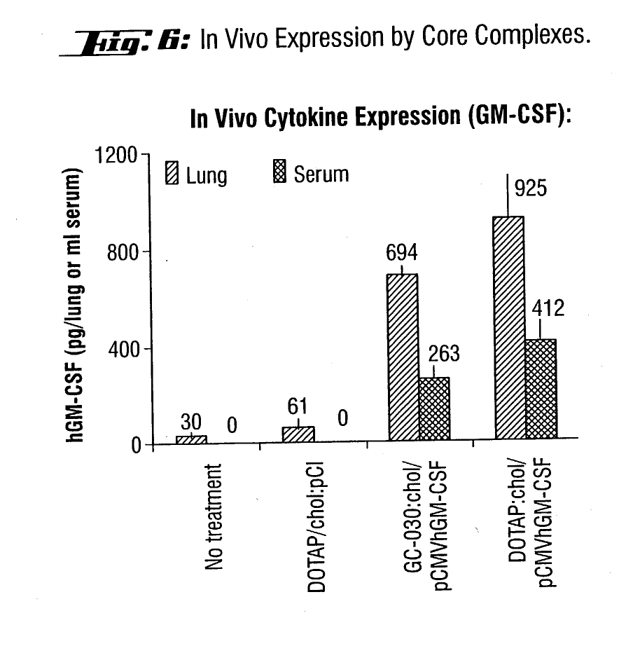

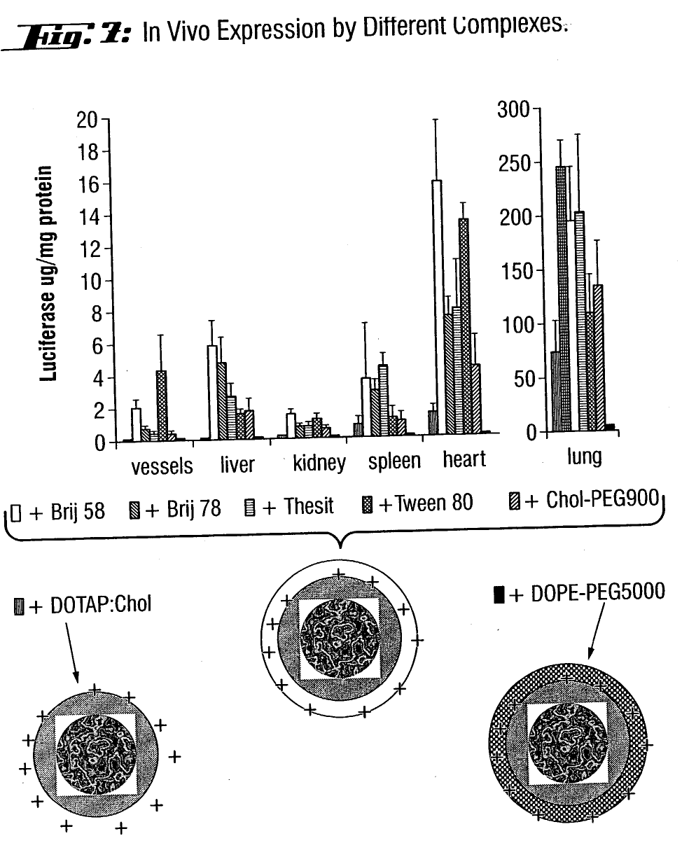

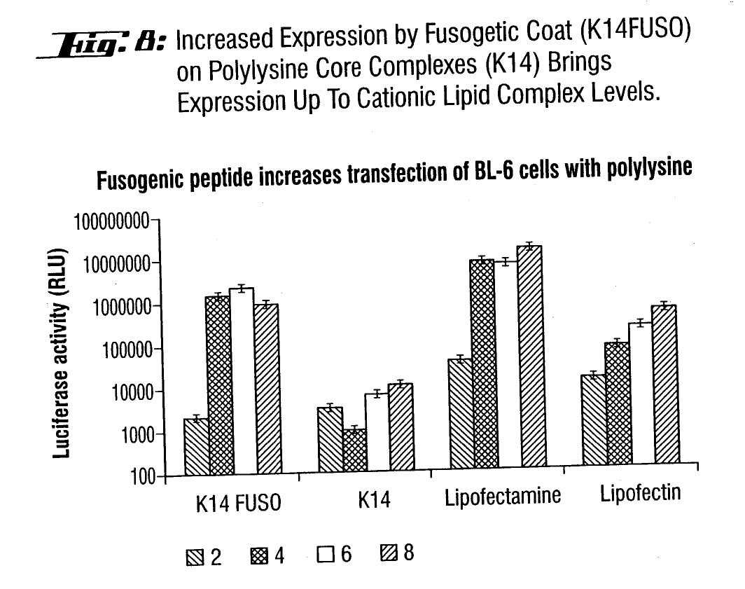

Substituted aminoethanols lacking long chain hydrocarbon (aliphatic) substitutients were used to compact plasmid DNA into a colloidal dispersion in water. The size and zeta potential of the colloidal dispersions prepared were determined at different charge ratios for added cation (amine) to anion (DNA phosphate) and are shown in Table 1 and FIG. 4. The colloidal dispersions prepared permit compaction of the DNA into core complexes that are suitable for the invention.

Substituted aminoethanols with long chain hydrocarbon (aliphatic) substitutients also were used to compact plasmid DNA into a colloidal dispersion in water. In some cases these core complexes alone are sufficient to provide gene delivery in cell culture or when administered to animals. This effect is illustrated in results below (Table 2 and 3).

TABLE 1
Particle size and Zeta Potential of Substituted Amino-Ethanol-DNA
complexes

| | | | | |
|---|---|---|---|---|
| Charge | | | | |
| Ratio | Particle Size | | Zeta | |
| (+/−) | (nm) | Std. Dev. | Potential | Std. Dev. |

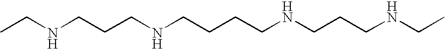

CGP 41660A

| | | | | |
|---|---|---|---|---|
| 0.5 | 136 | 66.4 | −40.9 | 8.04 |
| 1.0 | 62.6 | 19.3 | −8.72 | 7.62 |
| 1.5 | 93.4 | 33.9 | −7.67 | 12.1 |
| 2.0 | 573.5 | 254.5 | −7.08 | 4.24 |
| 3.0 | 987.4 | 454.7 | −5.03 | 4.01 |
| 4.0 | 4959 | 2297 | −4.87 | 6.8 |

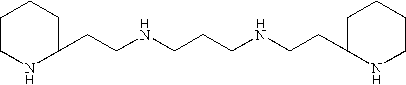

CGP 61670A

| | | | | |
|---|---|---|---|---|
| 0.5 | | | −17.6 | 8.79 |
| 1.0 | 77.6 | 18.8 | −15.4 | 4.99 |
| 1.5 | 192.4 | 48.3 | −9.1 | 4.72 |
| 2.0 | 293.3 | 119.5 | −8.71 | 5.37 |
| 3.0 | 3946.4 | 1849.9 | −6.01 | 2.52 |
| 4.0 | 4304.7 | 2018.9 | −5.52 | 7.79 |

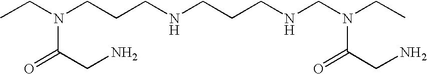

CGP 61750A

| | | | | |
|---|---|---|---|---|
| 0.5 | 176.7 | 81.7 | −47.3 | 12.8 |
| 1.0 | 77.6 | 42.9 | −30 | 6.75 |
| 1.5 | 90.3 | 31.3 | −25 | 11.5 |
| 2.0 | 206.8 | 33.6 | −17.1 | 12.4 |
| 3.0 | 1941.8 | 904.4 | −11.7 | 4.74 |
| 4.0 | 2526.8 | 1177.2 | −9.78 | 3.21 |

The gene delivery ability of substituted aminoethanols with long chain hydrocarbon (aliphatic) substitutients was studied by transfection of cultured cells and then in vivo by intravenous injection (Table 2 and 3). The substituted aminoethanols have two hydrophilic polar heads connected with one hydrophobic body, which was named bihead lipids. Bihead lipids are proposed to form a monolayer membrane.

Substituted aminoethanols (cationic compounds) were prepared as described in Examples 1-34. Their gene delivery ability was studied in vivo by intravenous injection (Table 1 and 2) using a standard method. The preparations were administered via tail vein injection to mice and gene expression determined after 5 hours. Female CD-1 mice, 13-15 g, were purchased from Charles River Inc. Forty microgram of pCILuc complexed with GC lipids or GC lipid:Chol dispersion as indicated weight ratios. After 5 h, mice were sacrificed and organs were collected. Organs were homogenized in 0.5 ml of lysis buffer and 20 μl of supernatant was used for luciferase assay. Luciferase activity was represented as a mean of relative light unit (RLU) of four mice. The lipids were either used alone or combined with cholesterol and complexed with a luciferase reporter gene plasmid by a standard procedure at a range of weight and charge ratios. For the in vivo screen, 40 μg of pCILuc was complexed with the formulation and injected into the mice.

The relationship of structure and gene delivery function also was studied. The number and the length of fat acid chains were found to impact their gene delivery ability. If the lipids had only one chain, transfection activity was not observed, regardless of the length of the acid chains. If the length of two chains was shorter than C14, transfection activity also was not observed. If the lipid had one short chain (<C14) and one long chain (>C14), it could not deliver genes. However, with longer chains such C14 and C 16, the lipids showed transfection activity not only in vitro as also in vivo. The in vitro transfection activity was even higher than that of commercially available lipid preparations, such as Lipofectamine 7.

When the length of carbon chain between two ammonium groups in the hydrophilic polar head increased from C4 to C12, the conformation of lipids in water may change from that of a typical lipid with one head to a form with two heads at each end of the molecule. Accordingly, such lipids are referred to herein as bihead lipids and this is shown in FIG. 3.

Substituted aminoethanols CGP44015 and CGP47204, chemical structures shown in FIG. 3.4, disperse in water to form very small homogenous micelles with a diameter around 10-20 nm. They bind to plasmid DNA forming core complexes with a particle size dependent on the charge ratio of cationic compound to DNA. In this aspect they are representative of the substituted aminoethanol class of compounds giving small, relatively homogenous, and stable complexes with nucleic acids as illustrated with a different compound in FIG. 4. When the charge ratio is more than 1, the particles are homogenous with diameter less than 100 nm. Their transfection activity increases with increasing charge ratio to 4. The optimal charge ratio in vitro is 4. The transfection activity decreased with further increase in charge ratio. The in vivo transfection activity and charge ratio relationship was similar to that in vitro but the optimal charge ratio is 4-10 (Table 1 and 2). Overall, the compounds with the same charged head groups showed good complexation with plasmid DNA and gene delivery in vitro as well as in vivo.

The substituted aminoethanols tested here appear to have two hydrophilic polar heads connected by one hydrophobic body (FIG. 3) and are referred to as bihead lipids. Since two hydrophilic heads at either side could face an aqueous solution, these compounds could form a monolayer in water instead of a bilayer formed by lipids with one head group (FIG. 3.1).

These results show good gene transfer ability. Among the preferred lipids, CGP44015A and CGP47204A form core complexes that exhibit expression in vivo. CGP44015 and CGP47204 have the same positive charges in both heads. The bihead lipids show high gene transfer ability in vitro as well as in vivo.

TABLE 2

| | | Luciferase | |
|---|---|---|---|
| | | activity | |
| | Charge | Ave. RLU × 10000/well | |
| Compound No. | Ratio(+/−) | FBS(−) | FBS(+) |
| CGP42395A | 0.5 | 0 | 0 |
| 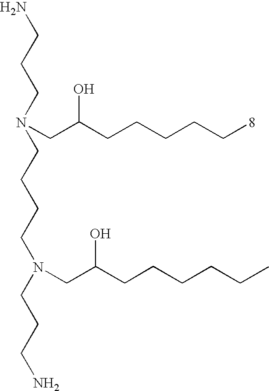 | 1 2 4 6 8 | 0 1 0 0 0 | 0 0 0 0 0 |
| CGP41062A | 0.5 | 0 | 0 |
| 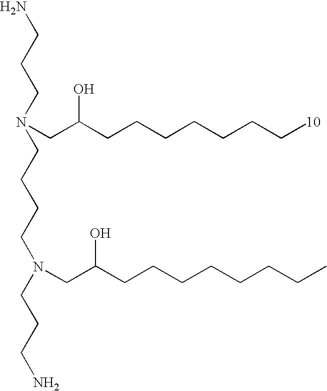 | 1 2 4 6 8 | 0 0 0 3 17 | 0 0 0 2 15 |
| CGP42396A | 0.5 | 0 | 0 |
| 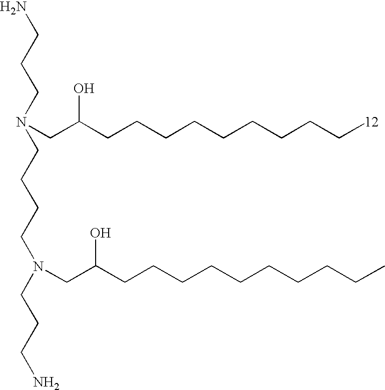 | 1 2 4 6 8 | 37 0 0 4 27 | 36 0 0 2 13 |
| CGP42397A | 0.5 | 1 | 1 |
| 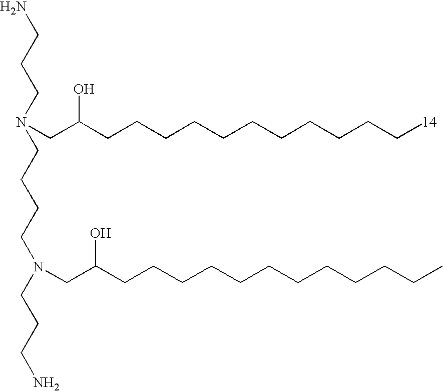 | 1 2 4 6 8 | 146 730 927 905 2,137 | 67 538 718 754 1,557 |
| CGP40337A | 0.5 | 21 | 18 |
| 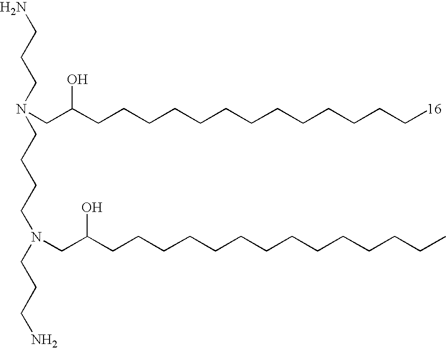 | 1 2 4 6 8 | 479 4,118 21,488 17,626 18,395 | 364 4,156 22,149 19,000 19,832 |
| Lipofectamine | ? | 3,208 | 2,548 |
| NVP-AAV120-AH-1 | 0.5 | 2 | 7 |
| 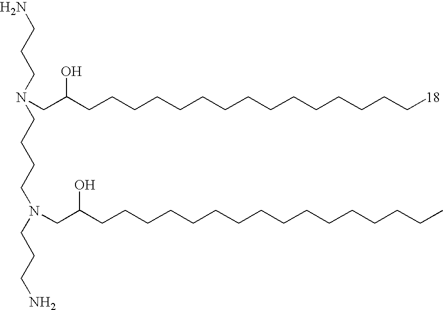 | 1 2 4 6 8 | 1 1 0 1 3 | 4 2 1 1 8 |
| CGP47204A | 0.5 | 33 | 11 |
| 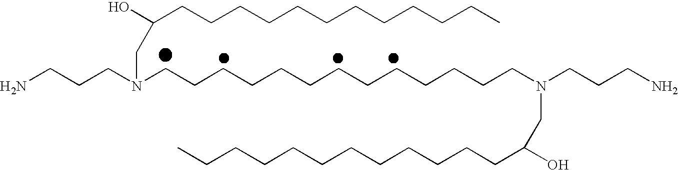 | 1 2 4 6 8 | 737 8,386 15,009 20,091 17,740 | 821 8,079 13,088 22,694 21,644 |
| CGP44015A | 0.5 | 159 | 130 |
| 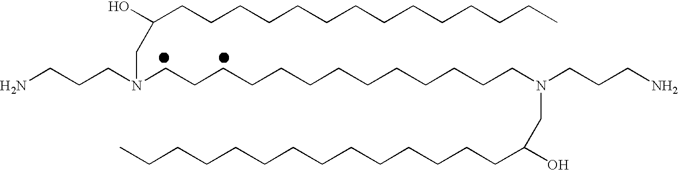 | 1 2 4 6 8 | 3,252 17,063 24,484 24,089 24,150 | 3,373 17,315 25,732 27,844 27,298 |
| CGP46091A | 0.5 | 171 | 191 |
| 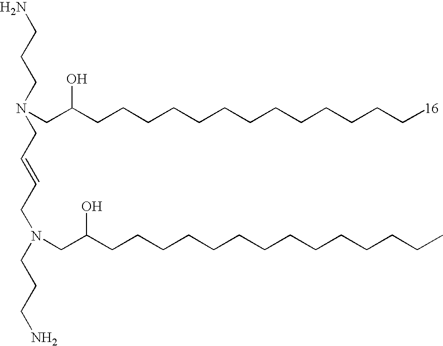 | 1 2 4 6 8 | 701 4,893 7,090 11,037 10,146 | 817 5,233 9,074 13,813 12,615 |
| CGP44207A | 0.5 | 12 | 12 |
| 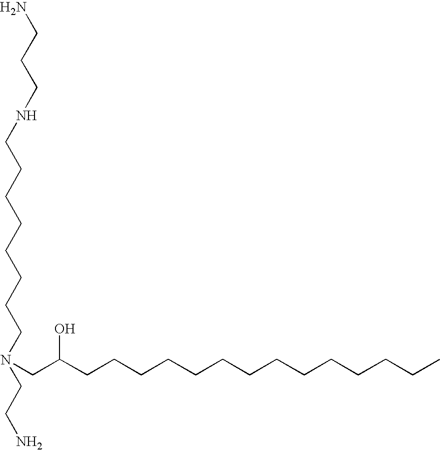 | 1 2 4 6 8 | 12 11 15 28 22 17 | 12 9 12 22 15 13 |
| CGP40200A | 0.5 | 12 | 12 |
| 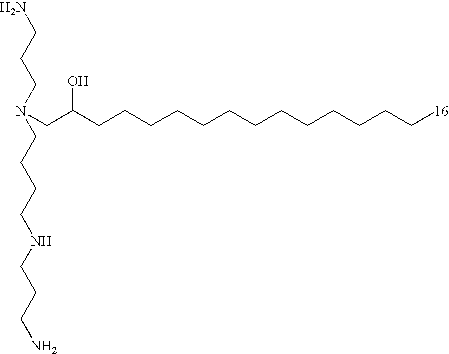 | 1 2 4 6 8 | 12 7 7 13 9 9 | 12 7 7 16 7 10 |

TABLE 3

| | | RLU/20 ul lysate | | | | | SEM | | | | |
|---|---|---|---|---|---|---|---|---|---|---|---|
| Compound No | CR | spleen | liver | kidney | heart | lung | spleen | liver | kidney | heart | lung |
| 42395A | 0.5 | 68 | 67 | 64 | 80 | 294 | 2 | 1 | 1 | 10 | 138 |
| | 1 | 60 | 69 | 71 | 62 | 63 | 1 | 2 | 9 | 2 | 3 |
| | 4 | 63 | 63 | 63 | 63 | 88 | 2 | 2 | 3 | 2 | 25 |
| | 10 | N | N | N | N | N | | | | | |
| | 20 | N | N | N | N | N | | | | | |
| 41062A | 0.5 | 36 | 29 | 34 | 39 | 175 | 4 | 1 | 2 | 4 | 39 |
| | 1 | 34 | 29 | 30 | 49 | 53 | 1 | 1 | 1 | 10 | 13 |
| | 4 | 60 | 31 | 41 | 44 | 93 | 29 | 2 | 7 | 8 | 28 |
| | 10 | N | N | N | N | N | | | | | |
| | 20 | N | N | N | N | N | | | | | |
| 42396A | 0.5 | 67 | 59 | 50 | 52 | 82 | 5 | 3 | 1 | 1 | 12 |
| | 1 | 54 | 53 | 52 | 52 | 65 | 2 | 2 | 2 | 1 | 5 |
| | 4 | 50 | 53 | 54 | 52 | 58 | 2 | 1 | 1 | 1 | 2 |
| | 10 | 54 | 51 | 53 | 54 | 51 | 2 | 2 | 1 | 1 | 1 |
| | 20 | 60 | 57 | 59 | 58 | 62 | 1 | 1 | 2 | 1 | 3 |
| 42397A | 0.5 | 55 | 60 | 56 | 63 | 77 | 1 | 2 | 0 | 9 | 9 |
| | 1 | 59 | 59 | 57 | 61 | 57 | 3 | 1 | 2 | 1 | 1 |
| | 4 | 67 | 142 | 57 | 56 | 62 | 7 | 23 | 1 | 1 | 5 |
| | 10 | 140 | 2,026 | 69 | 69 | 122 | 39 | 1,017 | 2 | 2 | 39 |
| | 20 | 1,183 | 5,429 | 88 | 64 | 352 | 865 | 2,077 | 16 | 4 | 176 |
| 40337A | 0.5 | 69 | 72 | 65 | 77 | 92 | 1 | 3 | 1 | 7 | 8 |
| | 1 | 72 | 70 | 57 | 60 | 68 | 8 | 3 | 2 | 1 | 3 |
| | 4 | 343 | 4,792 | 64 | 59 | 87 | 195 | 1,134 | 2 | 5 | 11 |
| | 10 | 270 | 17,541 | 92 | 63 | 112 | 72 | 9,150 | 16 | 2 | 16 |
| | 20 | N | N | N | N | N | | | | | |
| DOTAP/chol | 4 | 2,391 | 2,212 | 1,081 | 5,174 | 461,133 | 607 | 446 | 59 | 604 | 88,678 |
| 38634B | 0.5 | 50 | 49 | 47 | 56 | 75 | 2 | 1 | 2 | 2 | 8 |
| | 1 | 45 | 44 | 58 | 46 | 51 | 1 | 1 | 13 | 2 | 3 |
| | 4 | 47 | 43 | 45 | 42 | 56 | 3 | 1 | 1 | 1 | 6 |
| | 10 | 43 | 40 | 39 | 40 | 61 | 2 | 1 | 1 | 1 | 11 |
| | 20 | N | N | N | N | N | | | | | |
| 40200A | 0.5 | 44 | 44 | 40 | 49 | 57 | 1 | 3 | 1 | 2 | 5 |
| | 1 | 42 | 152 | 44 | 40 | 48 | 1 | 78 | 1 | 1 | 6 |
| | 4 | 68 | 69 | 66 | 68 | 73 | 0 | 1 | 1 | 2 | 7 |
| | 10 | 60 | 348 | 102 | 63 | 69 | 1 | 150 | 32 | 2 | 8 |
| | 20 | 64 | 74 | 67 | 73 | 71 | N | N | N | N | N |
| 45247A | 0.5 | 66 | 75 | 65 | 68 | 65 | 1 | 10 | 2 | 2 | 3 |
| | 1 | 62 | 60 | 58 | 64 | 60 | 1 | 2 | 1 | 1 | 1 |
| | 4 | 68 | 103 | 67 | 62 | 81 | 1 | 32 | 3 | 2 | 3 |
| | 10 | 66 | 65 | 64 | 79 | 77 | 3 | 1 | 1 | 9 | 4 |
| | 20 | 65 | 73 | 63 | 68 | 63 | 1 | 10 | 3 | 2 | 3 |
| 43656A | 0.5 | 71 | 382 | 82 | 74 | 132 | 1 | 259 | 9 | 7 | 50 |
| | 1 | 71 | 71 | 61 | 94 | 114 | 5 | 5 | 1 | 28 | 21 |
| | 4 | 68 | 71 | 70 | 72 | 95 | 1 | 1 | 1 | 1 | 4 |
| | 10 | N | N | N | N | N | | | | | |
| | 20 | N | N | N | N | N | | | | | |
| DOTAP/chol | 4 | 2,962 | 3,665 | 739 | 4,146 | 253,914 | 493 | 1,510 | 349 | 828 | 77,241 |
| CGP047204A | 0.5 | 80 | 45 | 39 | 38 | 44 | | | | | |
| | 1 | 467 | 53 | 38 | 41 | 51 | | | | | |
| | 4 | 5,594 | 4,583 | 386 | 129 | 5,009 | | | | | |
| | 10 | 6,378 | 3,270 | 156 | 983 | 38,115 | | | | | |
| | 20 | 779 | 429 | 113 | 96 | 3,258 | | | | | |
| CGP044015A | 0.5 | 80 | 86 | 72 | 74 | 82 | | | | | |
| | 1 | 168 | 219 | 71 | 73 | 95 | | | | | |
| | 4 | 4,778 | 3,817 | 528 | 379 | 19,717 | | | | | |
| | 10 | 4,108 | 1,283 | 281 | 103 | 4,470 | | | | | |
| | 20 | 69 | 243 | 70 | 75 | 84 | | | | | |
| DOTAP/chol | 4 | 239 | 144 | 104 | 468 | 63,609 | | | | | |

### EXAMPLE 36

### Preparation of Core Complexes of Plasmid Nucleic Acid with Cationic Lipids

Cationic lipids GC-001, GC-003, GC-016, GC-021, GC-025, GC-026, GC-029, GC-030, GC-033, GC-034, GC-035, GC-38, GC-039, and GC-071 were purchased from Promega Biosciences, San Luis Obispo, Calif. [formerly JBL Scientific, Inc/Genta]. Other materials and methods were performed as described in Example 35. The measurement of luciferase expression in selected organs is summaried in Table 3.

All compounds were evaluated for in vivo activity. Two critical factors were examined, formulation with or without cholesterol and the ratio of cationic lipid to DNA. Cholesterol was tested at 1:1 mole ratio of lipid:chol. The studies were performed with a dose of 40 μg of pCILuc complexed with cationic lipid or lipid: Chol (1:1 mole ratio) injected i.v. into CD-1 mice and then luciferase activity in different organs determined 5 h later. The first evaluation included all of 14 GC-lipids at weight ratios of 2 and 10 (GC lipid to DNA). It was performed by four separated experiments. Each time cationic liposome DOTAP:Chol was used as a standard control. Results were shown in Table 4. Many GC lipid formulations showed luciferase activity more than 2000 RLU/20 μl lysate in spleen and liver.

Measurements were repeated with lipids GC-030, GC-034 and GC-029 at wider weight ratios than the first experiment. The transfection procedure was the same as that for results shown in Table 3. Luciferase activity is represented as a mean of relative light unit (RLU) of four mice. WR means weight ratio of GC lipids to DNA. The results are shown in Table 5. The transfection activity was represented by luciferase activity RLU/organ. GC-030 showed high transfection activity at weight ratio 20. The transfection activity increased with the increased weight ratio (GC lipids to DNA). Inclusion of cholesterol can change the biodistribution of gene expression in the different organs examined. For example, GC-030 alone resulted in high luciferase activity in spleen and GC-030:Chol resulted in high luciferase activity in lung. However this function of cholesterol was not seen with GC-034. GC-030 showed high luciferase activity in spleen at weight ratio 20, in fact 36 fold higher than that of the DOTAP:Chol standard. Likewise, GC-030:Chol showed high luciferase activity in lung, about 5 fold higher than that of DOTAP:Chol. These results show that GC lipids form good core complexes for gene delivery vectors.

TABLE 4
Evaluation of GC lipids in mice via IV injection.

| | | RLU/20 μl lysate | | | | |
|---|---|---|---|---|---|---|
| | WR | spleen | liver | kidney | heart | lung |
| GC-001 | 2 | 38 | 35 | 39 | 36 | 51 |
| GC-001 | 10 | 329 | 38 | 36 | 39 | 101 |
| GC-001/chol | 2 | 46 | 39 | 33 | 36 | 57 |
| GC-001/chol | 10 | 40 | 34 | 34 | 34 | 40 |
| GC-003 | 2 | 36 | 33 | 38 | 37 | 37 |
| GC-003 | 10 | 65 | 73 | 40 | 39 | 69 |
| GC-003/chol | 2 | 323 | 39 | 36 | 34 | 90 |
| GC-003/chol | 10 | 46 | 37 | 34 | 40 | 54 |
| GC-021 | 2 | 43 | 36 | 34 | 34 | 54 |
| GC-021 | 10 | 37 | 32 | 35 | 39 | 40 |
| GC-021/chol | 2 | 78 | 81 | 46 | 44 | 64 |
| GC-021/chol | 10 | 62 | 60 | 57 | 58 | 120 |
| DOTAP/chol | 8.5 | 2,756 | 2,721 | 360 | 2,134 | 150,682 |
| | | RLU/20 ul lysate | | | | |
| | WR | spleen | liver | kidney | heart | lung |
| GC-016 | 2 | 40 | 38 | 41 | 44 | 45 |
| GC-016 | 10 | 230 | 95 | 40 | 40 | 67 |
| GC-016/chol | 2 | 38 | 39 | 40 | 45 | 44 |
| GC-016/chol | 10 | 111 | 1,007 | 52 | 43 | 449 |
| GC-026 | 2 | 40 | 39 | 41 | 42 | 40 |
| GC-026 | 10 | 0 | 0 | 0 | 0 | 0 |
| GC-026/chol | 2 | 47 | 46 | 50 | 54 | 49 |
| GC-026/chol | 10 | 57 | 52 | 88 | 1,315 | 50 |
| GC-030 | 2 | 3,692 | 70 | 51 | 48 | 88 |
| GC-030 | 10 | 5,305 | 1,093 | 105 | 67 | 1,396 |
| GC-030/chol | 2 | 61 | 51 | 51 | 47 | 48 |
| GC-030/chol | 10 | 1,330 | 3,065 | 246 | 60 | 574 |
| GC-039 | 2 | 288 | 48 | 45 | 50 | 52 |
| GC-039 | 10 | 845 | 212 | 54 | 45 | 753 |
| GC-039/chol | 2 | 49 | 47 | 48 | 46 | 49 |
| GC-039/chol | 10 | 84 | 376 | 4,503 | 78 | 173 |
| DOTAP/chol | 8.5 | 549 | 153 | 93 | 9,725 | 8,163 |
| GC-025 | 2 | 46 | 38 | 25 | 33 | 40 |
| GC-025 | 10 | 32 | 27 | 28 | 27 | 30 |
| GC-025/chol | 2 | 26 | 26 | 28 | 27 | 26 |
| GC-025/chol | 10 | 65 | 66 | 26 | 28 | 33 |
| GC-033 | 2 | 27 | 29 | 27 | 31 | 36 |
| GC-033 | 10 | 50 | 46 | 41 | 40 | 42 |
| GC-033/chol | 2 | 45 | 46 | 49 | 46 | 45 |
| GC-033/chol | 10 | 50 | 86 | 47 | 46 | 47 |
| GC-035 | 2 | 70 | 57 | 46 | 54 | 57 |
| GC-035 | 10 | 54 | 50 | 54 | 51 | 54 |
| GC-035/chol | 2 | 82 | 72 | 47 | 52 | 51 |
| GC-035/chol | 10 | 91 | 84 | 46 | 43 | 329 |
| DOTAP/chol | 8.5 | 1011 | 331 | 78 | 1412 | 45457 |
| GC-029 | 2 | 31 | 31 | 34 | 42 | 41 |
| GC-029 | 10 | 196 | 65 | 42 | 34 | 72 |
| GC-029/chol | 2 | 31 | 29 | 31 | 39 | 34 |
| GC-029/chol | 10 | 1,512 | 104 | 33 | 33 | 53 |
| GC-034 | 2 | 7,769 | 480 | 36 | 45 | 137 |
| GC-034 | 10 | 2,386 | 597 | 53 | 103 | 90 |
| GC-034/chol | 2 | 63 | 80 | 59 | 62 | 63 |
| GC-034/chol | 10 | 1,645 | 1,597 | 58 | 70 | 267 |
| GC-038 | 2 | 61 | 59 | 60 | 101 | 142 |
| GC-038 | 10 | 160 | 93 | 58 | 57 | 63 |
| GC-038/chol | 2 | 56 | 55 | 75 | 69 | 268 |
| GC-038/chol | 10 | 783 | 132 | 130 | 140 | 1,210 |
| GC-071 | 2 | 61 | 59 | 58 | 60 | 56 |
| GC-071 | 10 | 286 | 531 | 67 | 61 | 73 |
| GC-071/chol | 2 | 95 | 60 | 59 | 60 | 63 |
| GC-071/chol | 10 | 263 | 476 | 64 | 60 | 187 |
| DOTAP/chol | 8.5 | 909 | 1,084 | 281 | 603 | 101,852 |

TABLE 5
Evaluation of selected GC lipids in mice.

| Liposome | WR | spleen | liver | kidney | heart | lung |
|---|---|---|---|---|---|---|
| GC-030 | 1 | 23,308 | 3,642 | 1,392 | 1,675 | 3,308 |
| GC-030 | 2 | 73,442 | 3,417 | 1,458 | 1,367 | 1,600 |
| GC-030 | 6 | 38,058 | 1,550 | 792 | 817 | 8,808 |
| GC-030 | 10 | 446,650 | 114,425 | 1,367 | 3,450 | 35,117 |
| GC-030 | 20 | 2,479,217 | 1,003,125 | 17,583 | 4,783 | 689,475 |
| GC-030/chol | 2 | 202,158 | 5,283 | 1,167 | 1,058 | 3,983 |
| GC-030/chol | 10 | 593,158 | 141,383 | 5,808 | 7,058 | 1,965,650 |
| GC-030/chol | 15 | 581,875 | 452,575 | 10,892 | 54,642 | 4,353,292 |
| GC-030/chol | 20 | 820,250 | 894,608 | 38,233 | 428,575 | 17,411,233 |
| GC-034 | 0.5 | 8,750 | 1,792 | 1,300 | 1,425 | 3,567 |
| GC-034 | 1 | 10,458 | 2,758 | 1,283 | 1,333 | 4,492 |
| GC-034 | 2 | 29,167 | 3,317 | 1,175 | 1,158 | 2,367 |
| GC-034 | 6 | 449,583 | 10,533 | 1,567 | 1,467 | 6,233 |
| GC-034 | 10 | 505,975 | 63,942 | 1,750 | 1,642 | 9,775 |
| GC-034/chol | 2 | 7,392 | 4,017 | 1,500 | 1,425 | 2,575 |
| GC-034/chol | 10 | 58,933 | 4,975 | 1,558 | 1,483 | 13,592 |
| GC-034/chol | 15 | 39,208 | 4,775 | 1,383 | 1,450 | 5,958 |
| GC-034/chol | 20 | 37,542 | 7,475 | 1,492 | 1,317 | 9,892 |
| DOTAP/chol | 8.5 | 68,908 | 68,025 | 9,000 | 53,342 | 3,767,050 |
| Liposome | WR | spleen | liver | kidney | Heart | lung |
| GC-029 | 2 | 908 | 825 | 1,000 | 933 | 967 |
| GC-029 | 10 | 1,992 | 1,408 | 808 | 858 | 1,017 |
| GC-029/chol | 2 | 842 | 817 | 842 | 908 | 875 |
| GC-029/chol | 6 | 942 | 942 | 908 | 950 | 1,125 |
| GC-029/chol | 10 | 867 | 958 | 933 | 950 | 3,308 |
| GC-029/chol | 18 | 4,250 | 3,875 | 950 | 858 | 2,392 |
| DOTAP/chol | 8.5 | 9,267 | 11,350 | 3,650 | 8,042 | 1,114,275 |

### EXAMPLE 37

### Preparation of Linear PEI

Linear PEI of MW of 22 kDa was prepared from polyethyloxazoline polymer (PEOZ) by acid hydrolysis to the polyamine. The PEOZ was prepared by polymerization using methyl tosylate and 500 equivalents of 2-ethyl-2-oxazoline following essentially the same previously reported procedure by Zalipsky et al. J. Pharm. Sci.; 85: 133-137 (1996). It was necessary to use 2-ethyl-2-oxazoline instead of 2-methyl-2-oxazoline as the latter precipitated at MW 16,200 in acetonitrile. Also longer reaction times were needed.

Preparation of Poly(2-ethyl-2-oxazoline) of MW 49,500 kDa.

Polymerization reaction was conducted in a screw-cap tube that was dried under vacuo while heated prior to use. The tube was charged with 5.05 ml of 2-ethyl-2-oxazoline that was freshly distilled over KOH and 5 ml of dry acetonitrile. 491 mg of freshly distilled methyl tosylate was dissolved in 10.55 ml of dry acetonitrile and 0.4 ml of this solution was transferred to the tube containing the monomer. After this transfer the tube was purged with argon, sealed and left stirring in an oil bath at 80° C. for 112 h. After cooling to room temperature 2 ml of a methanolic-solution of KOH (0.5M) was added to the polymerization mixture followed by stirring at 25° C. for 5 h. 0.2 ml of glacial acetic acid was added and the mixture concentrated to solid, redissolved in 50 ml of water and placed in 3500 molecular weight cutoff Spectral/Por dialysis membranes (Spectrum, Los Angeles, Calif.). Dialysis was against 50 mM NaCl (1×4L) and water (3×4L). The content of the dialysis bags were lyophilized and further dried under vacuo to give 4.51 g of white solid (91%). Mass spectral analyses (MALDI-TOF) showed cluster at m/z 45,000-65,000 and centered at m/z 52,395 (expected m/z 49,500).

¹H NMR (400 MHz CDCl₃) δ 1.11-1.12 (m, CH₃CH₂C═O), 2.31-2.41 (m, CH₃CH₂C═O), 3.46 (m, CH₂N)

¹³C NMR (100 MHz CDCl₃) δ 9.2 (bs, CH₃CH₂C═O), 25.82 (s, CH₃CH₂C═O), 43.54-47.27 (m, CH₂N), 173.79-174.40 (m, C═O)

Preparation of Linear Polyethylenimine of MW 22 kDa.

The acid hydrolsis was conducted in a screw-cap tube. The tube was charged with 0.1 g of Poly(2-ethyl-2-oxazoline) of MW 49,500 kDa and 10 ml of 3.3 M aqueous HCl. The solution was degassed, purged with argon, sealed and left stirring in an oil bath at 100° C. for 65 h. Higher acid concentrations lead to precipitation during hydrolysis at 100° C. After cooling the mixture is concentrated to a solid, redissolved in water and again concentrated to a solid. Redissolved in 1 ml of water and pH was adjusted to 12-13 upon the addition of 2.5 M aqueous NaOH. The precipitate of linear polyethylenimine was collected by centrifugation and further washed with water (2×1 ml) to give 43 mg of white solid (100%).

¹H NMR (360 MHz, CD₃OD) δ 2.73 (br, CH₂N)

### EXAMPLE 38

Streams of salmon sperm DNA, at a concentration of 50 μg/ml and of polyethyleneimine were fed into an HPLC static mixer which included three 50 μl cartridges in tandem. In the making of each preparation of particles, each stream was fed into the mixer at the same flow rate, and such flow rate was maintained as the resulting combined stream of DNA and polymer flowed through the cartridges. Flow rates were from 250 μl/min. to 5,000 μl/min. The particle sizes for each preparation made at a given flow rate are given in Table 6 below.

TABLE 6
Particle Size

| Flow Rate | Unimodal | Std. dev. | % std. | SDP | Std. dev. | % std. |
|---|---|---|---|---|---|---|
| (μl/min.) | mean | unimodal | dev. | mean | SDP | dev. |
| 250 | 193.9 | 55.7 | 29 | 208.5 | 53.1 | 25 |
| 500 | 166 | 52.5 | 32 | 193.9 | 44.4 | 23 |
| 1,000 | 144.2 | 47.7 | 33 | 184.6 | 108.6 | 59 |
| 1,500 | 132.3 | 42 | 32 | 163.9 | 64.4 | 39 |
| 2,000 | 121.5 | 41.4 | 34 | 131.1 | 32.9 | 25 |
| 2,500 | 112.4 | 37.6 | 33 | 125.7 | 32.1 | 26 |
| 3,000 | 107.1 | 35 | 33 | 153.6 | 124.2 | 81 |
| 4,000 | 110.8 | 35.7 | 32 | 119.4 | 48 | 40 |
| 5,000 | 129.5 | 43.5 | 34 | 131.2 | 34.4 | 26 |

### EXAMPLE 39

The procedure of Example 38 was repeated, except that the streams of DNA and polyethyleneimine were fed into an HPLC mixer containing three 150 μl cartridges in tandem and flow rates varied from 500 μl/min. to 7,000 μl/min. The particle sizes for each preparation made at a given flow rate are given in Table 7 below.

TABLE 7
Particle Size

| Flow Rate | Unimodal | Std. dev. | % std. | SDP | Std. dev. | % std. |
|---|---|---|---|---|---|---|
| (μl/min.) | mean | unimodal | dev. | mean | SDP | dev. |
| 500 | 200.6 | 59.6 | 30 | 218.9 | 57.8 | 26 |
| 1,000 | 165.4 | 45.2 | 27 | 181 | 42.3 | 23 |
| 1,500 | 146.2 | 40.4 | 28 | 165.5 | 53.4 | 32 |
| 2,000 | 134.7 | 41.2 | 31 | 135.9 | 40 | 29 |
| 2,500 | 131.4 | 43 | 33 | 138.2 | 33.8 | 24 |
| 3,000 | 130.6 | 41.4 | 32 | 136.4 | 45.7 | 34 |
| 5,000 | 126.4 | 42.2 | 33 | 138.3 | 38.3 | 28 |
| 6,000 | 134 | 41.7 | 31 | 173.2 | 67.4 | 39 |
| 7,000 | 140.9 | 43.4 | 31 | 141.2 | 31.5 | 22 |

The results of Examples 38 and 39 show that particle size can be adjusted by changing the size of the mixing cartridges and by changing the flow rate. Thus, one can choose conditions which will provide particles of a desired size and homogeneity.

### EXAMPLE 40

The procedure of Example 38 was repeated, except that sodium chloride in varying concentration was added to the DNA and polymer after the mixing of the DNA and polymer. The mean particle sizes for each preparation made at a given concentration of salt are given in Table 8 below.

TABLE 8
Particle Size

| NaCl | | | | | Std. | |
|---|---|---|---|---|---|---|
| Concentration | Unimodal | Std. dev. | % std. | SDP | dev. | % std. |
| (mM) | mean | unimodal | dev. | mean | SDP | dev. |
| 0 | 108.3 | 27 | 25 | 129.2 | 71.1 | 55 |
| 5 | 202.8 | 46.4 | 23 | 213.5 | 44.1 | 21 |
| 20 | 200.4 | 32.8 | 16 | 206.6 | 16.2 | 8 |
| 100 | 372.9 | 85.9 | 23 | 360.1 | 28.3 | 8 |

The above results show that particle size can be controlled with the addition of salt, and that such particles remain uniform in size.

### EXAMPLE 41

The procedure of Example 38 was repeated, except that the DNA concentration was 100 μg/ml, and flow rates were varied from 500 μl/min. to 4,000 μl/min. The particle sizes for each preparation made at a given flow rate are given in Table 9 below.

TABLE 9
Particle Size

| Flow Rate | Unimodal | Std. dev. | % std. | SDP | Std. dev. | % std. |
|---|---|---|---|---|---|---|
| (μl/min.) | mean | unimodal | Dev. | mean | SDP | dev. |
| 500 | 215.2 | 14.9 | 7 | 213 | 20 | 9 |
| 1,000 | 191.8 | 38.8 | 20 | 196.4 | 25.7 | 13 |
| 1,500 | 199 | 47.8 | 24 | 198.6 | 23 | 12 |
| 2,000 | 163.6 | 12.6 | 8 | 163.9 | 17.2 | 10 |
| 2,500 | 172 | 29 | 17 | 174.4 | 27.1 | 16 |
| 3,000 | 192.7 | 20.5 | 11 | 198 | 23.6 | 12 |
| 4,000 | 166.7 | 14.7 | 9 | 162.7 | 14.9 | 9 |

The above results, when compared with those of Example 38, show that particle size can be changed by changing the concentration of DNA.

### EXAMPLE 42

The procedure of Example 38 was repeated, except that the mixer contained one 250 μl cartridge, and Tween 80 detergent in an amount of 0.25% by volume was added to the DNA stream prior to mixing with the polyethyleneimine stream and flow rates were varied from 210 μl/min. to 8,400 μl/min. for the DNA and Tween 80 stream. When the DNA and Tween 80 stream and the polymer stream were fed initially into the mixer, the flow rate of the DNA and Tween 80 stream was 1.4 times that of the polymer stream. When the combined stream of DNA and Tween 80 and polymer traveled through the cartridge, the flow rate of the combined stream was the average of the initial flow rates of the DNA and Tween 80 stream and the polymer stream. For example, if the DNA and Tween 80 stream had an initial flow rate of 4,900 μl/min. and the polymer stream had a flow rate of 3,500 μl/min., the flow rate of the combined stream through the cartridge was 4,200 μl/min. The particle sizes for each preparation made at a given flow rate are given in Table 10 below.

TABLE 10
Particle Size

| Flow | | | | | | |
|---|---|---|---|---|---|---|
| Rate* | Unimodal | Std. dev. | % std. | SDP | Std. dev. | % std. |
| (μl/min.) | mean | unimodal | Dev. | mean | SDP | dev. |
| 210 | 172 | 69.8 | 41 | 196.5 | 35.4 | 18 |
| 420 | 194.4 | 75.9 | 39 | 209 | 39.2 | 19 |
| 700 | 192.5 | 77.2 | 40 | 233.5 | 91.7 | 39 |
| 1,400 | 165.6 | 66.1 | 40 | 187.2 | 40.5 | 22 |
| 2,100 | 114.5 | 48.7 | 43 | 157 | 98.6 | 63 |
| 2,800 | 66.8 | 29.6 | 44 | 105.3 | 111.4 | 106 |
| 3,500 | 63.5 | 28.4 | 45 | 104.6 | 76 | 73 |
| 4,200 | 56.1 | 25.2 | 45 | 77.5 | 28.6 | 37 |
| 4,900 | 44.9 | 20.4 | 45 | 77.4 | 47.1 | 61 |
| 5,600 | 45 | 20.3 | 45 | 77.1 | 45.3 | 59 |
| 7,000 | 38.3 | 17.3 | 45 | 61.9 | 26.7 | 43 |
| 8,400 | 39.1 | 17.8 | 46 | 91.7 | 101.1 | 110 |

*Initial flow rate of DNA and Tween 80 stream.

The above preparations include micells which in general have a size of from about 10 nm to about 20 nm. The sizes of these micelles were counted into the determinations of mean particle sizes given above. Such micelles were are formed from the Tween 80 detergent, and could be removed by ultrafiltration from the preparations prior to the use or storage thereof.

Thus, in another experiment, a preparation of particles and micells, prepared as hereinabove described, wherein the initial flow rate of the DNA/Tween stream was 4,900 μl/min. and the initial flow rate of the polymer stream was 3,500 μl/min., and having a concentration of DNA of 20.8 μg/ml, had the following mean particle size and size distribution.

| Unimodal mean | 426 nm |
|---|---|
| Std. dev. unimodal | 19.6 |
| Std. dev. % | 46 |
| SDP mean | 75.5 |
| Std. dev. SDP | 32.6 |
| Std. dev. % | 43 |

This preparation was filtered through a 0.2μ filter, followed by concentration by ultrafiltration through an Amicon polysulfone (molecular weight 500 Kda) membrane at a flow rate of 300 μl/min. with isometric structure (Millipore Corporation, Bedford, Mass.).

After the concentration and filtration, which provided for the removal of the micells, the preparation had a DNA concentration of 450 μg/ml. The preparation was stored for 7 days, and the mean particle size and distribution was measured at the start of storage, 12 hrs., 2 days, 3 days, 7 days 16 days, and 43 days. The particle sizes are given in Table 11 below.

TABLE 11
Particle Size

| | Unimodal | Std. dev. | % std. | SDP | Std. dev. | % std. |
|---|---|---|---|---|---|---|
| Time | mean | unimodal | Dev. | mean | SDP | dev. |
| Start | 113.4 | 42.9 | 38 | 121.3 | 22.6 | 19 |
| 12 hrs. | 116.9 | 40.3 | 34 | 120 | 17.8 | 15 |
| 2 days | 111.9 | 40.4 | 36 | 122.2 | 29.5 | 24 |
| 3 days | 110.4 | 39.1 | 35 | 117.7 | 19 | 16 |
| 7 days | 112.4 | 41 | 36 | 118 | 24 | 20 |
| 16 days | 113.6 | 41.1 | 36 | 121.5 | 16.4 | 13 |
| 43 days | 110.5 | 38.3 | 35 | 117.8 | 26.5 | 22 |

The above results show that a preparation of particles produced in accordance with the procedures described in this example, remains stable over time in that the size of the particles remains essentially constant.

### EXAMPLE 43

The procedure of Example 42 was repeated, except that the DNA and Tween 80 and polyethyleneimine were flowed through a 50 μl cartridge, followed by flowing through two 150 μl cartridges contained in the mixer, and the initial flow rates of the DNA and Tween 80 stream were varied from 250 ul/min. to 3,500 μl/min. The particle sizes for each preparation made at a given flow rate are given in Table 12 below.

TABLE 12
Particle Size

| *Flow | | | | | | % |
|---|---|---|---|---|---|---|
| Rate | Unimodal | Std. Dev. | % std. | Intensity | Std. dev. | std. |
| μl/Min. | mean | unimodal | dev. | mean | intensity | dev. |
| 250 | 192.8 | 74.7 | 39 | 215 | 50.8 | 24 |
| 500 | 168.8 | 66.6 | 39 | 190 | 36.6 | 19 |
| 1,000 | 115.4 | 48.1 | 42 | 142.3 | 47.8 | 34 |
| 1,500 | 93.2 | 40.2 | 43 | 113.2 | 26.4 | 23 |
| 2,000 | 78.9 | 34.4 | 44 | 115.7 | 32.1 | 28 |
| 2,500 | 76 | 33.2 | 44 | 112.3 | 78.9 | 70 |
| 3,000 | 73 | 32.3 | 44 | 106.2 | 41.7 | 39 |
| 3,500 | 66.6 | 29.4 | 44 | 109.9 | 68.9 | 63 |

*Initial flow rate of DNA and Tween 80 stream which has a flow rate 1.4 times greater than that of the polymer stream.

From the above table, the most desired conditions were selected which provided a homogeneous preparation. These conditions were applied to produce three independent batches.

The above procedure then was repeated twice at the initial flow rate of 1,500 μl/min. for the DNA and Tween 80 stream. The results of the original experiment (Experiment 38) at a flow rate of 1,500 μl/min. and the repeated experiments (Experiments 39 and 40) are given in Table 13 below.

TABLE 13
PARTICLE SIZE

| | | | | | | % |
|---|---|---|---|---|---|---|
| Exper- | Unimodal | Std. dev | % std. | Intensity | Std. dev. | std. |
| iment | mean | unimodal | dev. | mean | intensity | dev. |
| 38 | 93.2 | 40.2 | 43 | 113.2 | 26.4 | 23 |
| 39 | 110.2 | 46.3 | 42 | 133.6 | 39.2 | 29 |
| 40 | 111.5 | 47.4 | 43 | 126.6 | 32.1 | 25 |

The above results show that the method is reproducible in that, when one mixes aqueous solutions of DNA and polymer continuously at a constant charge ratio of polymer to DNA at constant flow rates, one obtains homogenous preparations of particles of DNA and a polymer consistently, wherein each preparation includes particles having similar mean particle sizes. Thus, the method of the present invention is independent of the operator. Other methods, such as hand-mixing and pipetting, are dependent upon the skill of the operator.

The above procedure was repeated at a flow rate of 1,500 μl/min., except that such procedure was scaled up such that 20 ml of each stream was fed through the mixer. The mean particle size, as determined by the unimodal mean and the intensity mean, was as follows:

| Unimodal mean | 88.3 nm |
|---|---|
| Std. dev. | |
| unimodal | 38.2 |
| % Std. dev. | 43 |
| SDP mean | 117 nm |
| Std. dev. | |
| SDP | 37.3 |
| % std. dev. | 32 |

This preparation then was filtered through a 0.2μ filter followed by concentration by ultrafiltration through an Amicon polysulfone (molecular weight 500 Kda) membrane at a flow rate of 300 μl/min. as described in Example 42, except that, after the concentration and filtration, the preparation had a DNA concentration of 250 μg/μl. The mean particle size, as determined by the unimodal mean and the intensity mean, was as follows:

| Unimodal mean | 102.9 nm |
|---|---|
| Std. dev. | |
| unimodal | 37.6 |
| % std. dev. | 37 |
| SDP mean | 115.5 nm |
| Std. dev. | |
| SDP | 23.9 |
| % std. dev. | 21 |

The preparation again was subjected to filtration through a 0.2μ filter, followed by concentration with an Amicon polysulfone (molecular weight 500 Kda) membrane at a flow rate of 300 μl/min., after which the preparation had a DNA concentration of 870 μg/μl. The mean particle size, as determined by the unimodal mean and the intensity mean, was as follows:

| Unimodal mean | 108.6 nm |
|---|---|
| Std. dev. | |
| unimodal | 37.6 |
| % std. dev. | 35 |
| SDP mean | 117.5 nm |
| Std. dev. | |
| SDP | 25.2 |
| % std. dev. | 21 |

Thus, the above results show that ultrafiltration of the particle preparations provides a homogeneous dispersion of DNA and polymer particle. In addition, the ability to make such a preparation of homogenous particles is independent of batch size.

### EXAMPLE 44

### Preparation of Core Complexes with Linear PEI and Its Biological Activity

Linear PEI was dissolved in deionized water to obtain a final concentration of 100 mM amine as determined by an ethidium bromide displacement assay. In this assay 1 mmol is defined as the amount of PEI amine required to completely neutralize 1 mmol of DNA phosphate. From a 2.72 mg/ml stock solution of plasmid DNA (pCIluc) 221 μl was combined with 110 μl of 45.46% glucose solution and 597 μl of water. 72 μl of the PEI solution was added to the mixture and vortexed thoroughly for 20 sec, to prepare complexes that had a 4:1±ratio. Two hundred microlitres of the complex were injected into CD-1 mice via the tail-vein. Each group consisting of 5 animals received the same dose. The mice were euthanized after 5 h, their organs harvested, ground, lysed and assayed for luciferase expression as described previously.

The results are shown in FIG. 5. They show that the core complexes exhibit activity to provide gene transfer in vivo although this activity can be improved for some therapeutic applications by addition of other features of a layered colloid vector.

### EXAMPLE 45

### Preparation of Coated Core Complexes Cationic Lipid and PEG Based Fusogen Surfactants and PEG-Based Steric Surfactants and their Biological Activity

Preparation of Cationic Lipid Dispersion:

All lipids of a formulation including surfactants were dissolved in an organic solvent such as cyclohexane and mixed together at the desired ratio and then lyophilized to dryness. For example, 45 mg DOTAP and 25 mg cholesterol, or 10 mg GC-030 and 4.74 mg cholesterol were used for DOTAP:Chol and GC-030:Chol, respectively. Double distilled water was added to the lipid cake to give a final concentration of 10 mg/ml of cationic lipid (cholesterol is a neutral lipid that is not counted for calculation of lipid dispersion concentration or later for charge ratio with DNA) and allowed to hydrate at 70 C for 1 hr. The lipid dispersion was extruded through 100 nm pore carbonate membranes (Avanti Polar Lipids Inc) or vortexed for 1 min at room temperature.

Preparation of Lipoplexes:

Forty microgram of pCILuc was dissolved in 100 μl of 10% glucose and mixed by hand with different amount of lipids dispersion dissolved in 100 μl distilled H₂O. The final concentration of Glucose is 5%. The mixing was performed by added the DNA solution to the lipid solution. The charge ratio of lipids to DNA in this mixture was indicated in the text. 200 μl of DNA/lipid complex solutions was injected into mouse tail vein. Each group had 3-5 mice. Five hours later, mice were sacrificed. Spleen, liver, kidney, heart and lung were excised and placed in 2 ml centrifuge tubes (Purchased from Bio 101). After added 0.5 ml lysis buffer, organs were crushed by shaking in Fasprep FP120 (Purchased from Bio 101) for 40 sec. The homogenate was centrifuged at 14,000 rpm for 5 min in table centrifuge. The 20 μl of supernatant was used for luciferase assay. Luciferase activity was determined by using luciferase assay system kit from Promega.

Transfection in vivo:

The in vivo studies were performed by injection of 200 ul of DNA/lipid complex solutions by tail vein in either mouse or neonatal rats (3-10 days old). Each group had 5 animals. Five or 8 hours later, the blood was collected by cardiac puncture, the animals sacrificed, and other organs (e.g. lungs, liver, spleen, kidney, heart) excised surgically. Serum samples were prepared by centrifugation of coagulated blood. Organ samples were prepared by addition of 1 ml lysis buffer and homogenization with Bio 101 Fasprep FP120 for 40 sec. The homogenate was assayed directly for reporter gene activity or centrifuged at 14000 rpm in microtubes for 10 min and the supernatant used for protein activity assay.

The results are shown in FIG. 7. They show that the core complexes exhibit activity to provide gene transfer in vivo, the results obtained with DOTAP:Chol without additive, that the activity can be improved by fusogenic additives, the results obtained with added Brij, Thesit, and Tween, and the activity can be inhibited by addition of steric coating additive, the results with Chol-PEG5000. Thus some features of a layered colloid vector are illustrated.

### EXAMPLE 46

### Preparation of Coated Core Complexes with Fusogen Peptide and Their Biological Activity

Materials:

All peptides were obtained from commercial peptide synthesis company (Genemed Synthesis Inc, South San Francisco, Calif.) with at least 85% purity. Peptide K14 contains the amino acid sequence of KKK KKK KKK KKK KK (SEQ ID NO:3). Peptide K14 Fuso contains fusogenic peptide derived from influenza hemagglutinin with the amino acid sequence of GLF GAI EGF IEN GWE GWI DGW YGC KCK KKK KKK KKK KKK K (SEQ ID NO:4). Lipofectamine and lipofectin was purchased from BRL (Gaithersburg, Md.).

Method:

Transfection: BL-6 cells were seeded to each well of a 96 well plate at 10000 cells/well at one day earlier. 0.5 ug of pCIluc2 DNA and different amount of peptide (ug) or lipofecting regent (ul) as indicated was added to 50 ul of serum free medium separately. Then the peptide or lipofecting reagent-containing medium was added to the DNA containing medium. The mixture was incubated at room temperature for 30 min and then added to the cell. After 3 hr incubation, the transfection solution was removed and medium was exchanged to the serum containing one.

Luciferase activity was measured at 24 hr after the transfection with luciferase assay kit from Promega according to the recommended procedure.

The results are shown in FIG. 8. They show that the core complexes exhibit activity to provide gene transfer in vitro varies with core. The results obtained with K14 and the two commercial lipid reagents show that the cores formed by the two lipids give substantially greater expression than that formed by the K14. The results also show that the activity of the core formed by K14 can be improved by addition of a fusogenic peptide sequence to give a substantial increase in expression to parallel that by the two lipids. Thus some features of a layered colloid vector are illustrated.

### Example 47

### Preparation of Hydrazone Linkage and Acid pH Induced Cleavage (Synthesis, Cleavage Assay Methods, Results)

Preparation of 1-Acetyl-2-paramethoxyphenylhydrazone

To a stirred solution of 0.108 g of acetic hydrazide in 0.2 ml of methanol 0.33 ml of anisaldehyde was slowly added. After the addition the reaction was stirred for a further 48 h. 0.1 ml of reaction mixture was taken and added to 0.4 ml of water. 0.085 ml aliquots were then purified using C8 reverse phase hplc (Vydac 300A, 10u, 250 mm×10 mm) with solvent A as aqueous 0.025M sodium phosphate pH 7.5 and solvent B as methanol. Flow of 1 ml per minute and gradient of 55% to 95% solvent B over 35 minutes was used. The product 1-acetyl-2-paramethoxyphenylhydrazone was collected from the peak eluting at 15 minutes into the gradient to give 0.020 g of a white solid.

1¹H NMR (400 MHz, DMSO-d6) showed the prescence of two isomers of product, anti- and syn-geometrical isomers of ratio 1:1.69.

Major isomer: δ 2.17 (s, CH₃C═O), 3.79 (s, CH₃O), 6.98 (d, J=8.8, Ar), 7.59 (d, J=8.6, Ar), 7.92 (s, ArCH═N), 11.105 (s, NHAc)

Minor isomer: δ 1.92 (s, CH₃C═O), 3.795 (s, CH₃O), 6.99 (d, J=8.8, Ar), 7.61 (d, J=8.4, Ar), 8.08 (s, ArCH═N), 11.22 (s, NHAc)

For the acid hydrolysis studies 0.35 mg of anti-/syn-mixtures of 1-acetyl-2-paramethoxyphenylhydrazone was dissolved in 2 ml of 0.05M sodium citrate/potassium phosphate pH 5 containing 10% methanol. The mixture was immediately adjusted to pH 5 using NaOH and the reaction was kept at 37° C. At time intervals 0.1 ml was withdrawn and 0.3 ml of 0.25 M of potassium phosphate pH 7.5 was added to raise the pH to 7.5. Injected onto C8 reverse phase hplc (Vydac 300A, 10u, 250 mm×10 mm) with solvent A as aqueous 0.025M sodium phosphate pH 7.5 and solvent B as methanol. Flow of 1 ml per minute and gradient of 55% to 95% solvent B over 35 minutes was used. Rate of hydrolysis was determined by the peak areas of the 4-methoxybenzaldehyde and 1-acetyl-2-paramethoxyphenylhydrazone peaks.

The above acid hydrolysis studies were performed in the same manner using buffers at pH 5.5 and 6.1.

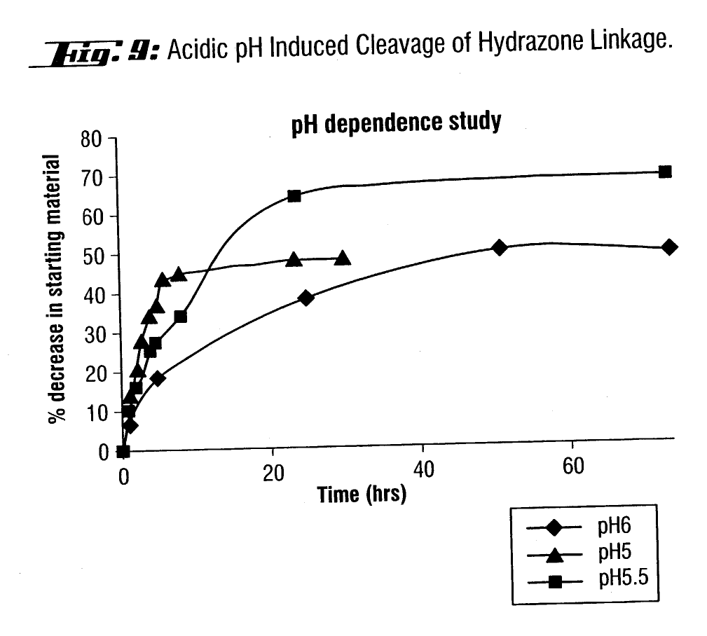

The results are shown in FIG. 9. They show that the hydrazone linkage can be hydrolyzed at acidic pH and that the rate of cleavage depends on the chemical structure of the linkage. Thus some features of a layered colloid vector where the vector changes physcial states due to exposure to acid conditions are illustrated. Some uses of the changes due to acidic conditions include loss of steric protective layers and induction of fusogenic activity.

### EXAMPLE 48

### Preparation of Core Complexes Coated with Ligand Peptide and Their Biological Activity

Synthesis of K14-RGD and K14-SST

Preparation of Complexes Including Peptide Ligand Conjugates and Ligand-Mediated Cell Binding and Uptake

Materials:

K14RGD peptide containing the amino acid sequence: KKK KKK KKK KKK KKS CRG DC (SEQ ID NO:5) with at least 90% purity was synthesized at Alpha Diagnostic International (San Antonio, Tex.). Peptide K14SMT contains the amino acid sequence: KKK KKK KKK KKK KKA d-FCY d-WKT CT (SEQ ID NO:6), and peptide K14MST contains the amino acid sequence KKK KKK KKK KKK KKA TDC RGE CF (SEQ ID NO:7). Both SMT and MST peptides were synthesized at Genemed Synthesis Inc (CA, South San Francisco) and oxidized to make circularized peptide. The peptide was purified to 90% purity by the provider. CHO (Sst+) cell line was obtained from Novartis Oncology (Dr. Friedrich Raulf). The cell line was selected to stable express human somatostatin receptor Sst2.

Method:

20000 HUVEC cells were seeded to each well of a 96 well plate and cultured for 12 hr before transfection. 0 or 2 ug of K14RGD peptide was mixed with indicated amount of Lipofectin from 0.1 ul to 4 ul in 50 ul of serum free medium for 15 min. The mixture was added to 50 ul serum free medium containing 0.5 ug pCIluc2 DNA. The poly-lipoplex was incubated for 30 min before added to the cells. The transfection solution was removed after 3 hr and serum-containing medium was added to the cells.

10000 CHO (Sst2) cells were cultured in a serum-containing medium with 0.4 mg/ml G418 for 12 hr before transfection in each well of a 96 well plate. The medium was changed to a serum free medium before transfection. Peptide was added to the cell at indicated amounts from 1 ug to 10 ug/well and incubated for 30 min before 0.5 ug pCIluc2 was added to the same medium to transfect the cells. Lipofectin at 4 ul was used as the control.

At 24 hr, luciferase activity was measured with Promega luciferase assay kit according to the recommended procedure.

Results:

The results are shown in FIGS. 25 and 26. FIG. 25 shows increased expression by addition of a peptide ligand (K14RGD) to lipofectin core complexes. FIG. 26 shows increased expression by addition of a peptide ligand (Somatostatin or SMT) to polylysine core complexes which is not observed when a mutated somatostatin sequence (MST) is used. These figures demonstrate that the core complexes may exhibit activity to one extent or another but regardless the activity of the core can be improved by addition of a targeting ligand to give a substantial increase in expression. Thus some features of a layered colloid vector are illustrated.

### EXAMPLE 49

### Preparation of NLS Moiety Coupled to Nucleic Acid

Several means can be used to couple an NLS moiety to nucleic acid some of which are illustrated in FIG. 10A and include direct conjugation to the nucleic acid and indirect through another agent that binds the nucleic acid either in a sequence specific or sequence independent means. Agents required for these means to couple an NLS to the nucleic acid include synthesis of triplex oligo-peptide, PNA-peptide, PCR fragment, plasmid DNA, restriction enzyme fragments, caping agents such as quadruplex, and spacers such as PEG and polyoxazoline.

PNA-NLS Peptide Bound to DNA:

A linear DNA fragment containing the coding region from pCIluc was prepared and amplified by PCR. The primers for the reaction were so designed that the linear fragment contained the sequences AAAGAGGG and GAGAGGAA on its 5′ and 3′ ends respectively. Peptide nucleic acid (PNA) sequences, X-O-O-TTTCTCCC-O-O-O-CCCTCTTT (SEQ ID NO:8) and Y-O-O-TTCCTCTC-O-O-O-CTCTCCTT (SEQ ID NO:9) were synthesized by solid phase synthesis at Research Genetics (Hunstville, Ala.). Here C and T are the cytosine and thymine PNA analogues and O is the 8-amino-3.6-dioxaoctanoic acid linker. X stands for the SV40 large T-antigen NLS sequence PKKKRKVEDPY (SEQ ID NO: 10), while Y is rhodamine. The two compounds were purified by HPLC and analyzed by mass spectroscopy.

The two PNA molecules were designed to form a “clamp” with the complementary 5′ and the 3′ ends of the linear DNA fragment as illustrated in FIG. 10A. The DNA-PNA complex was formed by mixing a 20 times molar excess of two PNA molecules with the linear DNA and incubating for 1 h at 37° C. The complex was then separated from the unbound material in a Centricon separator (MWCO=10,000 D,) and visualized by electrophoresis on a 1% agarose gel followed by UV irradiation to illuminate the rhodamine label. The gel was then incubated in ethidium bromide followed by UV illumination. The rhodamine and the DNA bands were seen to overlap illustrating their intimate association. The material was subsequently complexed with PEI as described earlier and used to transfect SMI and HUVEC cells in culture at various doses. The cells were lysed and luciferase expression evaluated after 24 h by methods described earlier.

The results of the transfection demonstrate clearly that the linear DNA fragment containing the PNA-NLS is far more efficient in transfecting both the cell-types tested (FIG. 10B). At the highest dose, there is not a significant difference between the expression levels attained by the PNA-NLS conjugated DNA and the control fragment, but as the dose is reduced down from 200 to 50 ng, the NLS containing DNA transfects the cells more efficiently. This construct maintains its high transfection efficiency over the whole range in both the cell-types tested, while the control fragment is down to barely above-background levels. One explanation would be that at the highest dose, the nuclear import machinery is saturated and hence there is not a significant difference between the two constructs. As the dose is decreased, the DNA containing the NLS fragment is far more actively transported into the nucleus and hence is able to maintain its high levels of transfection.

It is important to note however that this construct lacking the PNA-NLS contains a free unprotected end and may be susceptible to exonuclease degradation. For this construct, DNA degradation within the cell cannot be ruled out as a reason for the lower transfection levels observed, especially at the lower doses, when a significant fraction of the DNA may be unavailable.

Synthesis of Linear DNA—NLS Peptide Conjugate:

Strategy:

Synthesize a linear DNA fragment by PCR amplification from a plasmid DNA such that the linear DNA obtained has a conjugation site at one end and a sequence that folds into a structure that provide protection from exonucleases.

| | |
|---|---|
| 5′ XCAT GGC TCG ACA GAT CTT CAA TA 3′ (FB1)(X:C6 linker with amine) | (SEQ ID NO:11) |
| 5′ X₁X₂X₂TGG GTT TTG GGT TTT GGG TTT TGG GTT TGG ATC CGC TGT | (SEQ ID NO:12) |
| GGA ATG TG 3′ (PB)(X1:acridine, X₂, X₃:C9 linker) | |

PCR protocol: PCR amplification was carried out using standard protocol. Reaction mixture had the following reagents:

| | |
|---|---|
| 1. PCR Master Mix | 50 μl |
| 2. Sterile distilled water | 32 μl |
| 3. Primer 1 (100 ng/μl) | 8 μl |
| 4. Primer 2 (100 ng/μl) | 8 μl |
| 5. Template (1 ng/μl, 10⁶ copies) | 2 μl |

PCR Master mix contains PCR buffer 1×, 2.5U TaqPolym in Brij 35, 0.005% (v/v) dATP, dCTP, dGTP, dTTP each 0.2 mM, 10 mM Tris-HCl, 50 mM KCl, 1.5 mM MgCl₂

| PCR conditions: | | |
|---|---|---|
| 1 | 94° C. | 1 min |
| 2. | 94° C. (denaturing) | 1 min |
| 3. | 60° C. (Annealing) | 1 min |
| 4. | 72° C. (Extension) | 1 min |
| Steps 2-4 repeated 38 times | | |
| 5. | 72° C. | 2 hrs |

Conjugation of NLS Peptide to DNA through PEG2000:

The NLS peptide with amino acid sequence, PKK KRK VED PYC (SEQ ID NO: 13) was obtained from Genemed Synthesis Inc. and was synthesized using solid phase method using Fmoc chemistry. The peptide was purified to >90% purity using reverse phase HPLC. Prior to reaction with DNA, the peptide was treated with 20 mM DTT. DTT treated peptide was purified on a G25 gel filtration column in order to remove free DTT using 0.1% acetic acid as solvent. Peptide was stored in 0.1% acetic acid until its reaction with PEG conjugated DNA.

Linear PCR DNA obtained from the PCR amplification was purified by extensive dialysis against 10 mM HEPES containing 50 mM NaCl using a 50,000 MWCO dialysis tubing at 4° C. 300 μg of PCR DNA was dissolved in 2 ml 10 mM HEPES at pH 7.5, containing 1.5M NaCl. 1.5 mg of N-Hydroxy succinimide PEG vinyl sulfone (NHS-PEG2000-VS), obtained from Shearwater Polymers, dissolved in 0.1 ml DMSO (dimethyl sulfoxide) was added to DNA and stirred at 4° C. for 16 hours. The reaction mixture was transferred into a 50,000 MWCO dialysis tube and dialysed against 10 mM HPES containing 1M NaCl, with frequent change of buffer, at 4° C. in order to remove the unreacted PEG derivative.

Salt concentration in the DNA solution was raised to 2M. 1 mg of the NLS peptide dissolved in 10 mM HEPES was added to the DNA solution and the pH of the solution was adjusted to 8.0 using dilute NaOH. The reaction mixture was kept at 4° C. with sterring for 16 hours. Reaction mixture was then dialyzed extensively against 10 mM HEPES containing 2M followed by 1M NaCl. Sample was stored in 10 mM HEPES containing 1M NaCl.

### EXAMPLE 50

### Synthesis of PEI-PEG Conjugates and Effect of PEGylation on the Size and Stability of PEI/DNA Complexes

Materials and Methods

PEI (25 kD) was obtained from Aldrich Chemical Company (Milwaukee, Wis.) and Methoxy poly (ethylene glycol)-nitrophenyl carbonate (MW 5000) was obtained from Shearwater Polymers (Birmingham Ala.). Concentration of PEI solutions was determined using TNBS assay for primary amine content described below. DNA concentration was determined spectrophotometrically using a molar extinction coefficient of 13,200 mol⁻¹ cm⁻¹ per base pair at 260 nm (1OD=50 μg DNA). Particle size of the colloidal formulations were determined by light scattering measurements at 90° angle on a Coulter N4 particle sizer. Autocorrelation functions were analyzed either by unimodal analysis assuming a single population of particles or using SDP analysis assuming multiple populations using the software provided by the manufacturer.

TNBS Assay:

Reagents: TNBS: 10 mM in water, Glycine HCl or any other primary amine standard: 10 mM in H₂O, Sodium carbonate or sodium bicarbonate buffer, pH 9.0, * TNBS can be purchased as solution in Methanol (5% w/v),

Procedure:

Prepare a set of standard solutions in the concentration range 5 μM to 0.1 mM in primary amines (glycine HCl can be used for this purpose) as follows. Make 300 μl of 0.1 mM Glycine HCl in 100 mM buffer. Make several samples by a serial dilution of this sample. (eg. add 200 μl of the above sample to 100 μl buffer to make a sample at 66.66 μM concentration, transfer 200 μl of the above sample to 100 μl of buffer to obtain 44.44 μM sample and so on. Remove 200 μl from the last sample after it is made so that all the samples are at equal volume ie. 100 μl). Prepare 100 μl of the primary amine sample of unknown concentration in the same buffer in duplicate or triplicate. The concentration of this sample should be within the range of the standard curve. Add 10 μl of TNBS into each sample and vortex. Incubate at room temperature for 30 minutes and read the absorbance at 420 nm. Subtract the absorbance of the blank (ie. 10 μl TNBS diluted into 100 μl of buffer) from that of each sample. Make a standard curve with the concentration of glycine against absorbance at 420 nm. From the slope and intercept of this plot and the absorbance of the sample, the concentration of primary amines can be calculated.

Conjugation of PEI with PEG5000:

10 mg of PEI was dissolved in 100 mM NaHCO₃ at pH 9 and 61 mg of methoxy-PEG5000-nitrophenyl carbonate (sufficient to modify 5% of PEI residues) was added and reacted for 16 hours at 4° C. The reaction mixture was then dialyzed extensively against 250 mM NaCl followed by water using a dialysis bag with a 10,000 MW cut-off. Synthesis of PEI conjugate of PEG350 was carried out using a similar procedure as described for PEG5000 using nitrophenyl carbonates of PEG350, obtained from Fluka, Milwaukee, Wis. The extent of PEG conjugation was estimated using the weight of the complex and the concentration of primary amine.

Formation of Anchored DNA/PEI-PEG Complex:

Complexes of DNA/PEI-PEG containing various molar concentration of PEG were prepared by hand mixing of equal volumes of DNA and PEI/PEI-PEG mixtures, followed by vortexing for 30 to 60 seconds.

Cell Binding: Confocal Microscopy

The effect of PEG on the cellular uptake of PEI/DNA complexes was evaluated by fluorescence microscopy. A 3′-Rhodamine labeled phosphorothioate oligonucleotide (5′-AAG GAA GGA AGG-3′-Rhodamine) (SEQ ID NO:14) obtained from Oligos Etc., Wilsonville, Oreg., was used as the fluorescent marker. The labeled oligonucleotide was complexed with PEI or PEI-PEG at 4:1 (+/−) charge ratio and incubated with HUVEC cells grown on microscope cover slips in a six well plate, for three hours in serum free medium. After the three-hour incubation, cells were washed with serum free medium and were allowed to grow in the presence of growth medium for another 20 hours. These cells were then washed with PBS, fixed with 4% paraformaldehyde for 15 minutes and mounted on a hanging drop microscope slide that contain PBS in the well, with the cells facing the well and in contact with PBS. The slides were observed under a Laser Scanning Confocal 10 mg of PEI was dissolved in 100 mM NaHCO₃ at pH 9 and 61 mg of methoxy-PEG5000-nitrophenyl carbonate (sufficient to modify 5% of PEI residues) was added and reacted for 16 hours at 4° C. The reaction mixture was then dialyzed extensively against 250 mM NaCl followed by water using a 10,000 MW cut-off dialysis bag. Synthesis of PEI conjugates of PEG2000, PEG750 and PEG350 were carried out using similar procedure described for PEG5000 using nitrophenyl carbonates of the respective PEGs, obtained from Fluka. Amount of PEG conjugation was estimated comparing the weight of the complex and the concentration of primary amine.

Formation of DNA/PEI-PEG Complex:

Microscope (MRC 1000, Bio-Rad) using a 60×oil immersion objective. An Ar/Kr laser light source in combination with the optical filter settings for Rhodamine excitation and emission were used for acquisition of the fluorescence images.

Biological Activity: Transfection

Transfection efficiency of PEI and PEI-PEG complexes was studied using a plasmid DNA pCI-Luc containing Luciferase reporter gene, regulated by CMV promoter. Cells (BL6) were plated at 20000 cells/well in 96 well plates and allowed to grow to 80-90% confluency. They were then incubated with PEI or PEI-PEG/DNA complexes prepared at a charge ratio of 5 (+/−) and a DNA dose of 0.5 μg DNA per well, for 3 hours in serum free medium at 37° C. Cells were allowed to grow in the growth medium for another 20 hours before assaying for the luciferase activity. Luciferase activity in terms of relative light units was assayed using the commercially available kit (Promega) and read on a luminometer, using a 96 well format.

Results

Colloidal Stability

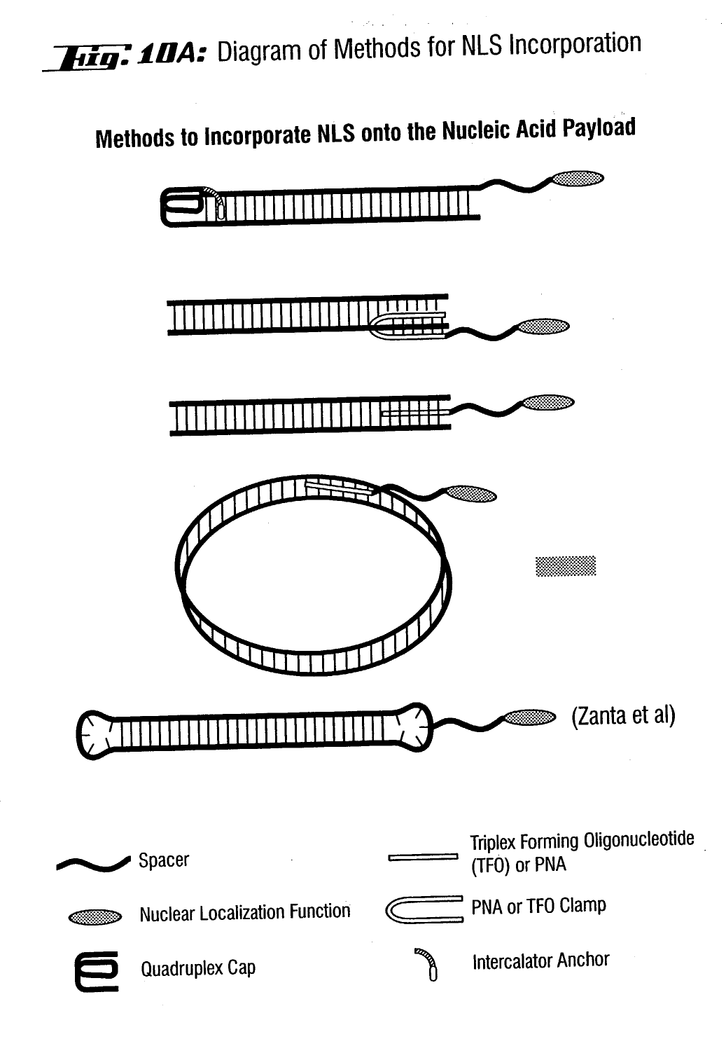

FIG. 11 shows the effect of PEG conjugation (PEGylation) on the particle size distribution of PEI/DNA complexes prepared at various charge ratios. Without PEGylation, PEI/DNA complexes have a size distribution that depends upon the charge ratio. At a net negative charge, the particles formed were quite small (about 100 nm). At near neutral charge ratios, however, PEI/DNA complexes formed or aggregated into large particles. As the charge ratio was increased to net positive, the particle size decreased, probably due to surface charge repulsion that reduces association.

With PEGylated PEI, DNA complexes are small, and the size independent of charge ratio, even at relatively high concentration of DNA, and even without using special mixing techniques. For these experiments, DNA was complexed with PEGylated PEI, where about 5% of the PEI amine residues were conjugated with PEG5000. This appears to result in PEG on the surface of these particles, effectively reducing association phenomena, even for charge neutral complexes. Without being bound by any theory, it is believed that these effects are attributable to the PEG providing a steric barrier on the surface of the complex.

It is known that PEI/DNA complexes tend to aggregate into larger particles over hours and days. This instability is an undesirable property of conventional complexes. FIG. 12 demonstrates that colloidal stability over a period of several days can be attained by PEGylation of PEI with a PEG-PEI/DNA complex prepared at 1:1 charge ratio. Mean particle size remained small even for a period of several days. These data show that PEGylation provides the long-term stability necessary for successful use of these colloidal formulations in gene therapy applications. In sum, a small amount of PEG (5 mol %) derivatization of PEI facilitates formation of small particles and provides substantial stability to the complex.

Effect of Serum

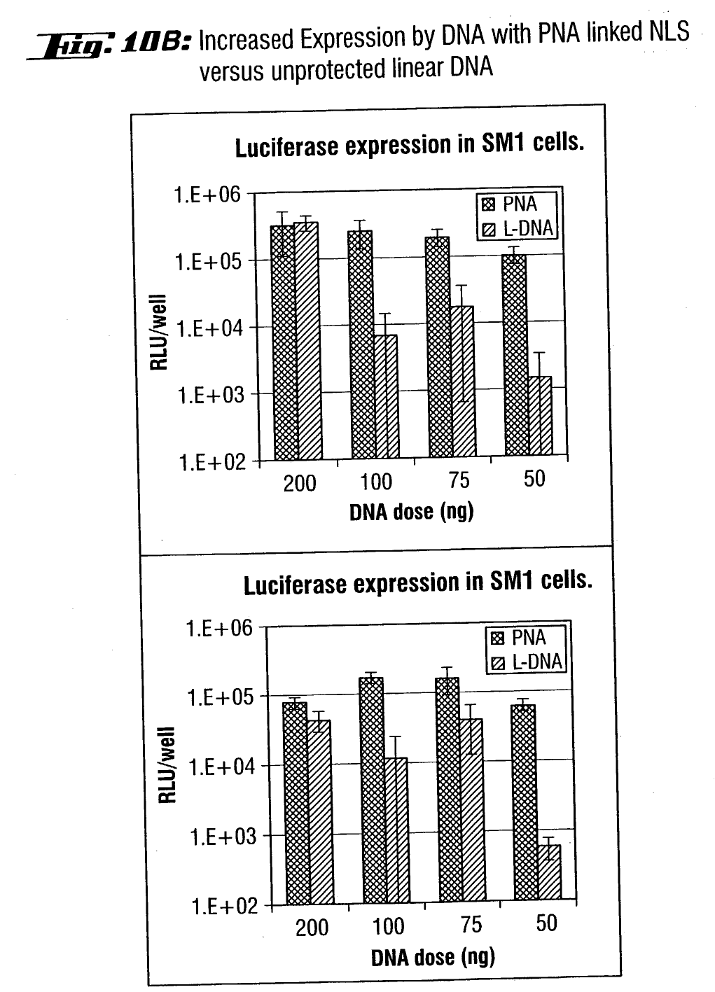

It is widely known that most positively charged DNA complexes lose their ability to transfect cells in the presence of serum. This inactivation may involve interactions with negatively charged serum leading to aggregation of these particles and/or destabilization of the complex. Anchoring of PEG to the DNA complex can be used to address this problem. FIG. 13 shows the effect of serum on the particle size distribution of PEI/DNA and PEI-PEG/DNA complexes.

On incubation with serum, conventional positively charged PEI/DNA complexes aggregate substantially, as evidenced by the increase in the average particle size distribution from about 100 nm to more than 500 nm (0 mol % PEG). This may be due to the binding of serum proteins on the surface of these complexes mediating aggregation. With the anchored complexes containing PEG5000 PEGylated PEI, protection from aggregation occured at levels greater than 1 mol %. The effect appeared to saturate by 3 mol %. This effect depends on the molecular weight of the polymer. PEG350 was ineffective to prevent the serum-mediated aggregation even up to the maximum mol % tested, as shown in the FIG. 13b. Without being bound by any theory, it is possible that the length of this polymer may be too short to provide any significant steric barrier to protein binding or the polymer may not have formed a surface coat.

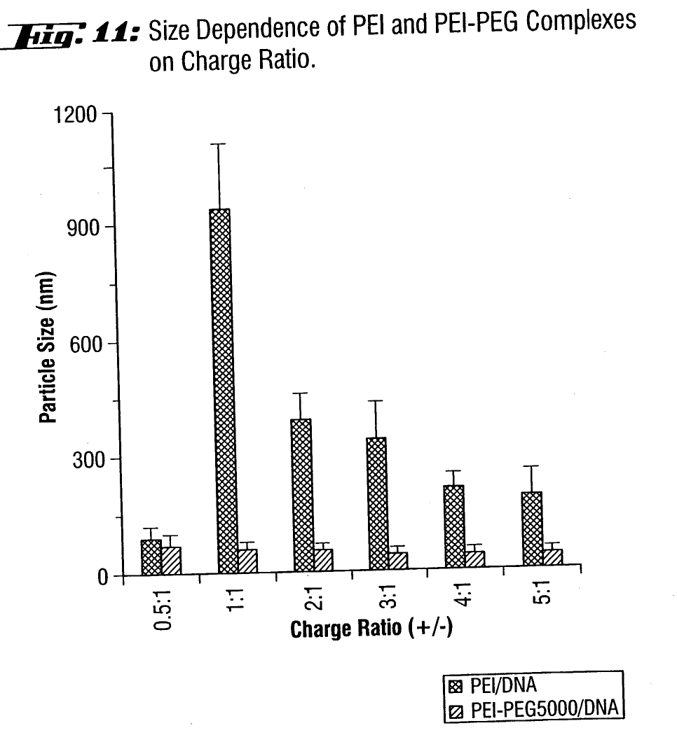

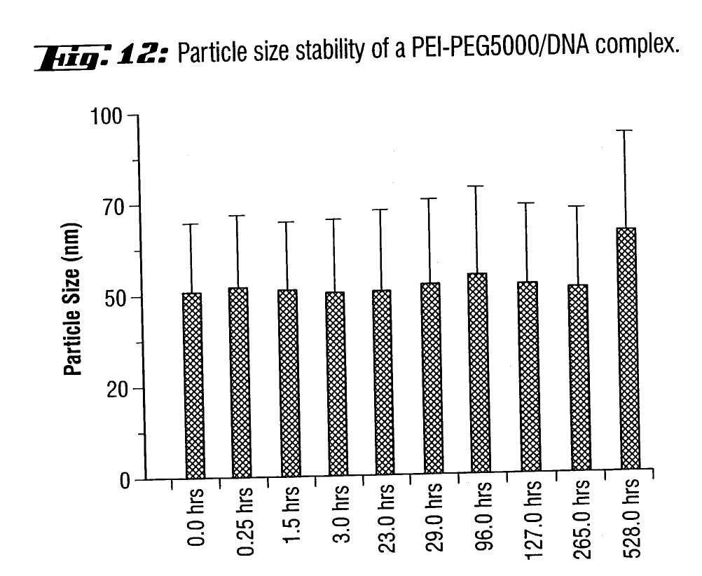

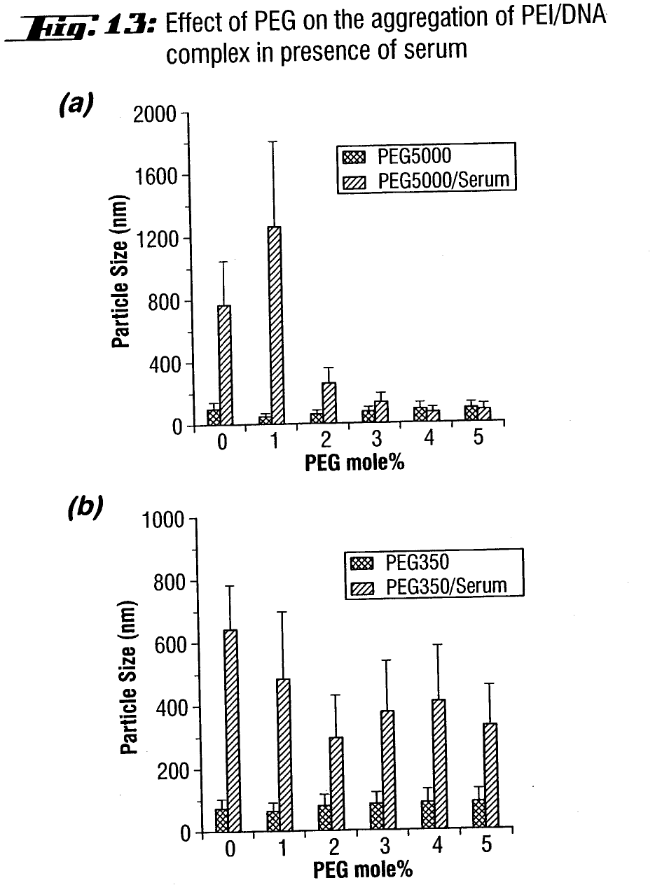

The structure of the anchored complex might be visualized as an extended polymer chain reaching above an adsorbed protein shell on the surface of the particle providing a steric barrier to particle—particle association (FIG. 14A). Thus, protein adsorption may be reduced, go unchanged, or even be increased, and the extra protein may help form a barrier to aggregation or the specific proteins increased on the surface may be beneficial.

These data demonstrate that a hydrophilic polymer, such as PEG, affects colloidal and biological property of cationic particles formed by PEI and DNA. A steric PEG coating apparently was formed on the surface of PEI/DNA complexes when the PEG was anchored to the DNA complex via a covalent bond to the PEI. This coating led to reduced particle size distribution, enhanced colloidal stability, and enhanced serum stability, all of which are desirable properties of gene delivery systems.

Biological Activity

Biological activity of PEI/DNA complexes is known to be be dependent on the charge ratio (+/−) of the complex. At net cationic charge ratios, PEI/DNA complexes, in the absence of any receptor mediated interaction, may bind to the cell surface simply through electrostatic interaction. At lower charge ratios (+/−<1), where the complex is net negatively charged and the electrostatic binding with cell surface is expected to be minimal, these complexes transfect cells very inefficiently. At high charge ratios (+/−>1), where the complex is net positively charged, electrostatic interaction with the negatively charged cell surface may be sufficient for binding and subsequent cellular uptake by endocytosis or similar mechanisms.

A PEG coating on the surface of the particles may modulate the interactions of complexes. The effect of surface PEG is to reduce electrostatic interactions and create a steric barrier. For in vitro transfections, the resulting decreased binding to the cell reduces or eliminates the uptake and inhibits expression. For in vivo systemic application, decreased protein and cell interaction should increase the blood circulation time and minimize nonspecific interactions thereby increasing the probability of the complex reaching a target tissue.

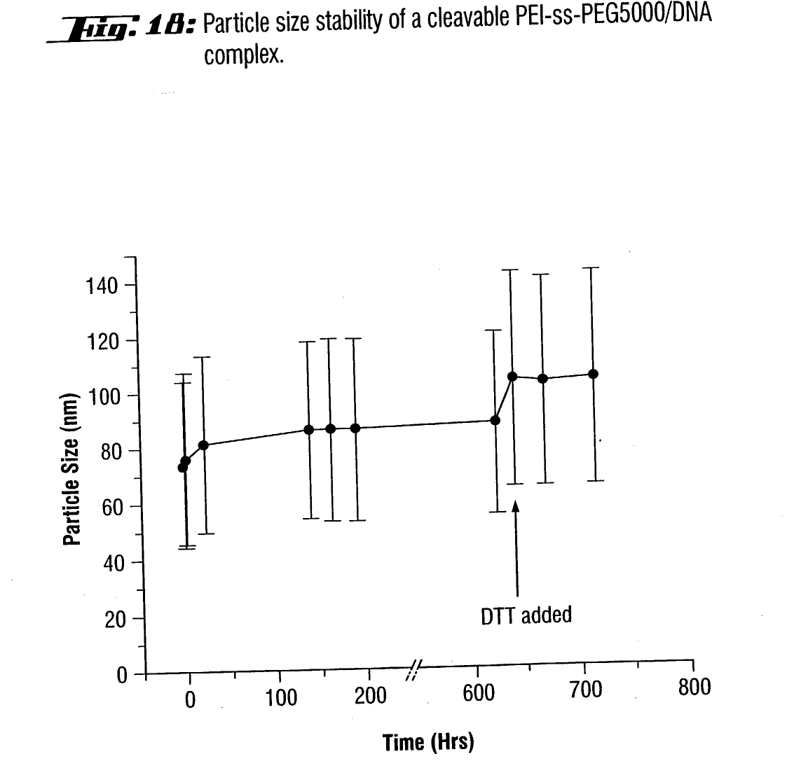

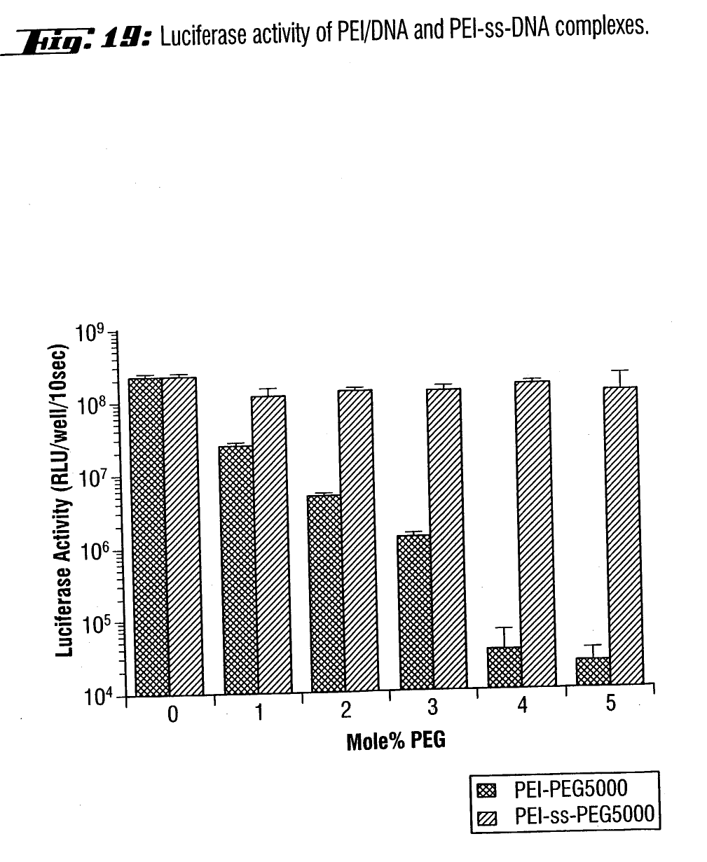

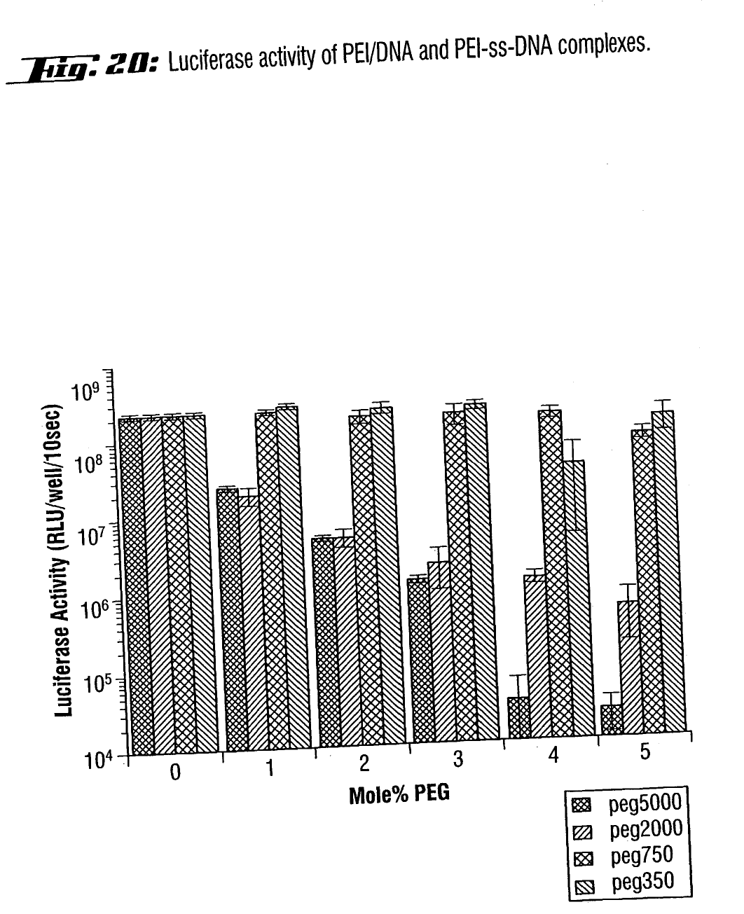

FIG. 20 shows the effect of PEGylation on the in vitro transfection efficiency of PEI/DNA complexes at a charge ratio of 5 over a range of 0 to 5 mole percent of PEGylated PEI and with different molecular weight PEG. Activity is measured as plasmid expression of the reporter gene luciferase. PEI/DNA complexes at this charge ratio transfect the cells reasonably well as shown by high luciferase expression. Presence of PEG in the complex inhibits expression in a manner highly dependent on the molecular weight and mol % of PEG. This inhibition is attributed to inhibition of binding and/or subsequent intracellular processing of the complex. A PEG molecular weight equal or greater than 2000 shows decrease in expression as the mol % of PEG in the complex is increased. The effect of 2000 molecular weight PEG seems to saturate at 3 mol % while the effect by 5000 molecular weight PEG saturates at 4 mol %. PEG350 or PEG750 up to 5% seems to have no significant effect on the activity of the complex.

Presence of a PEG coating, as described above, can influence biological activity of the complex through several ways. The polymer coat on a positively charged particle may act essentially to mask the surface charge thereby reducing binding mediated by electrostatic interaction. It can also act as a steric barrier on the surface that interferes with the binding process. A possibility also exists that steric polymers have an effect on the endosomal escape mechanism. Small molecular weight (short chain length) polymers appear to have no effect upto 5 mol %. It is likely that these small polymers provide insufficient masking. It is not known, however, whether the screening or the steric barrier, or both, is inadequate.

Accordingly, it is important to understand the mechanism by which PEG modulates the activity of the complex.

### EXAMPLE 51

### Preparation of a Sheddable PEG Coat on a PEI/DNA Complex

In addition to its stabilizing effect on DNA complexes, the presence of an anchored protective layer may impact subsequent steps in the DNA delivery process. In particular, presence of a steric layer may be detrimental to escape of the complex from the endosome, a process that may require close interaction between the complex and endosomal membrane.

One way to overcome any potential problem is to provide methods to cleave the anchored steric coat from the complex using chemical or enzymatic procedures.

This example demonstrates that a sheddable coat on a particle surface can be generated using a cleavable disulfide bond for conjugation of PEG to PEI. Example 44 showed that a steric PEG coating can be formed on the surface of PEI/DNA complexes that provides improved colloidal stability for the formulation. This example shows that the steric coat can be cleaved off, for example, under reducing conditions.

Materials and Methods

PEI (25 kD) was obtained from Aldrich Chemical Company and Methoxy poly (ethylene glycol)-nitrophenyl carbonate (MW 5000) and mercaptopolyethylene glycol 5000 monomethyl ether were obtained from Shearwater Polymers and Fluka respectively. Surface charge on the colloidal particles was determined from the electrophoretic mobility of these particles measured using a Delsa 440SX from Coulter Corporation. Other experimental conditions were as described in Example 1.

Conjugation of PEI with PEG5000:

10 mg of PEI was dissolved in 100 mM NaHCO₃ at pH 9 and 61 mg of methoxy-PEG5000-nitrophenyl carbonate was added and reacted for 16 hours at 4° C. The reaction mixture was then dialyzed extensively against 250 mM NaCl followed by water using 10,000 MWCO dialysis bag. Amount of PEG was estimated from the primary amine concentration and weight of dried sample.

PEI linked by a disulfide bond to PEG (PEI-ss-PEG) was synthesized by the following procedure. 20 mg of PEI was dissolved in 250 μl of DMSO. 8 mg of SPDP was added to this solution and allowed to react for 16 hours at 4° C., during which the reaction mixture became gel-like. 100 mg of mercaptopolyethylene glycol 5000 monomethyl ether dissolved in 2 ml of 10 mM Tris/pH 8.0 was added to the above solution and reacted for two days, during which time the gel dissolved. The sample was dialyzed extensively for 3 days against water using a 10,000 MW cut off dialysis cartridge, with frequent change of water. Percentage of conjugation was estimated using two different methods in which either: (i) the amount of PEG was estimated from the primary amine concentration and weight of dried sample; or (ii) the conjugate was treated with DTT. After removing DTT by dialysis using a 10,000 MW cut-off dialysis membrane, the ratio of primary amine to sulfhydryl ratio was determined using TNBS (RDS#) and Ellman's assay. The two procedures gave a very similar value.

Formation of DNA/PEI-PEG Complex:

Complexes of DNA/PEI-PEG containing various molar concentration of PEG were prepared by hand mixing of equal volumes of DNA and PEI/PEI-PEG mixtures followed by vortexing for 30 to 60 seconds.

Cell Binding: Confocal Microscopy

Effect of PEG on the cellular uptake of PEI/DNA complexes was evaluated by fluorescence microscopy. A 3′-Rhodamine labeled phosphorothioate oligonucleotide (5′-AAG GAA GGA AGG-3′-Rhodamine) (SEQ ID NO:14) obtained from Oligos Etc., Wilsonville, Oreg., was used as the fluorescent marker. The labeled oligonucleotide was complexed with PEI or PEI-PEG at 4:1 (+/−) charge ratio and incubated with HUVEC cells grown on microscope cover slips in a six well plate, for three hours in serum free medium. After the three-hour incubation, cells were washed with serum free medium and were allowed to grow in the presence of growth medium for another 20 hours. These cells then were washed with PBS, fixed with 4% paraformaldehyde for 15 minutes and mounted on a hanging-drop microscope slide containing PBS in the well, with the cells facing the well and in contact with PBS. The slides were observed under a Laser Scanning Confocal Microscope (MRC 1024, Bio-Rad) using a 60×oil immersion objective. An Ar/Kr laser light source in combination with the optical filter settings for Rhodamine excitation and emission was used for acquisition of the fluorescence images.

Biological Activity: Transfection

Transfection efficiency of PEI and PEI-PEG complexes was studied using a plasmid DNA pCI-Luc containing a Luciferase reporter gene, regulated by a CMV promoter. Cells (BL6) were plated at 20,000 cells/well-in 96 well plates and allowed to grow to 80-90% confluency. They then were incubated with PEI or PEI-PEG/DNA complexes prepared at a charge ratio of 5 (+/−) and a DNA dose of 0.5 μg DNA per well, for 3 hours in serum free medium at 37° C. These cells were allowed to grow in the growth medium for another 20 hours. The cells were lysed and luciferase activity was assayed (measured in relative light units) using a commercially available kit (Promega, Madison, Wis.) with a luminometer using 96 well format.

Results

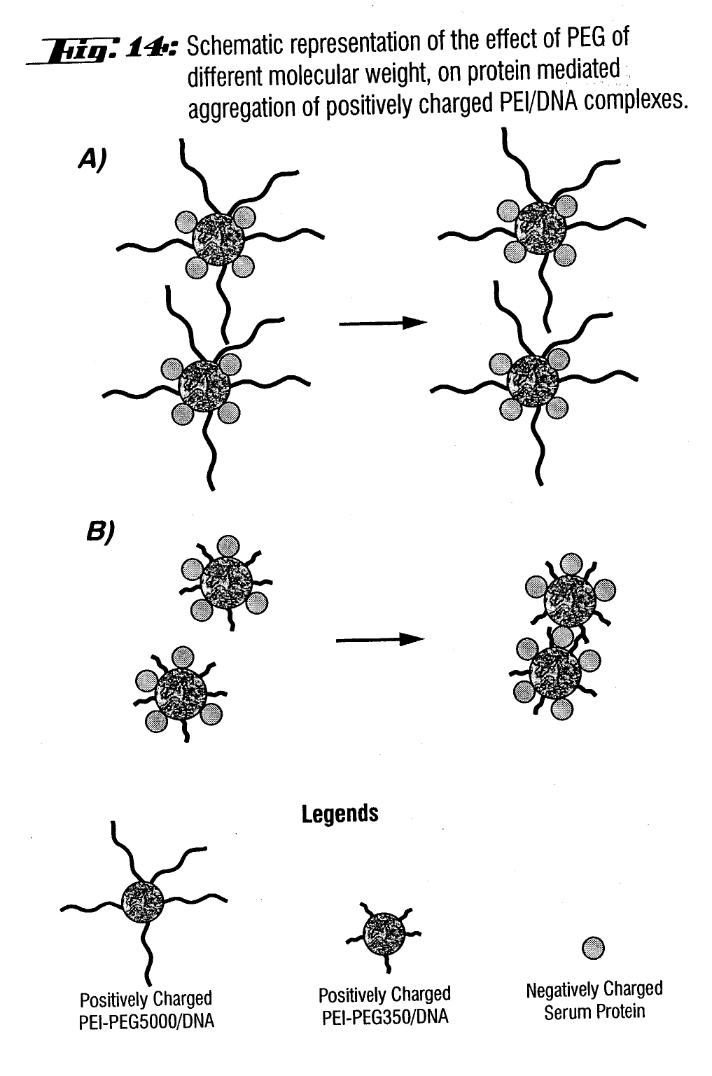

Example 44 shows that anchoring of PEG to PEI provides long term colloidal stability to a PEI/DNA complex and helps to make small particles. It also shows that the presence of a steric protective layer, such as PEG, in the complex reduces the non-specific interaction with serum proteins as well as cell surface. The results described below show the effect on the physico-chemical and biological properties of PEI/DNA complex of using a cleavable steric layer. FIG. 16 shows the particle size of a PEI/DNA complex, where the PEI contained 11% of its residues conjugated with PEG through a disulfide bond. These complexes were made at a charge ratio (+/−) of 1, where the size of conventional particles would be very large (Example 44 and FIG. 11). Contrary to a very large size, the complex was found to be relatively small, with an average size of 150 nm. When PEI-ss-PEG was pre-treated with 10 mM DTT before mixing with the DNA, particles formed were very large and precipitated out of solution within a few minutes. These data demonstrate the stabilizing effect of the anchored steric surface (PEG) and that cleavage of the PEG disulfide linker by reduction removes the surface PEG and its stabilizing effects.

For the anchored steric barrier to affect particle aggregation and reduce non-specific interaction, it must be presented at the surface of the particle. When PEGylated PEI is mixed with DNA to form particles, some of the PEG molecules could be trapped within the hydrophobic core of the complex and may not be accessible to chemical or enzymatic cleavage. However, since PEG is a hydrophilic polymer, a large fraction of it can be expected to be at the surface. Cleavage of this surface polymer may affect the particle properties significantly. One of the consequences of having the steric polymer at the surface of positively charged particles is that it masks the surface charge. Measurement of Zeta potential can be used to probe the presence of a polymer layer at the surface. Such a layer would reduce the effective surface charge, and the extent of the reduction would depend on the length of the polymer.

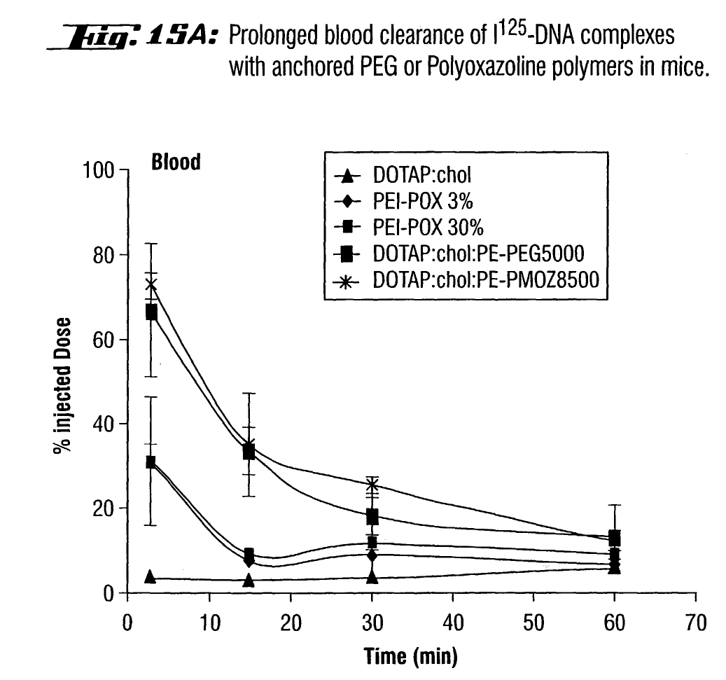

FIG. 17 shows the Zeta potential of PEI and PEI-ss-PEG5000 complexed with salmon sperm DNA at a charge ratio of 3 (+/−). A PEI/DNA at this charge ratio has a positive zeta potential of about 24 mV. DNA complexed with PEI-ss-PEG at the same charge ratio showed a much lower Zeta potential (12 mV)demonstrating the shielding of the surface charge by PEG. This complex contained 5 mol % (with respect to total amines on PEI) PEG. This zeta potential was very similar to that obtained for the PEI/DNA complex containing 5 mol % PEG, where PEG was linked to PEI through a stable linkage. Treatment of this complex with 10 mM DTT resulted in an increase in the Zeta potential (21 mV), indicating the removal of the anchored steric PEG layer from the surface. Treatment of PEI-ss-PEG with DTT before complexation with DNA gave a value similar to that of the PEI/DNA complex (22 mV). These results clearly demonstrate the presence of PEG on the surface of the complex and also its cleavability, when linked by disulfide, under reducing conditions.

Colloidal Stability

The results shown below demonstrate that presence of the cleavable anchor did not adversely affect the colloidal stability of the PEGylated complexes.

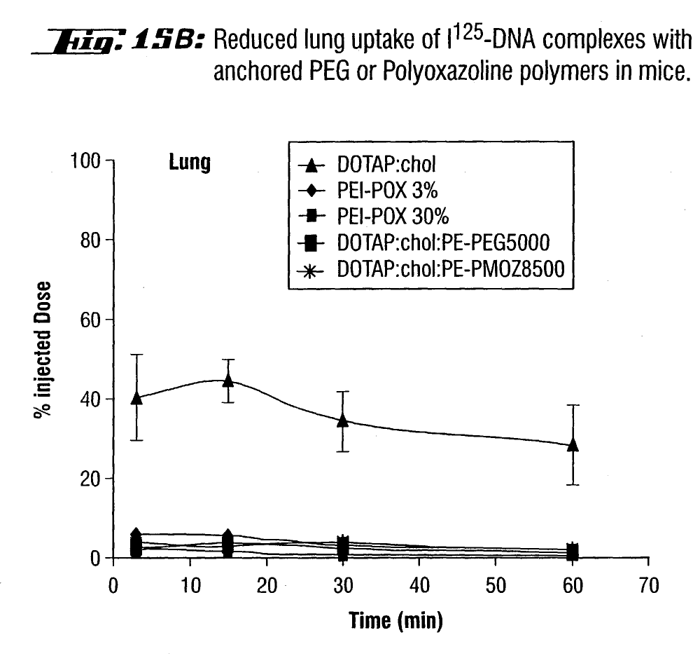

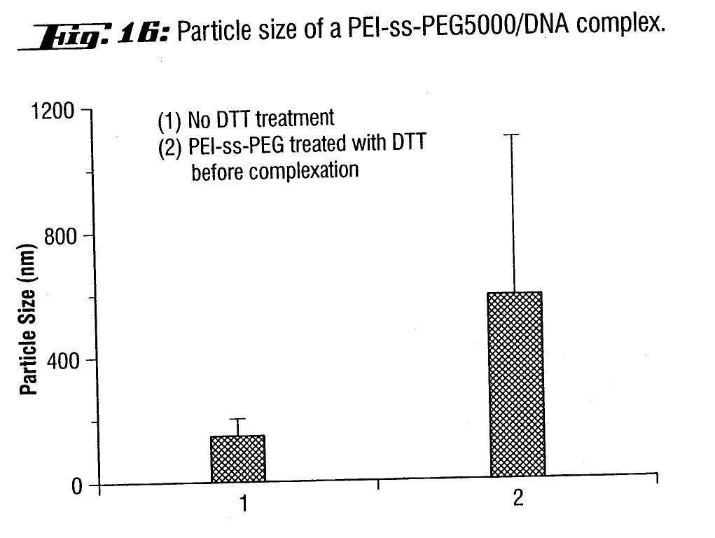

FIG. 18 shows the long term stability of PEI-ss-PEG/DNA prepared at a charge ratio of 1. Average particle size distribution of this formulation remained constant over a long period of time. This is consistent with results obtained for the PEI-PEG/DNA in Example 44. To see the effect of removing the disulfide linked PEG from the surface of the complex, 10 mM DTT was added to the sample. Average particle size increased from 88 nm to 104 nm and remained more or less unchanged with time.

Biological Activity

For PEI/DNA, in the absence of any ligands attached to the complex, initial cell binding step in DNA trafficking process is mediated by electrostatic interactions. The presence of a steric barrier (PEG) on the surface of the complex affects its physical properties in at least two distinct ways: 1) the polymer coat may physically block the interaction with cell surface and 2) it can mask surface charge so that binding mediated through electrostatic interactions is reduced. Thus a steric coat may be utilized to inhibit non-specific interactions. Use of a steric surface, for example by PEGylation of a PEI/DNA complex, can be used to inhibit unwanted biological activity. This is important since it provides a way to control non-specific interactions that lead to toxicity.

Confocal imaging using fluorescent labeling demonstrates that the likely reason for such inhibition of activity is diminished binding to cells. Binding activity may be restored by linking cell or tissue specific ligands at the distal end of the steric polymer and/or by cleaving the steric polymer off the complex surface by a chemical or enzymatic trigger. This latter method can be accomplished by conjugating PEG to PEI through a cleavable disulfide linkage.

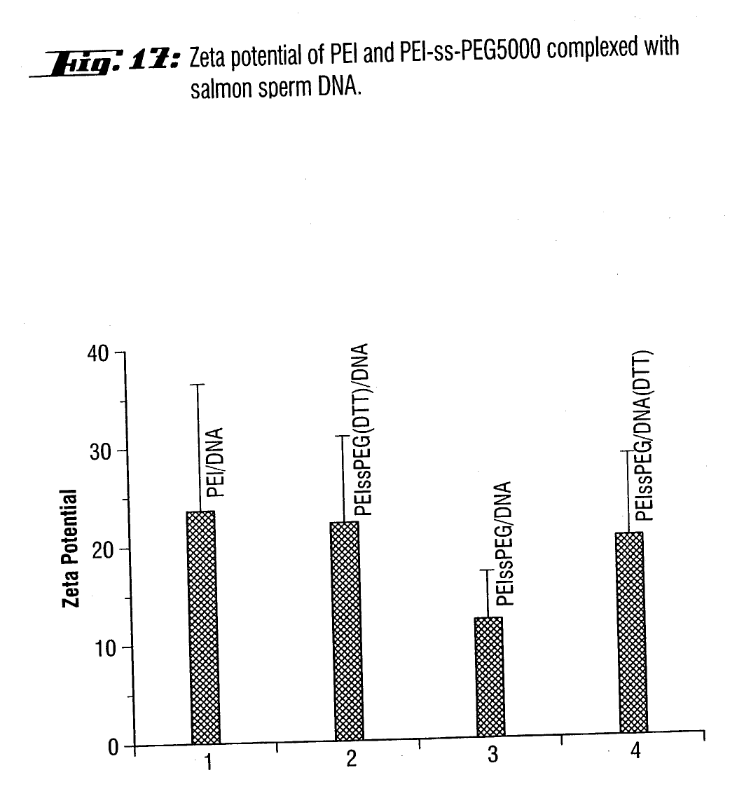

FIG. 19 shows the biological activity of PEI-ss-PEG/DNA and PEI-PEG/DNA at various mol % PEG in the complex. PEI/DNA at positive charge ratios transfected BL-6 cells efficiently. Cells transfected with PEI-PEG/DNA complex reduced the activity significantly on increasing the amount of PEG in the complex. Activity was essentially eliminated for complexes that contain >3 mol % PEG. In this case PEG was conjugated to PEI through a stable linkage. However, cells transfected with PEI-ss-PEG/DNA showed high activity even up to 5 mol % PEG. These particles retained their activity in spite of steric coating provided by conjugated PEG. Presence of PEG on the surface of the complex linked either through stable or labile linkage is expected to be inhibitory to cell binding and uptake. However, the high biological activity of PEI-ss-PEG/DNA complexes indicates that the PEG linked through disulfide bond in PEI-ss-PEG/DNA is cleaved off during the incubation or at a later stage in the DNA trafficking process.

Confocal images of HUVEC cells incubated with fluorescent labeled oligonucleotide complexed with PEI or PEI-ss-PEG showed that the PEI/oligonucleotide complex was internalized very efficiently, as indicated by the large amount of fluorescence within the cell. In contrast, cells incubated with PEI-ss-PEG/oligonucleotide complex showed considerably low internal fluorescence. Binding and uptake was greatly reduced as observed in the case of PEI-PEG/oligonucleotide complex.

### EXAMPLE 52

### Synthesis of PEI-PMOZ Conjugates and Effect of Conjugation on Surface Properties and Transfection Activity

Materials and Methods

4-Nitrophenol, bis(4-nitrophenyl) carbonate, triethylamine, dicyclohexyl carbodiimide, anhydrous acetonitrile and ahydrous dichloromethane were purchased from Aldrich (St. Louis, Mo.).

Synthesis of PMOZ and PEOZ

Poly(2-methyl-2-oxazoline) with end-group propionic acid (PMOZ-propionic acid) and poly (2-ethyl-2-oxazoline) with methyl end-group (PEOZ) were prepared as described by S. Zalipksy et al (J. Pharm. Sci. 85:133 (1996)). Gel permeation chromatography (GPC) was measured using the Hewlett Packard 1100 HPLC equipped with G-3000 PW and G-2500 PW columns (Schimadzu) placed in series and calibrated by PEG standards in water.

H-NMR spectra were measured in D₂O at 360 MHz (Spectral Data Services Inc, Champaign, Ill.).

Activation of PMOZ—Preparation of 4-nitrophenyl ester of PMOZ-propionic Acid

PMOZ-propionic acid (MW: 9100, 0.129 mmol of propionate end group) was azeotropically dried in 10 ml anhydrous acetonitrile twice. The polymer was then dissolved in 3 ml anhydrous dichloromethane and 4-nitrophenol (2.87 mmol) was added. The mixture was cooled to 0° C. and 2.62 mmol dicyclohexylcarbodiimide (DCCl) in 2 ml anhydrous dichloromethane was added. After 30 min, the mixture was allowed to warm to room temperature and allowed to incubate for 16 h. The reaction mixture was then added dropwise to 300 ml anhydrous diethyl ether while being stirred. The supernatant was discarded, the precipitate dissolved in anhydrous acetonitrile and the precipitation in diethyl ether repeated 3 times to give 4-nitrophenyl ester of PMOZ-propionic acid (0.545 g) as a white powder.

Activation of PEOZ—Preparation of 4-nitrophenyl carbonate of PEOZ

PEOZ (M.W. 8850, 0.1 mmol of hydroxyl end group) and triethylamine (0.25 mmol) were dissolved in 10 ml anhydrous acetonitrile. A solution of bis(4-nitrophenyl) carbonate (2.5 mmol) in 10 ml anhydrous acetonitrile was added with stirring while maintaining the temperature at 0° C. The mixture then was allowed to warm to room temperature and reaction continued for 20 h. The reaction mixture was then concentrated, re-dissolved in 5 ml anhydrous acetonitrile and added dropwise to an anhydrous mixture of 500 ml diethyl ether and 10 ml dichloromethane with stirring. The supernatant was removed and precipitate dissolved in 5 ml acetonitrile and re-precipitated in the ethyl acetate-dichloromethane again. The collected precipitate of the 4-nitrophenyl carbonate of PEOZ (0.59 g) was a white solid. A TLC test on silica gel plates (eluant: ethyl acetate) indicated the absence of bis(4-nitrophenyl)carbonate.

Conjugation of PMOZ with PEI

43 mg of PEI was dissolved in 0.1M bicarbonate buffer at pH 9.0. 545 mg of the activated PMOZ was added and allowed to react at room temperature overnight. Following reaction, the pH was lowered to 5 by the addition of concentrated HCl. The liberated nitrophenol was extracted by chloroform treatment 5 times. Briefly, the reaction mixture was mixed with 100 ml chloroform in a separating funnel, shaken vigorously and allowed to stand and separate into two phases. The nitrophenol was carried preferentially into the chloroform phase which was removed, followed by addition of fresh chloroform and the process was repeated. The material was then dried and re-dissolved in 10 ml deonized water followed by dialysis against 150 mM NaCl with 2 changes of buffer, followed by dialysis against deionized water with 4 changes over 2 days. The product was then lyophilized and the PMOZ loading and amine content determined by NMR.

Conjugation of PEOZ with PEI

32.035 mg of PEI was dissolved in 5 ml 0.1M borate buffer at pH 8.0. 590 mg of the activated PEOZ was dissolved in 4 ml acetonitrile and added to the PEI solution while stirring. After 5 min a precipitate was observed which disappeared upon addition of 15 ml borate buffer. The reaction mixture was allowed to react at room temperature overnight. Following reaction, the material was dried in a rotovaporator to remove all the acetonitrile. The pH was then lowered to 5 by the addition of concentrated acetic acid. The liberated nitrophenol was extracted by chloroform as described above. This was followed by further extraction with ethyl acetate to remove most of the remaining nitrophenol. The material was then dried, re-dissolved in 10 ml deionized water, and dialyzed against 0.1M acetic acid with 2 changes and then against deionized water with 4 changes over 2 days. The product was then lyophilized and the PEOZ loading and amine content determined by NMR.

Formulation of anchored DNA/PEI-PMOZ Complexes

Complexes were formed as described previously.

Biological Activity: Transfection

Biological activity was measured in BL-6 cells as described in Example 45.

Results

Surface Properties and Colloidal Stability

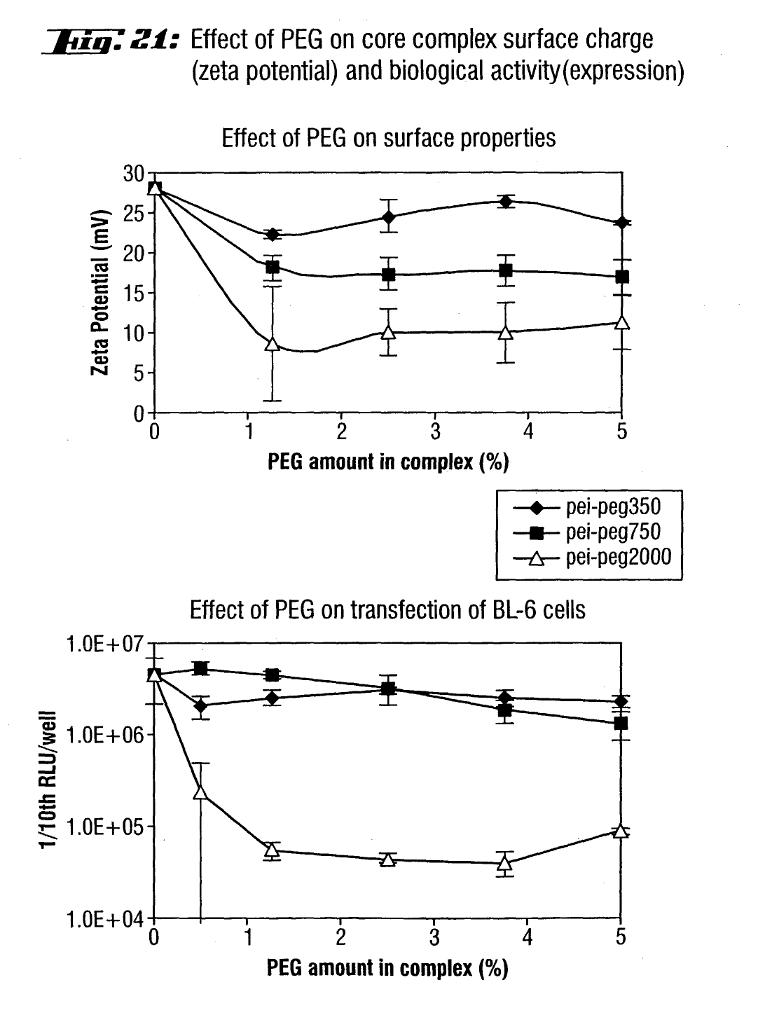

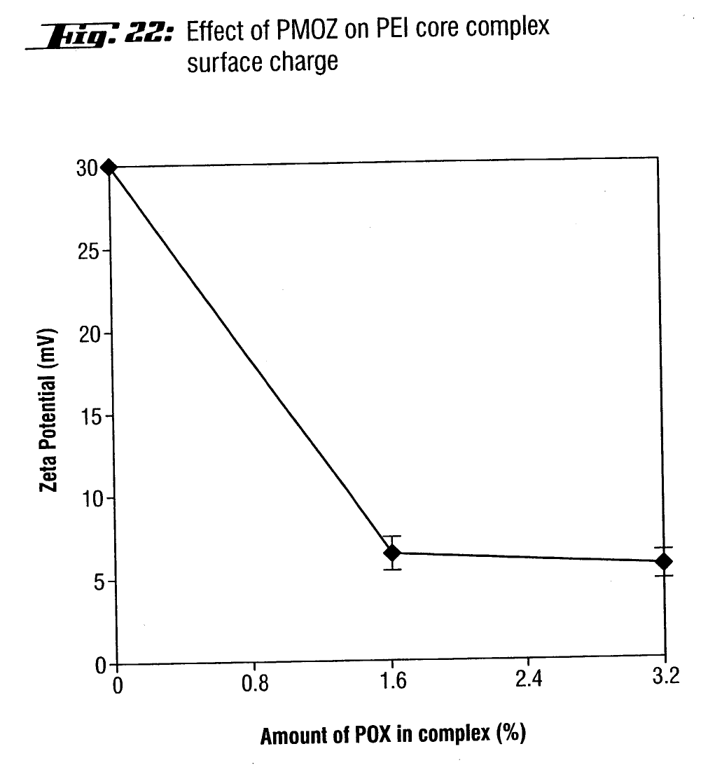

FIG. 22 shows the effect of the PMOZ on the surface properties of the complex. The complexes were formulated at a charge-ratio of 4:1 and the zeta-potential measured in 10 mM saline. With no PMOZ present, the particles demonstrate a highly positively charge surface as demonstrated by a zeta potential of +30 mV. With just a 1.6% loading of PMOZ in the complex, the zeta potential reduces to 6.46 mV. Increasing the loading to 3.2% results in a further reduction to 5.35 mV. These data suggest that, during the self-assembly process, the hydrophilic PMOZ molecules prefer to be present on the surface of the complex rather than the hydrophobic interior and thereby act a steric barrier to reduce the apparent charge presented by the surface. This hydrophilic and uncharged surface can be envisaged to reduce interactions with large serum components such as proteins. Such a phenomenon was indeed observed, as shown in FIG. 23, where 4:1 charge ratio complexes were prepared with varying amounts of PMOZ from 0 to 3.2% (in steps of 0.8) were investigated for particle-size, before and after a 2 h incubation in PBS containing 10% FBS at 37° C. The stability of the complexes in serum (as measured by the ability to maintain their size) was in direct proportion to the amount of PMOZ present in the complex. This indicates that the complexes are stable in serum, which is a critical component of targeting to specific tissues.

Blocking Non-Specific Transfection

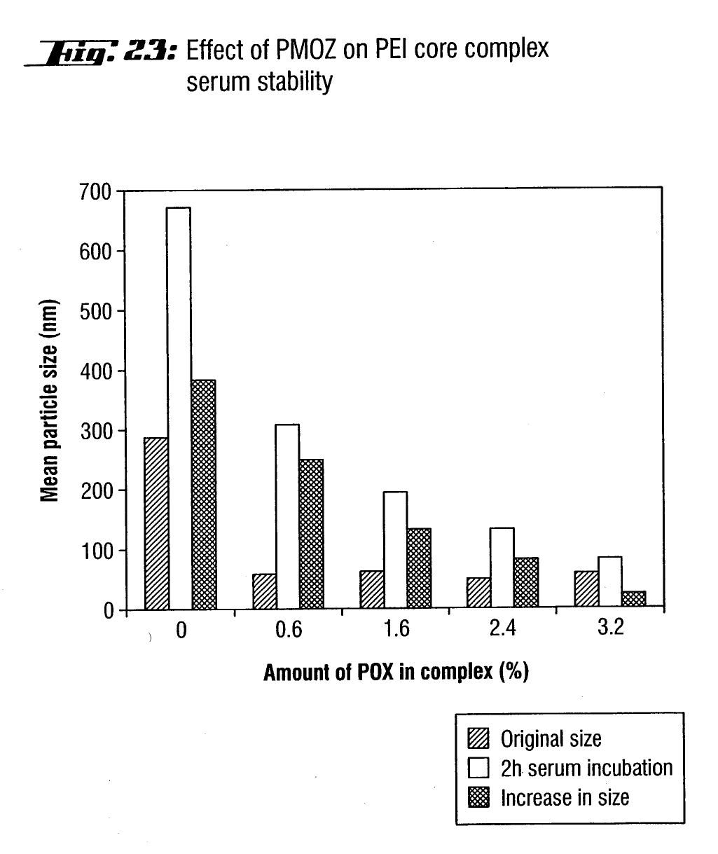

FIG. 24 shows the result obtained using the complexes described above to transfect BL-6 cells in culture. There is a clear relationship between the amount of PMOZ present in the complex and its ability to transfect cells. Increasing amounts of surface PMOZ reduced the expression levels of luciferase in these cells. As discussed above, the presence of PMOZ hinders non-specific interaction of the complexes with the cell-surface by acting as a steric and electrostatic barrier. This reduced interaction lowers uptake of the nucleic acid into the cell resulting in lower transfection levels. This allows one to design a complex that is selective to any target by the attachment of a ligand- to the distal end of the PMOZ. In this design an optimal number of ligand molecules can be appended to a steric polymer far from the surface of the particle, allowing for efficient interaction with a target receptor.

### EXAMPLE 53

### Preparation of ligand-Targeted, Layered Colloid Complexes with Outer Steric Coating

Preparation of PEI-PEG-RGD:

Synthesis and Purification:

RGD peptide with sequence, ACR GDM FGC A (SEQ ID NO: 15), cyclized through the Cys sidechains and purified to >90% by reverse phase HPLC (C 18 column)was obtained from Genemed Synthesis, S. San Francisco. 16.8 mg of the RGD peptide was dissolved in 100 mM HEPES buffer at pH 8.0. To this solution, 41 mg of VS-PEG3400-NHS (Shearwater Polymers) dissolved in dry DMSO (100 μl) was added slowly (over 30 minutes) with stirring using a syringe pump. The reaction mixture was kept stirring at room temperature for another 7 hours. 5 mg of PEI solution after adjusting the pH to 8.0 was added to the above reaction mixture. pH of the reaction mixture was raised to 9.5 and kept for stirring at room temperature for 4 days. At the end of the reaction, the reaction mixture was lyophilized.

The sample was redissolved in 5 mM HEPES at pH 7.0 containing 150 mM NaCl and passed through a G-50 gel filtration column using an elution buffer containing 5 mM HEPES and 150 mM NaCl. Void volume fraction was dialyzed extensively against 5 mM HEPES containing 150 mM NaCl using 25,000 MWCO dialysis tubing. The sample was desalted later by dialyzing against water using 3500 MWCO bag.

Estimation of Peptide Conjugation:

Amount of peptide in the conjugate was determined by estimating the sulfhydryl concentration from Cys side chains. A small fraction of the conjugate was treated with 20 mM DTT to reduce the peptide disulfide bond. This sample was then dialyzed against 0.1M acetic acid containing 1 mM EDTA using a 25000 MWCO dialysis tube, in order to remove excess DTT. After extensive dialysis, the sulfhydryl concentration was determined using Ellmen's reagent and the amine concentration due to PEI was determined using TNBS assay for primary amines. Based on these assays, peptide conjugation to the PEI was estimated to be 10%.

DNA Binding:

Ability of PEI-PEG-RGD2C to complex with DNA was verified by gel electrophoresis experiments. Complexes formed at or above a charge ratio of 1 failed to migrate into the gel, indicating complete charge neutralization of DNA due to binding of the conjugate.

Particle Size and Zeta Potential:

In order to facilitate the uptake of DNA/polycation complexes, DNA needs to be condensed into small particles that can be endocytosed by cells. Ability of PEI-PEG-RGD2C to condense DNA into small particles was studied by particle size measurements. Table 14 below shows the particle size of DNA/PEI-PEG-RGD2C at various charge ratios. Table 14 also shows the zeta potential values of DNA/PEI-PEG-RGD2C complexes at various charge ratios. Zeta potential remains low at these charge ratios indicating the formation of a steric coat that masks the surface charge of the complex.

TABLE 14

| Charge | Particle | Std. | Zeta | Std. |
|---|---|---|---|---|
| ratio | size(nM) | deviation | potential | deviation |
| 1.0:1 | 405.6 | 186.6 | −13.3 | 3.65 |
| 1.2:1 | 579.1 | 267.5 | −4.92 | 2.27 |
| 2.0:1 | 58.1 | 24.8 | 6.89 | 6.67 |
| 4.0:1 | 34.9 | 14.8 | 8.98 | 7.81 |
| 10.0:1 | 23.3 | 10.5 | 9.72 | 10.5 |

Cell Binding and Uptake:

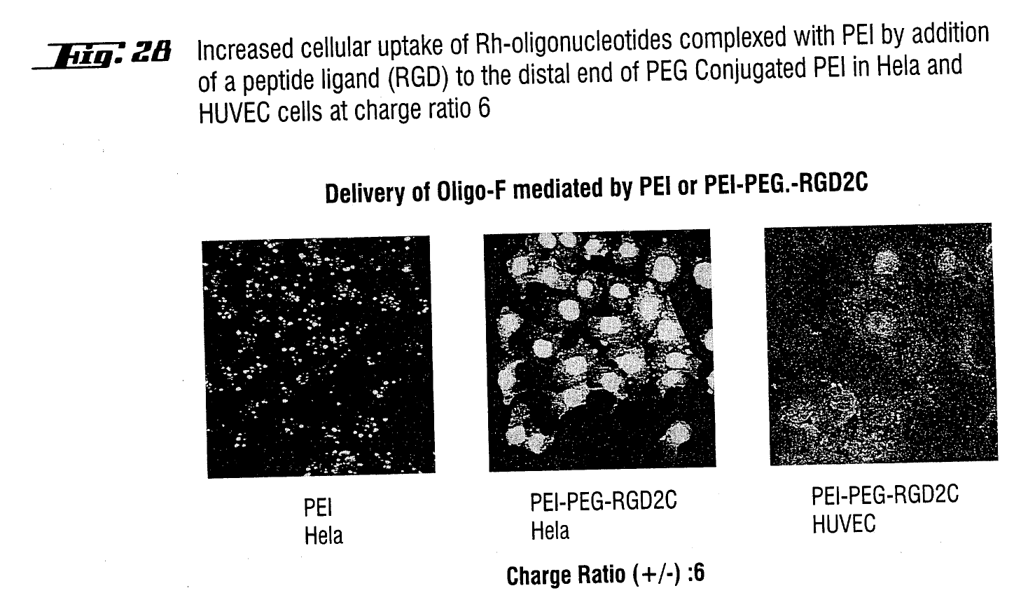

Ability of PEI-PEG-RGD2C to deliver nucleic acids to cells were studied using confocal microscopy using fluorescently labeled oligonucleotide. Confocal microscopy experiments were carried out as described earlier (Example 51). FIG. 28 Increased cellular uptake of Rh-labeled oligonucleotides complexed with PEI by addition of a peptide ligand (RGD) to the distal end of PEG-Conjugated PEI in HELA cells at charge ratio 6. The figure shows the delivery of fluorescently labeled oligonucleotide by PEI or PEI-PEG-RGD2C to Hela and HUVC cells. In Hela cells bearing integrin receptors there is a marked increase in the amount of oligonucleotide internalized when the delivery is mediated by PEI-PEG-RGD2C as compared to PEI alone. Distribution pattern is also very different. With PEI, oligonucleotide is distributed in the cytoplasm in vesicular compartments whereas with PEI-PEG-RGD2C, majority of the oligonucleotide is located in the nucleus.

### EXAMPLE 54

### Preparation of Ligand-Targeted, Layered Colloid Complexes with Sheddable Outer Steric Coating

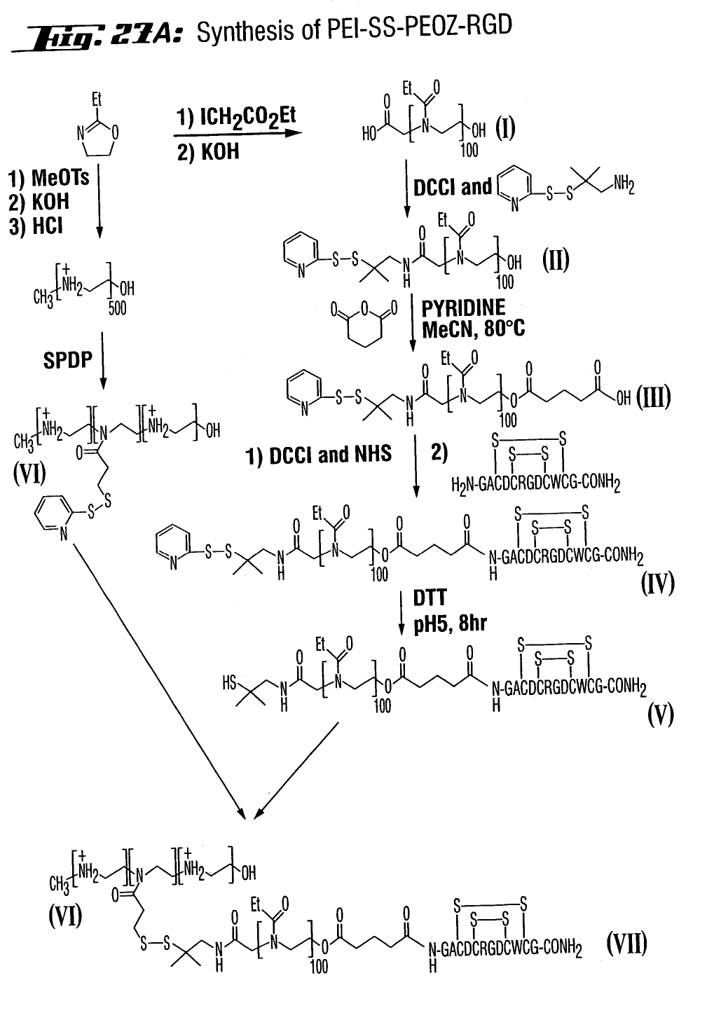

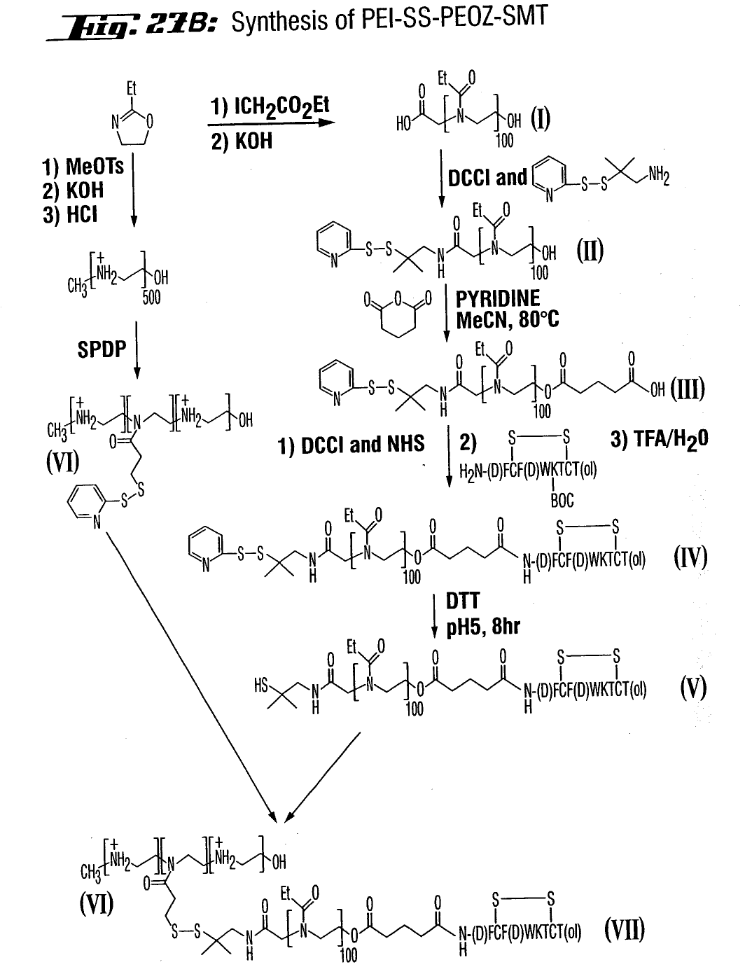

Synthesis of linear PEI conjugated with a hindered disulfide to polyethyloxazoline (PEOZ) at one end and to a peptide ligand, RGD, at the other end is illustrated in FIG. 27. As seen in FIG. 27A the preparation of PEI-SS-PEOZ-RGD involves the polymerization of 2-ethyl-2-oxazoline monomer with ethyl iodoacetate and the subsequent methanolic KOH hydrolysis to give the methylenecarboxylated PEOZ intermediate I. Condensation of the carboxylated group with 1-amino-2-methyl-2-propane[2-pyridyldithio], followed by the derivitization of the terminal hydroxyl group with glutaric anhydride and condensation of the resultant carboxylated end-group with the N-terminal amine of the RGD peptide gives the 2-pyridyl protected-SS-PEOZ-RGD intermediate IV. Reduction with 25 equivalents of dithiothreitol at pH 5 for 8 h produces the thiol HS-PEOZ-RGD V which can react with the 2-pyridyldithiopropionate derivitized linear polyethylenimine to give PEI-SS-PEOZ-RGD. It is possible to modify this last step by reducing 2-pyridyldithiopropionate derivitized linear polyethylenimine with 25 equivalents of dithiothreitol at pH 5 for 8 h and then reacting the resultant thiols on the linear polyethylenimine with the 2-pyridyl protected-SS-PEOZ-RGD intermediate IV to give the same final product PEI-SS-PEOZ-RGD.

Preparation of Methylenecarboxylated PEOZ Intermediate (I, FIG. 27A):

Polymerization reaction was conducted in a screw-cap tube that was dried under vacuo while heated prior to use. The tube was charged with 4 ml of 2-ethyl-2-oxazoline that was freshly distilled over KOH and 4 ml of dry acetonitrile. 0.85 g of freshly distilled ethyl iodoacetate was dissolved in 8 ml of dry acetonitrile and 0.80 ml of this solution was transferred to the tube containing the monomer. After this transfer the tube was purged with argon, sealed and left stirring in oil bath at 80° C. for 45 h. After cooling to room temperature 2 ml of a methanolic solution of KOH (0.5M) was added to the polymerization mixture followed by stirring at 25° C. for 4 h. 0.15 ml of glacial acetic acid was added and the mixture concentrated to a solid, redissolved in 50 ml of water and placed in 3500 molecular weight cutoff Spectral/Por dialysis membranes (Spectrum, Los Angeles, Calif.). Dialysis was against 100 mM NaCl (1×3.5L) and water (3×3.5L). The content of the dialysis bags were lyophilized and further dried under vacuo to give 3.84 g of a white solid (98%).

1H NMR (360 MHz D20) d 0.87-0.94 (m, CH3CH2C═O), 2.13-2.27 (m, CH3CH2C═O), 3.37-3.46 (m, CH2N) The sample gave a positive ion MALDI-TOF mass spectrum showing a weak, broad distribution of possible pseudo-molecular ions between approximately m/z 8,000 and 13,000 and centered at approximately m/z 10,331 (expected m/z 10,075).

Preparation of 1-Amido-2-methyl-2-propane[2-pyridyldithio]-Methylenecarboxylated-PEOZ Intermediate (II, FIG. 27A):

2 g of methylenecarboxylated PEOZ intermediate (I, FIG. 27) was dissolved in 100 ml of water and the pH adjusted with aqueous HCl to 6. The solution was concentrated under vacuo to a solid which was then dissolved in 6 ml of dry dichloromethane. 0.273 g of 1-hydroxybenzotriazole monohydrate, 0.208 g of dicyclohexylcarbodiimide and 0.253 g of 1-amino-2-methyl-2-propane[2-pyridyldithio] was added and left to stir for 48 h. The reaction mixture was filtered and the filtrate was added dropwise to 1 L of diethyl ether with stirring. After decanting, the precipitate was dissolved in 5 ml of dichloromethane and again added to 1 L of diethyl ether with stirring. After decanting, the precipitate was dissolved in 50 ml of water and placed in 3500 molecular weight cutoff Spectral/Por dialysis membranes (Spectrum, Los Angeles, Calif.). Dialysis was against 100 mM NaCl (1×3.5L) and water (2×3.5L). The content of the dialysis bags were lyophilized and further dried under vacuo to give 1.77 g of a white solid (86%).

The resulting solid was purified using C18 reverse phase hplc (Jupiter. 300A, 10u, 250 mm×10 mm) with solvent A as aqueous 0.1% trifluoroacetic acid and solvent B as acetonitrile. The flow rate was 5 ml per minute using gradient of 30% to 45% solvent B over 45 minutes. The product, 1-Amido-2-methyl-2-propane[2-pyridyldithio]-Methylenecarboxylated-PEOZ intermediate (II, FIG. 27A), was collected from the peak eluting at 20 minutes into the gradient to give 0.88 g of a white solid (43%).

¹H NMR (400 MHz D₂O) δ 0.87-0.94 (multiple triplets, J=7.2, CH₃CH₂C═O), 1.16 (bs, [CH₃]₂C), 2.13-2.28 (multiple quartets, J=7.3, CH₃CH₂C═O), 3.37-3.46 (m, CH₂N and CH₂OH), 3.92 (bs, NCH₂C═O), 7.67 (bdd, J¹/2+J²/2=6.8, 4-H pyridyl), 8.16 (bd, J=8.3, 2-H pyridyl), 8.27 (bdd, J¹=J²=8.10, 3-H pyridyl), 8.51 (bd, J=5.9, 5-H pyridyl)

Preparation of 1-Amido-2-methyl-2-propane[2-pyridyldithio]-Methylenecarboxylated-PEOZ-O-Glutaric monoester monoacid Intermediate (III, FIG. 27A)

0.05 g of 1-Amido-2-methyl-2-propane[2-pyridyldithio]-methylenecarboxylated-PEOZ intermediate (II, FIG. 27A) was dissolved in 1 ml of dry acetonitrile and 2 ml of dry toluene. The solution was concentrated in vacuo to a solid. A solution of 0.014 g of glutaric anhydride in 0.5 ml of dry acetonitrile was added followed by 0.025 ml of dry pyridine. The stirred mixture was placed in an oil bath at 80° C. for 24 h. After cooling the mixture was concentrated under vacuo to a solid, redissolved in 3 ml of aqueous 0.2 M sodium acetate pH 6.5 and applied to Sephadex™ G-25 fine (column diameter 1.6 cm and 65 cm height). Product was eluted from the gel column using water and was collected in the first fraction to give 0.04 g of a white solid (80%).

¹H NMR (400 MHz CD₃OD) δ 1.07-1.12 (multiple triplets, J=7.3, CH₃CH₂C═O), 1.31 (bs, [CH₃]₂C), 1.85-1.89 (m, OC═OCH₂CH₂CH₂CO₂H), 2.18-2.25 (m, OC═OCH₂CH₂CH₂CO₂H), 2.36-2.47 (multiple quartets, J=7.3, CH₃CH₂C═O), 3.5-3.57 (m, CH₂N and CH₂OH), 4.09 (bs, NCH₂C═O), 4.23-4.26 (m, CH₂OC═O), 7.21-7.23 (m, 4-H pyridyl), 7.76-7.81 (m, 2-H and 3-H pyridyl), 8.42 (m, 5-H pyridyl)

Preparation of 1-Amido-2-methyl-2-propane[2-pyridyldithio]-Methylenecarboxylated-PEOZ-O-Glutaric Monoester Peptidyl RGD Intermediate (IV, FIG. 27A)

0.03 g of 1-Amido-2-methyl-2-propane[2-pyridyldithio]-methylenecarboxylated-PEOZ-O-Glutaric monoester monoacid intermediate (III, FIG. 27A) is dissolved in 0.25 ml of dry chloroform and treated with 0.002 g of N-hydroxysuccinimde and 0.003 g of dicyclohexylcarbodiimide. The solution is stirred for 48 h at 25° C. and then filtered. The collected filtrate is added dropwise to stirred 100 ml of dry diethyl ether. After decanting, the precipitate is dissolved in 0.5 ml of dry acetonitrile and added to 0.008 g of the bis-cyclized GACDCRGDCWCG (SEQ ID NO: 16) carboxyl terminated amide peptide (Genmed Synthesis, South San Francisco). 0.003 g of 1-methylimidazole is added and the reaction is allowed to stir at 25° C. for 48 h. 3 ml of aqueous 0.2 M sodium acetate pH 6.5 is added and is placed in 3500 molecular weight cutoff Spectral/Por dialysis membranes (Spectrum, Los Angeles, Calif.). Dialysis is against 100 mM NaCl (2×3.5L) and water (3×3.5L). The content of the dialysis bags are lyophilized and further dried under vacuo to give 1-Amido-2-methyl-2-propane[2-pyridyldithio] methylenecarboxylated-PEOZ-O-Glutaric monoester peptidyl RGD intermediate (IV, FIG. 27A).

Preparation of 1-Amido-2-methyl-2-propanethiol methylenecarboxylated-PEOZ-O-Glutaric Monoester Peptidyl RGD Intermediate (V, FIG. 27A)

0.02 g of 1-Amido-2-methyl-2-propane[2-pyridyldithio] methylenecarboxylated-PEOZ-O-Glutaric monoester peptidyl RGD intermediate (IV, FIG. 27A) is dissolved in 0.5 ml of aqueous 0.2 M sodium acetate pH 5 containing 5 mM EDTA. The solution is purged with nitrogen and 0.008 g of dithiothreitol is added. Left to stir for 8 h and is then applied to Sephadex™ G-25 fine (column diameter 1.6 cm and 65 cm height). Product is eluted from the gel column using aqueous 0.10 M acetic acid and is collected in the first fraction to give 1-Amido-2-methyl-2-propanethiolmethylenecarboxylated-PEOZ-O-glutaric monoester peptidyl RGD intermediate (V, FIG. 27A).

Preparation of 2-pyridyldithiopropionate Derivitized Linear Polyethylenimine (VI, FIG. 27A)

A solution of 0.013 g of N-succinimidyl-3-(2-pyridyldithio)propionate (SPDP) from Pierce, Rockford Ill., in 0.5 ml of dry methanol is added to a solution of 0.022 g of free base linear polyethylenimine of MW 22 kDa in 0.25 ml of dry methanol. The reaction is stirred in the dark for 16 h. 10 ml of aqueous 0.5 M sodium acetate pH 6.5 is added and the resultant mixture is placed in 3500 molecular weight cutoff Spectral/Por dialysis membranes (Spectrum, Los Angeles, Calif.). Dialysis is against 0.5 M NaCl (2×2 L) and water (3×2 L). The content of the dialysis bags are lyophilized and further dried under vacuo to give 2-pyridyldithiopropionate derivitized linear polyethylenimine (VI, FIG. 27A).

Preparation of 1-Amido-2-methyl-2-propanedithio(polyethylenimine) methylenecarboxylated-PEOZ-O-Glutaric Monoester Peptidyl RGD Intermediate (VII, FIG. 27A)

0.01 g of 2-pyridyldithiopropionate derivitized linear polyethylenimine (VI, FIG. 27A) is dissolved in 0.1 ml of 0.2 M sodium acetate buffer pH 5 containing 0.1 M sodium chloride and 25 mM EDTA. The solution is purged with nitrogen. A solution of 0.125 g of 1-amido-2-methyl-2-propanethiol methylenecarboxylated-PEOZ O-glutaric monoester peptidyl RGD intermediate (V, FIG. 27A) in 0.5 ml of 0.2 M sodium acetate buffer pH 5 containing 0.1 M sodium chloride and 25 mM EDTA is then added. The reaction mixture is stirred for 8 h. The extent of the coupling can be determined by measuring the absorbance at 343 nm for the pyridine-2-thione that is released. Molar extinction coefficient at 343 nm=8.08×10³ M⁻³ cm⁻¹. The reaction is terminated by the addition of 0.01 g of mercaptoethanol. Further stirring is continued until all pyridine-2-thione has been released. 10 ml of aqueous 0.5 M sodium acetate pH 4 is added and the resultant mixture is placed in 25,000 molecular weight cutoff Spectral/Por dialysis membranes (Spectrum, Los Angeles, Calif.). Dialysis is against 0.5 M NaCl (2×2 L) and water (3×2 L). The content of the dialysis bags are lyophilized and further dried under vacuo to give 1-amido-2-methyl-2-propanedithio(polyethylenimine) methylenecarboxylated-PEOZ-O-Glutaric monoester peptidyl RGD intermediate (VII, FIG. 27A).

PEI-SS-PEOZ-RGD and PEI-SS-PEOZ were mixed in different ratios to obtain different molar concentrations of the ligand containing molecule. These mixtures were then combined with plasmid DNA (pCIluc) as described above to produce complexes at a 4:1+/−ratio. The complexes were diluted into a 10 mM NaCl, 1 mM EDTA solution and zeta-potential determination in the DELSA 440 (Coulter Corp. Miami, Fla.) was used to estimate the thickness of the “surface coat”. HUVEC cells were then transfected and luciferase activity assayed at 24 h, 48 h and 72 h post-transfection to determine the optimal ligand amount and differences in expression-kinetics (if any). The control for the experiment was positively-charged complexes lacking the targeting coat Ligand specificity was tested in competition-assays against free ligand and in cells that were receptor-negative. These complexes were injected via the tail vein into CD-1 mice, various organs and blood-vessels were isolated and examined for luciferase expression to see differences versus control formulations.

The invention has been disclosed broadly and illustrated in reference to representative embodiments described above. Those skilled in the art will recognize that various modifications can be made to the present invention without departing from the spirit and scope thereof.

What is claimed is:

1. A non-naturally occurring gene therapy vector comprising an inner shell comprising (1) a core complex comprising a nucleic acid and at least one complex forming reagent and wherein said vector has fusogenic activity.

2. A vector according to claim 1, further comprising a fusogenic moiety.

3. A vector according to claim 2, wherein said fusogenic moiety comprises a shell that is anchored to said core complex.

4. A vector according to claim 2, wherein said fusogenic moiety is incorporated directly in said core complex.

5. A vector according to claim 1, further comprising an outer shell moiety that stabilizes said vector and reduces nonspecific binding to proteins and cells.

6. A vector according to claim 5, wherein said outer shell moiety comprises a hydrophilic polymer.

7. A vector according to claim 5, further comprising a fusogenic moiety.

8. A vector according to claim 7, wherein said outer shell moiety is anchored to said fusogenic moiety.

9. A vector according to claim 7, wherein said outer shell moiety is anchored to said core complex.

10. A vector according to claim 5, comprising a mixture of at least two outershell reagents.

11. A vector according to claim 10, wherein each of said outershell reagents comprises a hydrophilic polymer that reduces nonspecific binding to proteins and cells, and wherein said polymers have substantially different sizes.

12. A vector according to claim 1, further compring a targeting moiety that enhances binding of said vector to a target tissue and cell population.

13. A vector according to claim 5, wherein said outer shell comprises a targeting moiety that enhances binding of said vector to a target tissue and cell population.

14. A vector according to claim 1, wherein said complex-forming reagent is selected from the group consisting of a lipid, a polymer, and a spermine analogue complex.

15. A vector according to claim 1, wherein said complex-forming reagent is a lipid selected from the group consisting of the lipids shown in FIGS. 2.1 and 2.2.

16. A vector according to claim 15, wherein said complex-forming lipid agent is selected from the group consisting of phosphatidylcholine (PC), phosphatidylethanolamine (PE), dioleoylphosphatidylethanolamine (DOPE), dioleoylphosphatidylcholine (DOPC), cholesterol and other sterols, N-1-(2,3-dioleyloxy)propyl-N,N,N-trimethylammonium chloride (DOTMA), 1,2-bis (oleoyloxy)-3-(trimethylammonia) propane (DOTAP), phosphatidic acid, phosphatidylglycerol, phosphatidylinositol, glycolipids comprising two optionally unsaturated hydrocarbon chains containing about 14-22 carbon atoms, sphingomyelin, sphingosine, ceramide, terpenes, cholesterol hemisuccinate, cholesterol sulfate, diacylglycerol, 1, 2-dioleoyl-3-dimethylammonium propanediol (DODAP), dioctadecyldimethylammonium bromide (DODAB), dioctadecyldimethylammonium chloride (DODAC), dioctadecylamidoglycylspermine (DOGS), 1,3-dioleoyloxy-2-(6-carboxyspermyl)propylamide (DOSPER), 2,3-dioleyloxy-N-[2-(sperminecarboxamido)ethyl]-N,N-dimethyl-1-propanaminium trifluoroacetate (DOSPA or Lipofectamine 7), hexadecyltrimethyl-ammonium bromide (CTAB), dimethyl-dioctadecylammonium bromide (DDAB), 1,2-dimyristyloxypropyl-3-dimethyl-hydroxy ethyl ammonium bromide (DMRIE), dipalmitoylphosphatidylethanolamylspermine (DPPES), dioctylamineglycine-spermine (C8Gly-Sper), dihexadecylamine-spermine (C18-2-Sper), aminocholesterol-spermine (Sper-Chol), 1-[2-(9(Z)-octadecenoyloxy)ethyl]-2-(8(Z)-heptadecenyl)-3-(2-hydroxyethyl)imidazolinium chloride (DOTIM), dimyristoyl-3-trimethylammonium-propane (DMTAP), 1.2-dimyristoyl-sn-glycero-3-ethylphosphatidylcholine (EDMPC or DMEPC), lysylphosphatidylethanolamine (Lys-PE), cholestryl-4-aminoproprionate (AE-Chol), spermadine cholestryl carbamate (Genzyme-67), 2-(dipalmitoyl-1,2-propandiol)-4-methylimidazole (DPIm), 2-(dioleoyl-1,2-propandiol)-4-methylimidazole (DOIm), 2-(cholestryl-1-propylamine carbamate)imidazole (ChIm), N-(4-pyridyl)-dipalmitoyl-1,2-propandiol-3-amine (DPAPy), 3β-[N—(N′,N′-dimethylaminoethane)carbamoyl]cholesterol (DC-Chol), 3β-[N—(N′,N′,N′-trimethylaminoethane)carbamoyl] cholesterol (TC-CHOL-gamma-d3), 1,2-dioleoyl-sn-glycero-3-succinate, 1,2-dioleoyl-sn-glycero-3-succinyl-2-hydroxethyl disulfide ornithine conjugate (DOGSDSO), 1,2-dioleoyl-sn-glycero-3-succinyl-2-hydroxethyl hexyl orithine conjugate (DOGSHDO), N,N^{I},N^{II},N^{III}-tetramethyl-N,N^{I},N^{II},N^{III}-tetrapalmityolspermine (TM-TPS), 3-tetradecylamino-N-tert-butyl-N′-tetradecylpropionamidine (vectamidine or diC14-amidine), N-[3-[2-(1,3-dioleoyloxy)propoxy-carbonyl]propyl]-N,N,N-trimethylammonium iodide (YKS-220), and O,O′-Ditetradecanoyl-N-(alpha-trimethylammonioacetyl)diethan olamine chloride (DC-6-14).

17. A vector according to claim 14, wherein said complex forming reagent is a compound of formula I 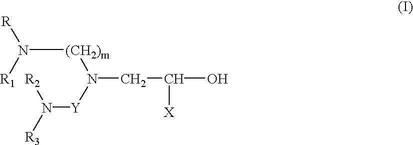
wherein m is 3 or 4;
Y signifies a group —(CH₂)ₙ—, in which n is 3 or 4, or may also signify a group —(CH₂)ₙ—, in which n is an integer from 5 to 16, or may also signify a group —CH₂—CH═CH—CH₂—, if R₂ is a group —(CH₂)₃—NR₄R₅ and m is 3;
R₂ is hydrogen or lower alkyl or may also signify a group —(CH₂)₃—NR₄R₅ if m is 3;
R₃ is hydrogen or alkyl or may also signify a group —CH₂—CH(—X′)—OH, if R₂ is a group —(CH₂)₃—NR₄R₅ and m is 3;
X and X′, independently of one another, signify hydrogen or alkyl;
the radicals R, R₁, R₄ and R₅, independently of one another, are hydrogen or lower alkyl; with the proviso that the radicals R, R₁, R₂, R₃ and X cannot all together signify hydrogen or methyl, if m is 3 and Y signifies a group —(CH₂)₃—; and their pharmaceutically acceptable salts.

18. A vector according to claim 14, wherein said complex forming reagent comprises a mixture of at least two complex forming reagents.

19. A vector according to claim 1, wherein said complex forming reagent possesses one or more additional activities selected from the group consisting of cell binding, biological membrane fusion, endosome disruption, and nuclear targeting.

20. A vector according to claim 1, wherein said nucleic acid is selected from the group consisting of a recombinant plasmid, a replication-deficient plasmid, a mini-plasmid, a recombinant viral genome, a linear nucleic acid fragment, an antisense agent, a linear polynucleotide, a circular polynucleotide, a ribozyme, a cellular promoter, and a viral genome.

21. A vector according to claim 1, wherein the core complex further comprises a nuclear targeting moiety that enhances nuclear binding and/or uptake.

22. A vector according to claim 21, wherein said nuclear targeting moiety is selected from the group consisting of a nuclear localization signal peptide, a nuclear membrane transport peptide, and a steroid receptor binding moiety.

23. A vector according to claim 21, wherein said nuclear targeting moiety is anchored to the nucleic acid in said core complex.

24. A vector according to claim 2, wherein said fusogenic moiety comprises at least one moiety selected from the group consisting of a viral peptide, an amphiphilic peptide, a fusogenic polymer, a fusogenic polymer-lipid conjugate, a biodegradable fusogenic polymer, and a biodegradable fusogenic polymer-lipid conjugate.

25. A vector according to claim 24, wherein said fusogenic moiety is a viral peptide selected from the group consisting of MLV env peptide, HA env peptide, a viral envelope protein ectodomain, a membrane-destabilizing peptide of a viral envelope protein membrane-proximal domain, a hydrophobic domain peptide segment of a viral fusion protein, and an amphiphilic-region containing peptide, wherein said amphiphilic-region containing peptide is selected from the group consisting of melittin, the magainins, fusion segments from H. influenza hemagglutinin (HA) protein, HIV segment I from the cytoplasmic tail of HIV1 gp41, and amphiphilic segments from viral env membrane proteins.

26. A vector according to claim 1, wherein said complex forming reagent is a polymer having the structure: 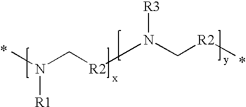
wherein R1 and R3 independently are a hydrocarbon or a hydrocarbon substituted with an amine, guanidinium, or imidazole moiety, wherein R1 and R3 can be identical or different; and
R2 is a lower alkyl group.

27. A vector according to claim 1, wherein said complex forming reagent is a polymer having the structure: 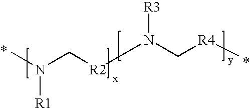
wherein R1 and R3 independently are a hydrocarbon or a hydrocarbon substituted with an amine, guanidinium, or imidazole moiety, wherein R1 and R3 can be identical or different; and
R2 and R4 independently are lower alkyl groups.

28. A vector according to claim 2, wherein said fusogenic moiety is a polymer having the structure: 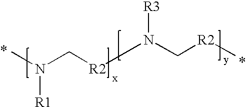
wherein R1 is a hydrocarbon or a hydrocarbon substututed with an amine, guanidinium, or imidazole moiety;
R2 is a lower alkyl group;
and R3 is a hydrocarbon or a hydrocarbon substututed with a carboxyl, hydroxyl, sulfate, or phosphate moiety.

29. A vector according to claim 2, wherein said fusogenic moiety is a polymer having the structure: 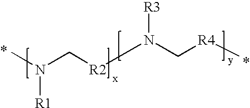
wherein R1 is a hydrocarbon or a hydrocarbon substututed with an amine, guanidinium, or imidazole moiety;
R2 and R4 independently are lower alkyl groups, and R3 is a hydrocarbon or a hydrocarbon substituted with a carboxyl, hydroxyl, sulfate, or phosphate moiety.

30. A vector according to claim 2, wherein said fusogenic moiety is a membrane surfactant polymer-lipid conjugate.

31. A vector according to claim 30, wherein said membrane surfactant polymer-lipid conjugate is selected from the group consisting of Thesit™, Brij 58™, Brij 78™, Tween 80™, Tween 20™, C₁₂E₈, C₁₄E₈, C₁₆E₈ (CₙEₙ=hydrocarbon poly(ethylene glycol) ether where C represents hydrocarbon of carbon length N and E represents poly(ethylene glycol) of degree of polymerization N), Chol-PEG 900, analogues containing polyoxazoline or other hydrophilic polymers substituted for the PEG, and analogues having fluorocarbons substituted for the hydrocarbon.

32. A vector according to claim 5, wherein said inner shell is anchored to said outer shell moiety via a covalent linkage that is degradable by chemical reduction or sulfhydryl treatment.

33. A vector according to claim 32, wherein said inner shell is anchored to said outer shell moiety via a covalent linkage that is degradable at a pH of 6.5 or below.

34. A vector according to claim 33, wherein said covalent linkage is selected from the group consisting of

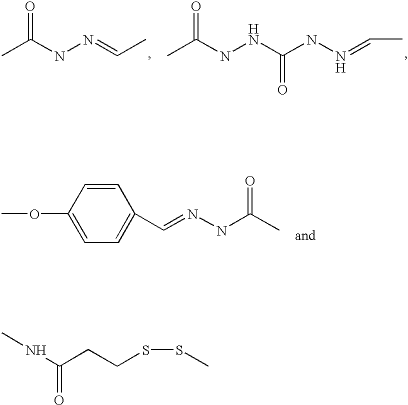

35. A vector according to claim 5, wherein said outer shell comprises a protective polymer conjugate where the polymer exhibits solubility in both polar and non-polar solvents.

36. A vector according to claim 5, wherein said outer shell comprises a protective steric polymer conjugate where the polymer is selected from the group consisting of PEG, a polyacetal polymer, a polyoxazoline, a polyoxazoline polymer block with end-group conjugation, a hydrolyzed dextran polyacetal polymer, a polyoxazoline, a polyethylene glycol, a polyvinylpyrrolidone, polylactic acid, polyglycolic acid, polymethacrylamide, polyethyloxazoline, polymethyloxazoline, polydimethylacrylamide, polyvinylmethylether, polyhydroxypropyl methacrylate, polyhydroxypropylmethacrylamide, polyhydroxyethyl acrylate, polyhydroxyethyloxazoline, polyhydroxypropyloxazoline and polyaspartamide, and a polyvinyl alcohol.

37. A vector according to claim 13, wherein said targeting element is a receptor ligand, an antibody or antibody fragment, a targeting peptide, a targeting carbohydrate molecule or a lectin.

38. A vector according to claim 37, wherein said targeting element is selected from the group consisting of vascular endothelial cell growth factor, FGF2, somatostatin and somatostatin analogs, transferrin, melanotropin, ApoE and ApoE peptides, von Willebrand's Factor and von Willebrand's Factor peptides; adenoviral fiber protein and adenoviral fiber protein peptides; PD1 and PD1 peptides, EGF and EGF peptides, RGD peptides, folate, pyridoxyl, and sialyl-Lewis^{x} and chemical analogues.

39. A compound having the formula I 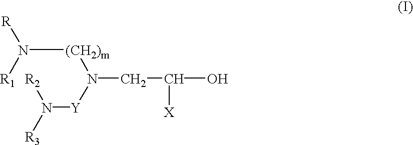
wherein m is 3 or 4; Y signifies a group —(CH₂)ₙ—, in which n is 3 or 4, or may also signify a group —(CH₂)ₙ—, in which n is an integer from 5 to 16, or may also signify a group —CH₂—CH═CH—CH₂—, if R₂ is a group —(CH₂)₃—NR₄R₅ and m is 3; R₂ is hydrogen or lower alkyl or may also signify a group —(CH₂)₃—NR₄R₅ if m is 3; R₃ is hydrogen or alkyl or may also signify a group —CH₂—CH(—X′)—OH, if R₂ is a group —(CH₂)₃—NR₄R₅ and m is 3; X and X′, independently of one another, signify hydrogen or alkyl; and the radicals R, R₁, R₄ and R₅, independently of one another, are hydrogen or lower alkyl; with the proviso that the radicals R, R₁, R₂, R₃ and X cannot all together signify hydrogen or methyl, if m is 3 and Y signifies a group —(CH₂)₃—; and their pharmaceutically acceptable salts.

40. A pharmaceutical composition comprising a vector according to claim 1, together with a pharmaceutically acceptable diluent or excipient.

41. A method for forming a self-assembling core complex according to claim 1, comprising the step of feeding a stream of a solution of a nucleic acid and a stream of a solution of a core complex-forming moiety into a static mixer, wherein the streams are split into inner and outer helical streams that intersect at several different points causing turbulence and thereby promoting mixing that results in a physicochemical assembly interaction.

42. A method of treating a disease in a patient, comprising administering to said patient a therapeutically effective amount of a vector according to claim 1.

43. A non-naturally occurring gene therapy vector comprising an inner shell comprising: (1) a core complex comprising a nucleic acid and at least one complex forming reagent; (2) a nuclear targeting moiety; (3) a fusogenic moiety; and (4) an outer shell comprising (i) a hydrophilic polymer that stabilizes said vector and reduces nonspecific binding to proteins and cells and (ii) a tageting moiety that provides binding to target tissues and cells, wherein said outer shell is linked via a cleavable linkage that enables the outer shell to be shed.
